(19) United States
(12) Patent Application Publication
(10) Pub. No.: US 2016/0151335 A1
(43) Pub. Date: Jun. 2, 2016

(54) METHODS OF MODULATING CFTR ACTIVITY

(71) Applicant: PROTEOSTASIS THERAPEUTICS, INC., Cambridge, MA (US)

(72) Inventors: Bradley Tait, Malden, MA (US); Matthew Cullen, Braintree, MA (US)

(73) Assignee: Proteostasis Therapeutics, Inc., Cambridge, MA (US)

(21) Appl. No.: 14/900,345

(22) PCT Filed: Jun. 25, 2014
(86) PCT No.: PCT/US2014/044100

§ 371 Date: Dec. 21, 2015

Related U.S. Application Data
(60) Provisional application No. 61/907,155, filed on Nov. 21, 2013.
(60) Provisional application No. 61/859,984, filed on Jul. 30, 2013.
(60) Provisional application No. 61/839,772, filed on Jun. 26, 2013.

(51) Int. Cl. A61K 31/422; A61K 31/443; A61K 31/4439; A61K 31/5377; C07D 413/12; A61K 31/42

(52) U.S. Cl. CPC A61K 31/422; C07D 413/12; A61K 31/42; A61K 31/4439; A61K 31/5377; A61K 31/443

(57) ABSTRACT

The invention encompasses methods of modulating CFTR activity in a subject in need thereof comprising administering an effective amount of a compound of Formula (I). The invention also encompasses methods of treating a condition associated with CFTR activity or condition associated with a dysfunction of proteostasis comprising administering to a subject an effective amount of a compound of Formula (I).

### RELATED APPLICATIONS

This application claims the benefit of U.S. Provisional Application No. 61/839,772 filed on Jun. 26, 2013, U.S. Provisional Application No. 61/859,894 filed on Jul. 30, 2013, and U.S. Provisional Application No. 61/907,155 filed on Nov. 21, 2013. The entire teachings of the above applications are incorporated herein by reference.

### BACKGROUND OF THE INVENTION

Cells normally maintain a balance between protein synthesis, folding, trafficking, aggregation, and degradation, referred to as protein homeostasis, utilizing sensors and networks of pathways (Sitia et al., Nature 426: 891-894, 2003; Ron et al., Nat Rev Mol Cell Biol 8: 519-529, 2007). The cellular maintenance of protein homeostasis, or proteostasis, refers to controlling the conformation, binding interactions, location and concentration of individual proteins making up the proteome. Protein folding in vivo is accomplished through interactions between the folding polypeptide chain and macromolecular cellular components, including multiple classes of chaperones and folding enzymes, which minimize aggregation (Wiseman et al., Cell 131: 809-821, 2007). Whether a given protein folds in a certain cell type depends on the distribution, concentration, and subcellular localization of chaperones, folding enzymes, metabolites and the like (Wiseman et al.). Cystic fibrosis and other maladies of protein misfolding arise as a result of an imbalance in the capacity of the protein homeostasis (proteostasis) environment to handle the reduced energetic stability of misfolded, mutated proteins that are critical for normal physiology (Balch et al., Science 319, 916-9 (2008); Powers, et al., Annu Rev Biochem 78, 959-91 (2009); Hutt et al., FEBS Lett 583, 2639-46 (2009)).

Cystic Fibrosis (CF) is caused by mutations in the cystic fibrosis transmembrane conductance regulator (CFTR) gene which encodes a multi-membrane spanning epithelial chloride channel (Riordan et al., Annu Rev Biochem 77, 701-26 (2008)). Approximately ninety percent of patients have a deletion of phenylalanine (Phe) 508 (ΔF508) on at least one allele. This mutation results in disruption of the energetics of the protein fold leading to degradation of CFTR in the endoplasmic reticulum (ER). The ΔF508 mutation is thus associated with defective folding and trafficking, as well as enhanced degradation of the mutant CFTR protein (Qu et al., J Biol Chem 272, 15739-44 (1997)). The loss of a functional CFTR channel at the plasma membrane disrupts ionic homeostasis (Cl⁻, Na⁺, HCO₃⁻) and airway surface hydration leading to reduced lung function (Riordan et al.). Reduced periciliary liquid volume and increased mucus viscosity impede mucociliary clearance resulting in chronic infection and inflammation, phenotypic hallmarks of CF disease (Boucher, J Intern Med 261, 5-16 (2007)). In addition to respiratory dysfunction, ΔF508 CFTR also impacts the normal function of additional organs (pancreas, intestine, gall bladder), suggesting that the loss-of-function impacts multiple downstream pathways that will require correction.

In addition to cystic fibrosis, mutations in the CFTR gene and/or the activity of the CFTR channel has also been implicated in other conditions, including for example, congenital bilateral absence of vas deferens (CBAVD), acute, recurrent, or chronic pancreatitis, disseminated bronchiectasis, asthma, allergic pulmonary aspergillosis, smoking-related lung diseases, such as chronic obstructive pulmonary disease (COPD), dry eye disease, Sjogren's syndrome and chronic sinusitis, (Sloane et al. (2012), PLoS ONE 7(6): e39809.doi:10.1371/journal. pone.0039809; Bombieri et al. (2011), J Cyst Fibros. 2011 June; 10 Suppl 2:S86-102; (Albert et al. (2008). Clinical Respiratory Medicine, Third Ed., Mosby Inc.; Levin et al. (2005), Invest Ophthalmol Vis Sci., 46(4):1428-34; Froussard (2007), Pancreas 35(1): 94-5).

There remains a need in the art for methods of modulating CFTR activity and for methods of treating CF, other CFTR-related diseases, and other maladies of protein misfolding.

### SUMMARY OF THE INVENTION

The present invention is based, in part, on the discovery that compounds having the Formula (I) affect cystic fibrosis transmembrane conductance regulator (CFTR) activity as measured in human bronchial epithelial (hBE) cells.

In some embodiments, the present invention is directed to a method of modulating cystic fibrosis transmembrane conductance regulator (CFTR) activity in a subject in need thereof comprising administering to said subject an effective amount of a compound having the Formula (I):

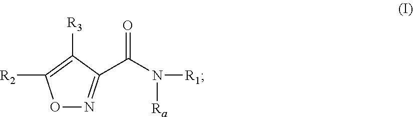

or a pharmaceutically acceptable salt, prodrug or solvate thereof, wherein:

R₁ is selected from the group consisting of:

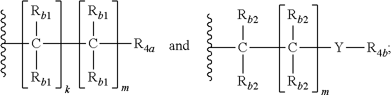

R₂ is selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted aryl, halo, OR_{c}, NR_{d}R_{d}, C(O)OR_{c}, NO₂, CN, C(O)R_{c}, C(O)C(O)R_{c}, C(O)NR_{d}R_{d}, NR_{d}C(O)R_{c}, NR_{d}S(O)ₙR_{c}, N(R_{d})(COOR_{c}), NR_{d}C(O)C(O)R_{c}, NR_{d}C(O)NR_{d}R_{d}, NR_{d}S(O)ₙNR_{d}R_{d}, NR_{d}S(O)ₙR_{c}, S(O)ₙR_{c}, S(O)ₙNR_{d}R_{d}, OC(O)OR_{c}, (C═NR_{d})R_{c}, optionally substituted heterocyclic and optionally substituted heteroaryl;

R₃ is selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted aryl, halo, OR_{c}, NR_{d}R_{d}, C(O)OR_{c}, NO₂, CN, C(O)R_{c}, C(O)C(O)R_{c}, C(O)NR_{d}R_{d}, NR_{d}C(O)R_{c}, NR_{d}S(O)ₙR_{c}, N(R_{d})(COOR_{c}), NR_{d}C(O)C(O)R_{c}, NR_{d}C(O)NR_{d}R_{d}, NR_{d}S(O)ₙNR_{d}R_{d}, NR_{d}S(O)ₙR_{c}, S(O)ₙR_{c}, S(O)ₙNR_{d}R_{d}, OC(O)OR_{c}, (C═NR_{d})R_{c}, optionally substituted heterocyclic and optionally substituted heteroaryl;

or alternatively, R₂ and R₃ can be taken together with the carbon atoms to which they are attached to form a fused, optionally substituted 3 to 12 membered cyclic group selected from the group consisting of optionally substituted C₃-C₁₂ cycloalkenyl, optionally substituted heterocyclic, optionally substituted aryl and optionally substituted heteroaryl;

R₄ₐ is selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted aryl, halo, OR_{c}, S(O)ₙR_{c}, NR_{d}R_{d}, C(O)OR_{c}, NO₂, CN, C(O)R_{c}, C(O)C(O)R_{c}, C(O)NR_{d}R_{d}, NR_{d}C(O)R_{c}, NR_{d}S(O)R_{c}, N(R_{d})(COOR_{c}), NR_{d}C(O)C(O)R_{c}, NR_{d}C(O)NR_{d}R_{d}, NR_{d}S(O)ₙR_{d}R_{d}, NR_{d}S(O)ₙR_{c}, S(O)NR_{d}R_{d}, OC(O)OR_{c}, (C═NR_{d})R_{c}, optionally substituted heterocyclic and optionally substituted heteroaryl;

R_{4b} is selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted aryl, optionally substituted heterocyclic and optionally substituted heteroaryl;

Rₐ is selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, C(O)OR_{c}, C(O)R_{c}, C(O)C(O)R_{c} and S(O)ₙR_{c};

or alternatively, Rₐ and the nitrogen atom to which it is attached is taken together with an adjacent C(R_{b1})(R_{b1}) or C(R_{b2})(R_{b2}) to form an optionally substituted, 4- to 12-membered heterocyclic ring containing one or more ring nitrogen atoms, wherein said heterocyclic ring optionally contains one or more ring heteroatoms selected from oxygen and sulfur;

Each R_{b1} and R_{b2} is independently selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, halo, OR_{c}, NR_{d}R_{d}, C(O)OR_{c}, NO₂, CN, C(O)R_{c}, C(O)C(O)R_{c}, C(O)NR_{d}R_{d}, NR_{d}C(O)R_{c}, NR_{d}S(O)ₙR_{c}, N(R_{d})(COOR_{c}), NR_{d}C(O)C(O)R_{c}, NR_{d}C(O)NR_{d}R_{d}, NR_{d}S(O)ₙNR_{d}R_{d}, NR_{d}S(O)ₙR_{c}, S(O)ₙR_{c}, S(O)ₙNR_{d}R_{d}, OC(O)OR_{c} and (C═NR_{d})R_{c}; or alternatively, two geminal R_{b1} groups or two geminal R_{b2} groups and the carbon to which they are attached are taken together to form a C(O) group, or yet alternatively, two geminal R_{b1} groups or two geminal R_{b2} groups are taken together with the carbon atom to which they are attached to form a spiro C₃-C₁₂ cycloalkyl, a spiro C₃-C₁₂ cycloalkenyl, a spiro heterocyclic, a spiro aryl or spiro heteroaryl, each optionally substituted;

Each R_{c} is independently selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl and optionally substituted heteroaryl;

Y is selected from the group consisting of S(O)ₙ, NR_{d}, NR_{d}S(O)ₙ, NR_{d}S(O)ₙNR_{d}, NR_{d}C(O), NR_{d}C(O)O, NR_{d}C(O)C(O), NR_{d}C(O)NR_{d}, S(O)ₙNR_{d}, and O;

Each R_{d} is independently selected from the group consisting of hydrogen, optionally substituted C₁-C₁₀ alkyl, optionally substituted C₂-C₁₀ alkenyl, optionally substituted C₂-C₁₀ alkynyl, optionally substituted C₁-C₁₀ alkoxy, optionally substituted C₃-C₁₂ cycloalkyl, optionally substituted C₃-C₁₂ cycloalkenyl, optionally substituted heterocyclic, optionally substituted aryl and optionally substituted heteroaryl; or two geminal R_{d} groups are taken together with the nitrogen atom to which they are attached to form an optionally substituted heterocyclic or an optionally substituted heteroaryl;

k is 0 or 1;

m is 0, 1, 2, 3, 4, or 5;

each n is independently 0, 1 or 2.

In some embodiments, the CFTR activity is enhanced. In additional embodiments, the activity of a mutant CFTR is enhanced. In some aspects, the mutant CFTR is ΔF508 CFTR.

In certain embodiments, the invention is directed to treating a subject suffering from a condition associated with CFTR activity comprising administering an effective amount of a compound of Formula (I). In additional embodiments, the invention encompasses a method of treating a subject suffering from a disease associated with decreased or deficient CFTR activity. In some embodiments, the subject is suffering from cystic fibrosis. In further embodiment, the invention is directed to a method of treating a subject suffering from a disease that can be ameliorated by suppressing CFTR activity. In some embodiments, the subject is suffering from a secretory diarrhea or polycystic kidney disease.

The present invention also encompasses an enantiomerically pure compound selected from (S)-5-phenyl-N-((tetrahydrofuran-2-yl)methyl)isoxazole-3-carboxamide (Compound 2) and (R)-5-phenyl-N-((tetrahydrofuran-2-yl)methyl)isoxazole-3-carboxamide (Compound 3). The chemical structures of these compounds are shown below:

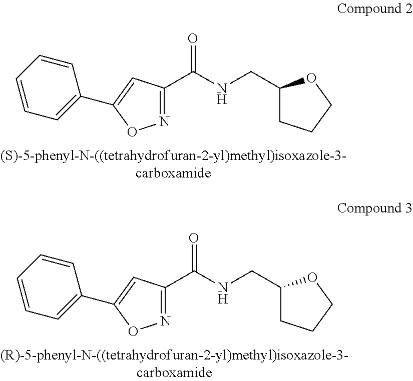 [SMILES: O=C(NC[C@@H]1CCCO1)c1cc(-c2ccccc2)on1 ; O=C(NC[C@H]1CCCO1)c1cc(-c2ccccc2)on1]

In additional embodiments, the invention is directed to Compounds 20, 90, 92, 115, 135, 188, 194, 195, 197, 198, 226, 230, 336, 349 and 376 shown in the Table below:

TABLE 1A

| Com- | |
|---|---|
| pound | |
| No. | |
| 20 | 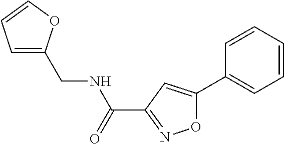 [SMILES: O=C(NCc1ccco1)c1cc(-c2ccccc2)on1] |
| 90 | 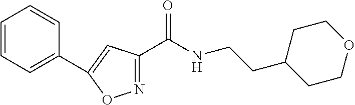 [SMILES: O=C(NCCC1CCOCC1)c1cc(-c2ccccc2)on1] |
| 92 | 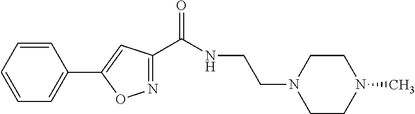 |
| 115 | 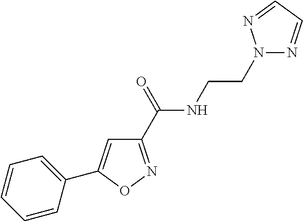 [SMILES: O=C(NCCn1nccn1)c1cc(-c2ccccc2)on1] |
| 135 | 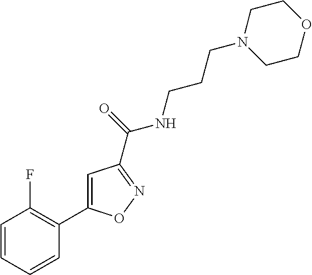 [SMILES: O=C(NCCCN1CCOCC1)c1cc(-c2ccccc2F)on1] |
| 188 | 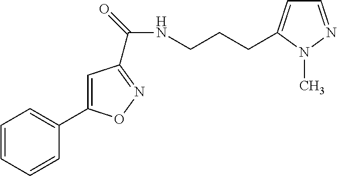 [SMILES: Cn1nccc1CCCNC(=O)c1cc(-c2ccccc2)on1] |
| 194 | 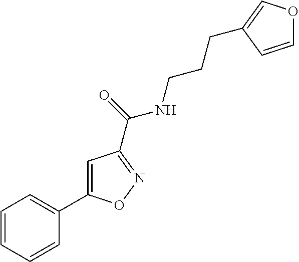 [SMILES: O=C(NCCCc1ccoc1)c1cc(-c2ccccc2)on1] |
| 195 | 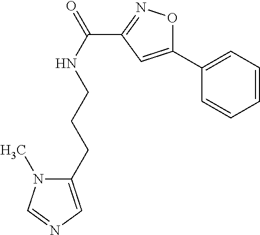 [SMILES: Cn1cncc1CCCNC(=O)c1cc(-c2ccccc2)on1] |
| 197 | 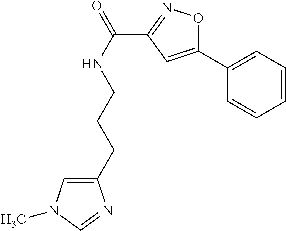 [SMILES: Cn1cnc(CCCNC(=O)c2cc(-c3ccccc3)on2)c1] |
| 198 | 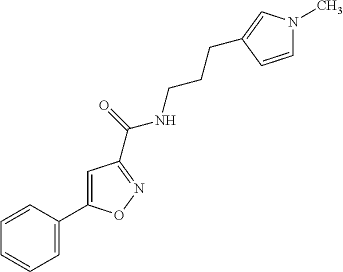 [SMILES: Cn1ccc(CCCNC(=O)c2cc(-c3ccccc3)on2)c1] |
| 226 | 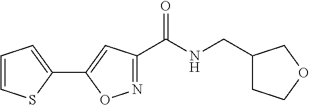 [SMILES: O=C(NCC1CCOC1)c1cc(-c2cccs2)on1] |
| 230 | 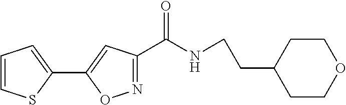 [SMILES: O=C(NCCC1CCOCC1)c1cc(-c2cccs2)on1] |
| 336 | 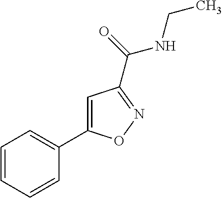 [SMILES: CCNC(=O)c1cc(-c2ccccc2)on1] |
| 349 | 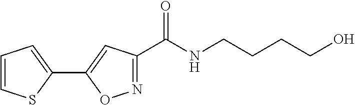 [SMILES: O=C(NCCCCO)c1cc(-c2cccs2)on1] |
| 376 | 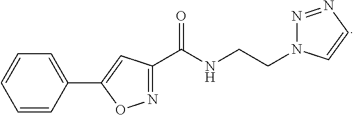 [SMILES: O=C(NCCn1ccnn1)c1cc(-c2ccccc2)on1] |

### DETAILED DESCRIPTION OF THE INVENTION

A description of preferred embodiments of the invention follows.

As used herein, the words “a” and “an” are meant to include one or more unless otherwise specified. For example, the term “a cell” encompasses both a single cell and a combination of two or more cells.

As discussed above, the present invention is directed to methods of modulating CFTR activity in a subject in need thereof comprising administering an effective amount of a compound of Formula (I), or a pharmaceutically acceptable salt, prodrug or solvate thereof. The invention also encompasses methods of treating a condition associated with CFTR activity or a disease associated with a dysfunction of proteostasis comprising administering to a subject an effective amount of a compound of Formula (I), or a pharmaceutically acceptable salt, prodrug or solvate thereof.

In some embodiments, the compound has the Formula (I), wherein R₁ is:

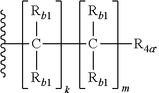

In an additional embodiment, the compound has the Formula (I), wherein R₁ is:

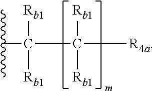

In additional embodiments, the compound has the Formula (I), wherein R₁ is:

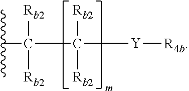

In yet additional embodiments, the compound has the Formula (I), wherein R₁ is

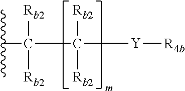

and m is 1.

In yet additional embodiments, the compound has the Formula (I), wherein R₁ is

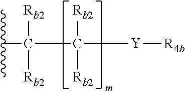

and Y is S(O)ₙ, O or NR_{d}.

In some aspects, the compound has the Formula (I) and m is 0, 1, 2, 3, 4 or 5. In additional aspects, the compound has the Formula (I) and m is 0, 1 or 2. In yet additional aspects, the compound has the Formula (I) and k is 1 and m is 0, 1 or 2.

In some embodiments, the compound has the Formula (I), wherein R₃ is hydrogen or optionally substituted C₁-C₁₀ alkyl. In additional embodiments, R₃ is hydrogen.

In yet further embodiments; the compound has the Formula (I), wherein Rₐ is hydrogen or optionally substituted C₁-C₄ alkyl. In yet other aspects, Rₐ is hydrogen.

In additional aspects of the invention, the compound has the Formula (I), wherein each of R_{b1} and R_{b2} is independently selected from hydrogen, OR_{c}, and optionally substituted C₁-C₁₀ alkyl, wherein R_{c} is hydrogen or optionally substituted C₁-C₁₀ alkyl.

In yet additional aspects, the compound has the Formula (I), wherein R₂ is selected from the group consisting of optionally substituted C₁-C₁₀ alkyl, optionally substituted C₃-C₁₂ cycloalkyl, optionally substituted C₃-C₁₂ cycloalkenyl, optionally substituted aryl, optionally substituted heterocyclic and optionally substituted heteroaryl. In yet further aspects, R₂ is selected from the group consisting of optionally substituted C₃-C₁₂ cycloalkyl, optionally substituted C₃-C₁₂ cycloalkenyl, optionally substituted aryl, optionally substituted heterocyclic and optionally substituted heteroaryl. In a further embodiment, R₂ is optionally substituted aryl. In some embodiments R₂ is optionally substituted phenyl. In certain embodiments, R₂ is unsubstituted phenyl. In some embodiments, R₂ is phenyl with a substitution at the para-position. In yet other aspects, R₂ is optionally substituted heteroaryl. In some embodiments, R₂ is optionally substituted thienyl, optionally substituted furanyl or optionally substituted pyridinyl. In certain embodiments, R₂ is optionally substituted thienyl.

In some embodiments, the compound has the Formula (I), wherein R₄ₐ is selected from the group consisting of optionally substituted C₁-C₁₀ alkyl, optionally substituted C₃-C₁₂ cycloalkyl, optionally substituted C₃-C₁₂ cycloalkenyl, optionally substituted aryl, OR_{c}, C(O)OR_{c}, C(O)R_{c}, C(O)C(O)R_{c}, C(O)NR_{d}R_{d}, optionally substituted heterocyclic and optionally substituted heteroaryl. In some embodiments, R₄ₐ is an optionally substituted aryl, optionally substituted heterocyclic or optionally substituted heteroaryl. In yet additional embodiments, R₄ₐ is an optionally substituted heterocyclic or optionally substituted heteroaryl. In some embodiments, R₄ₐ is cyclopentyl, tetrahydropyranyl, triazolyl, thiadiazolyl, oxazolidinonyl, tetrahydrofuranyl, oxazolinyl, piperazinyl or morpholinyl, each optionally substituted. In yet additional embodiments, R₄ₐ is 2-tetrahydrofuranyl or N-morpholinyl, each optionally substituted. In an additional embodiment, R₄ₐ is N-methyl piperazinyl. In yet further aspects, R₄ₐ is an optionally substituted heteroaryl containing one or more ring nitrogen atoms. In yet additional embodiments, R₄ₐ is selected from the group consisting of furanyl, pyridinyl, pyrazinyl, pyrazolyl, imidazolyl, isoxazolyl, triazolyl, thiazolyl, oxadiazolyl, thienyl, and benzimidazolyl, each optionally substituted. In some embodiments, R₄ₐ is optionally substituted 2-furanyl. In yet additional embodiments, R₄ₐ is C(O)NR_{d}R_{d}.

In some embodiments, the compound has the Formula (I) and k is 0. In yet an additional embodiment, k is 0 and R₄ₐ is an optionally substituted heterocyclic or an optionally substituted heteroaryl.

In certain additional embodiments, the compound has the Formula (I), wherein R₁ is

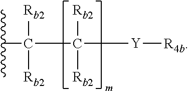

In some embodiments, Y is selected from the group consisting of S, S(O)₂ or S(O)₂NR_{d}, O and NR_{d}. In some embodiments, R_{4b} is selected from the group consisting of hydrogen, optionally substituted C₁-C₁₀ alkyl, optionally substituted C₃-C₁₂ cycloalkyl, optionally substituted C₃-C₁₂ cycloalkenyl, optionally substituted aryl, optionally substituted heteroaryl and optionally substituted heterocyclic. In yet additional embodiments, R_{4b} is optionally substituted C₁-C₁₀ alkyl, optionally substituted C₃-C₁₂ cycloalkyl, optionally substituted C₃-C₁₂ cycloalkenyl, optionally substituted aryl, optionally substituted heterocyclic and optionally substituted heteroaryl. In yet further embodiments, R_{4b} is an optionally substituted heterocyclic or optionally substituted heteroaryl. In some embodiments, R_{4b} is tetrahydropyranyl, tetrahydrofuranyl, or oxazolidinyl, each optionally substituted. In certain aspects, R_{4b} is optionally substituted 2-tetrahydrofuranyl. In yet additional embodiments, R_{4b} is an optionally substituted heteroaryl. In some embodiments, R_{4b} is selected from the group consisting of furanyl, pyridinyl, pyrazinyl, pyrazolyl, imidazolyl, isoxazolyl, triazolyl, thiazolyl, oxadiazolyl, thienyl, thiadiazolyl, and benzimidazolyl, each optionally substituted. In some embodiments, R_{4b} is optionally substituted furanyl or optionally substituted imidazolyl. In yet additional aspects, R_{4b} is a C₁-C₄ alkyl substituted with an optionally substituted heterocyclic or an optionally substituted heteroaryl, wherein said C₁-C₄ alkyl is optionally further substituted. In yet additional aspects, R_{4b} is a methyl or ethyl substituted with an optionally substituted heterocyclic or an optionally substituted heteroaryl, wherein said methyl or ethyl is optionally further substituted. In some embodiments, Y is S and S(O)₂. In additional embodiments, Y is S or S(O)₂ and R_{4b} is optionally substituted heterocyclic, optionally substituted heteroaryl, or C₁-C₄ alkyl substituted with an optionally substituted heterocyclic or an optionally substituted heteroaryl, wherein said C₁-C₄ alkyl is optionally further substituted. In additional embodiments, Y is O. In yet further aspects, Y is O and R_{4b} is optionally substituted C₁-C₁₀ alkyl, optionally substituted heterocyclic or optionally substituted heteroaryl. In some embodiments, Y is O and R_{4b} is optionally substituted C₁-C₄ alkyl.

In yet additional embodiments of the invention, the compound has the Formula (I), wherein R₂ is optionally substituted phenyl and R₄ₐ is an optionally substituted heterocyclic or optionally substituted heteroaryl. In additional embodiments, R₂ is optionally substituted phenyl, R₄ₐ is an optionally substituted heterocyclic or optionally substituted heteroaryl, R₃ is hydrogen and Rₐ is hydrogen or optionally substituted C₁-C₄ alkyl. In a further embodiment, R_{b1} is independently selected from hydrogen, OR_{c}, and optionally substituted C₁-C₁₀ alkyl, wherein Rₑ is C₁-C₁₀ alkyl.

In some embodiments of the invention, the compound has the Formula (I), wherein R₂ is unsubstituted phenyl and R₄ₐ is an optionally substituted heterocyclic or optionally substituted heteroaryl. Non-limiting examples of such compounds are shown below in Table 1. In additional embodiments, R₂ is unsubstituted phenyl, R₄ₐ is an optionally substituted heterocyclic or optionally substituted heteroaryl, R₃ is hydrogen and Rₐ is hydrogen or optionally substituted C₁-C₄ alkyl. In a further embodiment, R_{b1} is independently selected from hydrogen, ORₑ, and C₁-C₁₀ alkyl, wherein R_{c} is C₁-C₁₀ alkyl.

In further embodiments, the compound has the Formula (I), wherein Rₐ and the nitrogen atom to which it is attached is taken together with the adjacent C(R_{b1})(R_{b1}) or C(R_{b2})(R_{b2}) to form an optionally substituted, 4- to 12-membered heterocyclic ring containing one or more ring nitrogen atoms, wherein said heterocyclic ring optionally contains one or more ring heteroatoms selected from oxygen and sulfur. It will be understood that, in accordance with Formula (I), when Rₐ and the nitrogen atom to which it is attached is taken together with the adjacent C(R_{b1})(R_{b1}) or C(R_{b2})(R_{b2}) to form an optionally substituted, 4- to 12-membered heterocyclic ring, k is 1 and the optionally substituted 4- to 12-membered heterocyclic ring is attached to

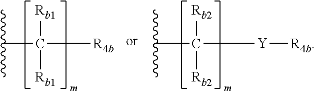

Non-limiting examples of such compounds are shown below in Table 19. In some embodiments, R₂ is an optionally substituted aryl, for example, optionally substituted phenyl. In yet additional aspects, R₄ₐ is selected from the group consisting of hydrogen, optionally substituted C₁-C₁₀ alkyl, ORₑ, C(O)NR_{d}, optionally substituted heteroaryl, and optionally substituted heterocyclic, wherein Rₑ is hydrogen or C₁-C₁₀ alkyl.

Exemplary compounds of Formula (I) and that can be used according to the methods of the invention are shown below in Table 1B.

TABLE 1B

| Compound No. | Chemical Structure |
|---|---|
| 1 | 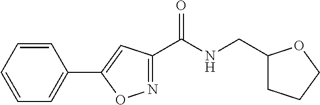 [SMILES: O=C(NCC1CCCO1)c1cc(-c2ccccc2)on1] |
| 2 | 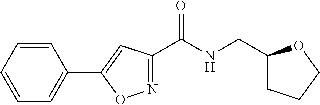 [SMILES: O=C(NC[C@@H]1CCCO1)c1cc(-c2ccccc2)on1] |
| | (5)-5-phenyl-N-((tetrahydrofuran-2-yl)methyl)isoxazole-3- |
| | carboxamide |
| 3 | 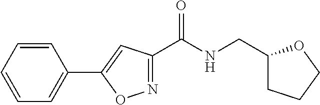 [SMILES: O=C(NC[C@H]1CCCO1)c1cc(-c2ccccc2)on1] |
| | (R)-5-phenyl-N-((tetrahydrofuran-2-yl)methyl)isoxazole-3- |
| | carboxamide |
| 4 | 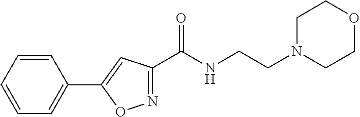 [SMILES: O=C(NCCN1CCOCC1)c1cc(-c2ccccc2)on1] |
| 5 | 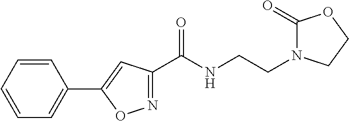 [SMILES: O=C(NCCN1CCOC1=O)c1cc(-c2ccccc2)on1] |
| 6 | 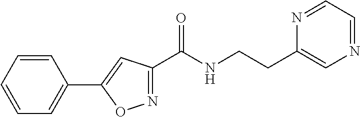 [SMILES: O=C(NCCc1cnccn1)c1cc(-c2ccccc2)on1] |
| 7 | 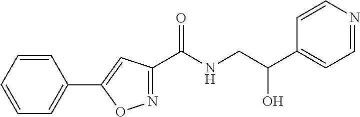 [SMILES: O=C(NCC(O)c1ccncc1)c1cc(-c2ccccc2)on1] |
| 8 | 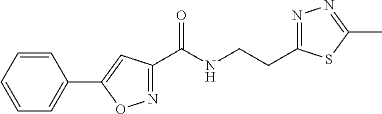 [SMILES: Cc1nnc(CCNC(=O)c2cc(-c3ccccc3)on2)s1] |
| 9 | 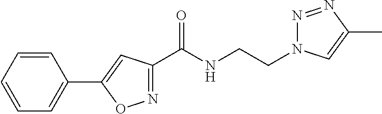 [SMILES: Cc1cn(CCNC(=O)c2cc(-c3ccccc3)on2)nn1] |
| 10 | 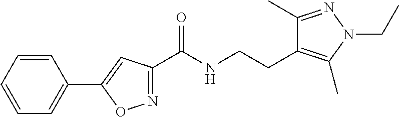 [SMILES: CCn1nc(C)c(CCNC(=O)c2cc(-c3ccccc3)on2)c1C] |
| 11 | 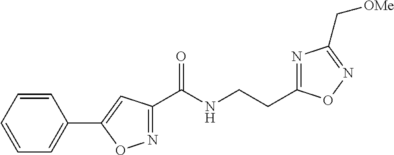 [SMILES: COCc1noc(CCNC(=O)c2cc(-c3ccccc3)on2)n1] |
| 12 | 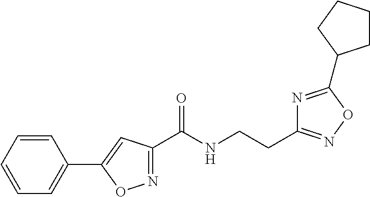 [SMILES: O=C(NCCc1noc(C2CCCC2)n1)c1cc(-c2ccccc2)on1] |
| 13 | 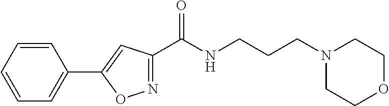 [SMILES: O=C(NCCCN1CCOCC1)c1cc(-c2ccccc2)on1] |
| 14 | 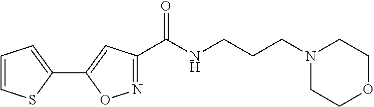 [SMILES: O=C(NCCCN1CCOCC1)c1cc(-c2cccs2)on1] |
| 15 | 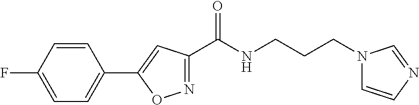 [SMILES: O=C(NCCCn1ccnc1)c1cc(-c2ccc(F)cc2)on1] |
| 16 | 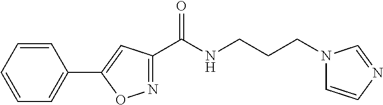 [SMILES: O=C(NCCCn1ccnc1)c1cc(-c2ccccc2)on1] |
| 17 | 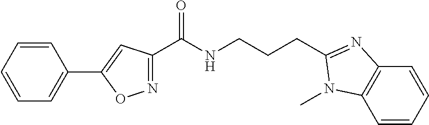 [SMILES: Cn1c(CCCNC(=O)c2cc(-c3ccccc3)on2)nc2ccccc21] |
| 18 | 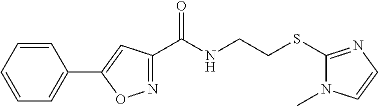 [SMILES: Cn1ccnc1SCCNC(=O)c1cc(-c2ccccc2)on1] |
| 19 | 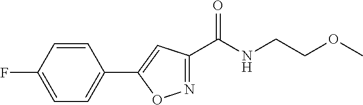 [SMILES: COCCNC(=O)c1cc(-c2ccc(F)cc2)on1] |

TABLE 2

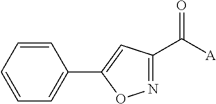

| Compound No. | A |
|---|---|
| 20 | 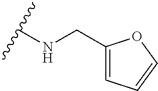 [SMILES: *NCc1ccco1] |
| 21 | 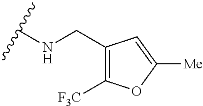 [SMILES: *NCc1cc(C)oc1C(F)(F)F] |
| 22 | 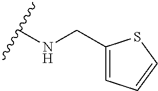 [SMILES: *NCc1cccs1] |
| 23 | 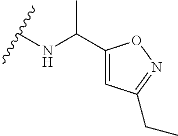 [SMILES: *NC(C)c1cc(CC)no1] |
| 24 | 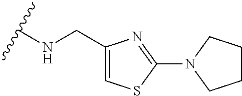 [SMILES: *NCc1csc(N2CCCC2)n1] |
| 25 | 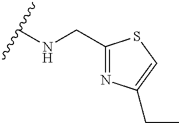 [SMILES: *NCc1nc(CC)cs1] |
| 26 | 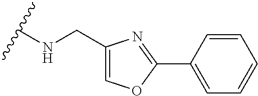 [SMILES: *NCc1coc(-c2ccccc2)n1] |
| 27 | 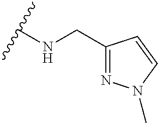 [SMILES: *NCc1ccn(C)n1] |
| 28 | 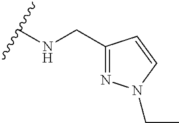 [SMILES: *NCc1ccn(CC)n1] |
| 29 | 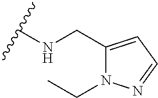 [SMILES: *NCc1ccnn1CC] |
| 30 | 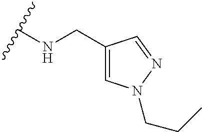 [SMILES: *NCc1cnn(CCC)c1] |
| 31 | 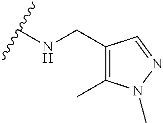 [SMILES: *NCc1cnn(C)c1C] |
| 32 | 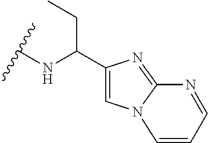 [SMILES: *NC(CC)c1cn2cccnc2n1] |
| 33 | 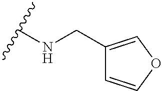 [SMILES: *NCc1ccoc1] |
| 34 | 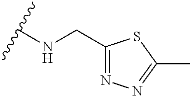 [SMILES: *NCc1nnc(C)s1] |
| 35 | 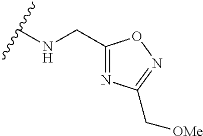 [SMILES: *NCc1nc(COC)no1] |
| 36 | 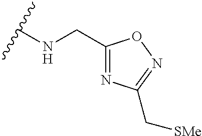 [SMILES: *NCc1nc(CSC)no1] |
| 37 | 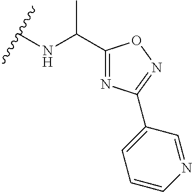 [SMILES: *NC(C)c1nc(-c2cccnc2)no1] |
| 38 | 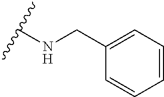 [SMILES: *NCc1ccccc1] |
| 39 | 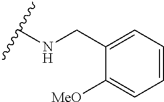 [SMILES: *NCc1ccccc1OC] |
| 40 | 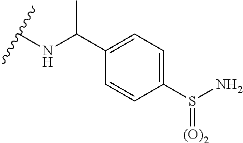 |
| 41 | 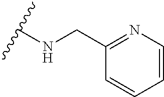 [SMILES: *NCc1ccccn1] |
| 42 | 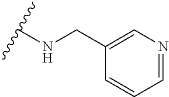 [SMILES: *NCc1cccnc1] |
| 43 | 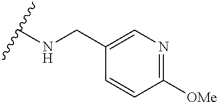 [SMILES: *NCc1ccc(OC)nc1] |
| 44 | 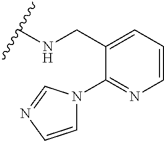 [SMILES: *NCc1cccnc1-n1ccnc1] |
| 45 | 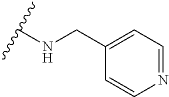 [SMILES: *NCc1ccncc1] |
| 46 | 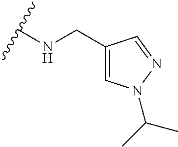 [SMILES: *NCc1cnn(C(C)C)c1] |
| 47 | 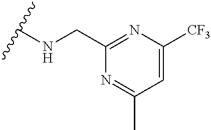 [SMILES: *NCc1nc(C)cc(C(F)(F)F)n1] |
| 48 | 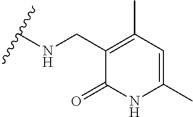 [SMILES: *NCc1c(C)cc(C)[nH]c1=O] |
| 49 | 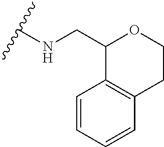 [SMILES: *NCC1OCCc2ccccc21] |
| 50 | 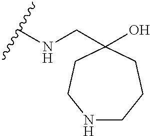 [SMILES: *NCC1(O)CCCNCC1] |
| 51 | 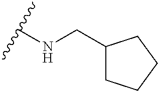 [SMILES: *NCC1CCCC1] |
| 52 | 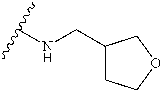 [SMILES: *NCC1CCOC1] |
| 53 | 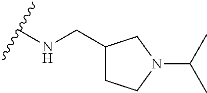 [SMILES: *NCC1CCN(C(C)C)C1] |
| 54 | 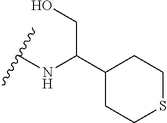 [SMILES: *NC(CO)C1CCSCC1] |
| 55 | 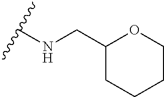 [SMILES: *NCC1CCCCO1] |
| 56 | 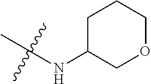 [SMILES: *NC1CCCOC1] |
| 57 | 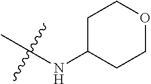 [SMILES: *NC1CCOCC1] |
| 58 | 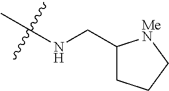 [SMILES: *NCC1CCCN1C] |
| 59 | 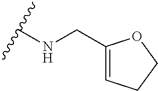 [SMILES: *NCC1=CCCO1] |
| 60 | 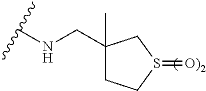 |

TABLE 3

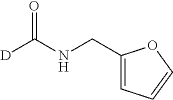

| Compound No. | D |
|---|---|
| 61 | 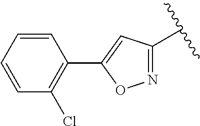 [SMILES: *c1cc(-c2ccccc2Cl)on1] |
| 62 | 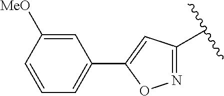 [SMILES: *c1cc(-c2cccc(OC)c2)on1] |
| 63 | 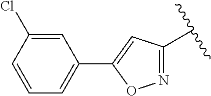 [SMILES: *c1cc(-c2cccc(Cl)c2)on1] |
| 64 | 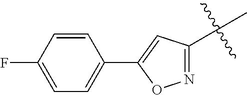 [SMILES: *c1cc(-c2ccc(F)cc2)on1] |
| 65 | 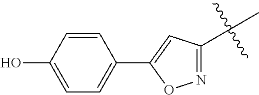 [SMILES: *c1cc(-c2ccc(O)cc2)on1] |
| 66 | 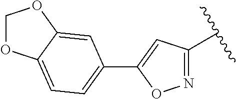 [SMILES: *c1cc(-c2ccc3c(c2)OCO3)on1] |
| 67 | 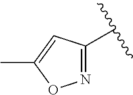 [SMILES: *c1cc(C)on1] |
| 68 | 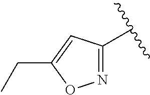 [SMILES: *c1cc(CC)on1] |
| 69 | 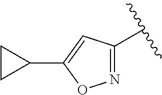 [SMILES: *c1cc(C2CC2)on1] |
| 70 | 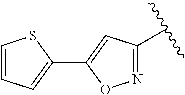 [SMILES: *c1cc(-c2cccs2)on1] |
| 71 | 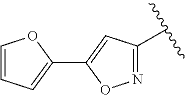 [SMILES: *c1cc(-c2ccco2)on1] |
| 72 | 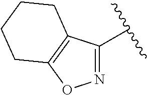 [SMILES: *c1noc2c1CCCC2] |

TABLE 4

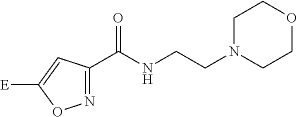

| Compound No. | E |
|---|---|
| 73 | 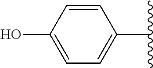 [SMILES: *c1ccc(O)cc1] |
| 74 | 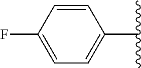 [SMILES: *c1ccc(F)cc1] |
| 75 | 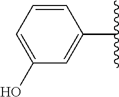 [SMILES: *c1cccc(O)c1] |
| 76 | 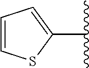 [SMILES: *c1cccs1] |
| 77 | 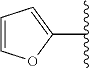 [SMILES: *c1ccco1] |

TABLE 5

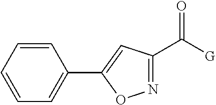

| Compound No. | G |
|---|---|
| 78 | 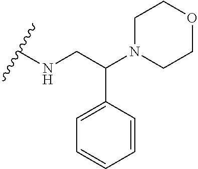 [SMILES: *NCC(c1ccccc1)N1CCOCC1] |
| 79 | 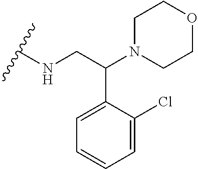 [SMILES: *NCC(c1ccccc1Cl)N1CCOCC1] |
| 80 | 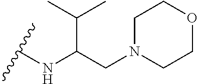 [SMILES: *NC(CN1CCOCC1)C(C)C] |
| 81 | 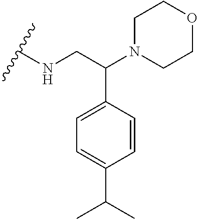 [SMILES: *NCC(c1ccc(C(C)C)cc1)N1CCOCC1] |
| 82 | 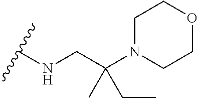 [SMILES: *NCC(C)(CC)N1CCOCC1] |

TABLE 6

| Compound No. | G′ |
|---|---|
| 83 | 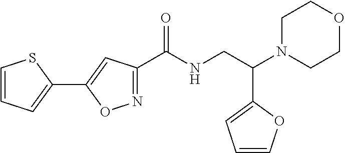 |
| 84 | 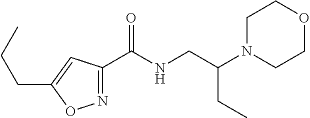 |
| 85 | 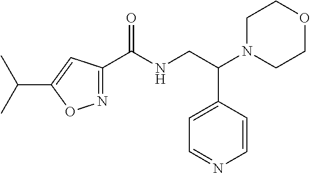 |
| 86 | 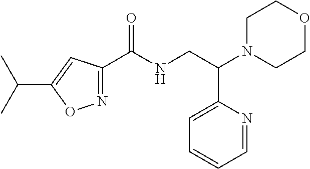 |

TABLE 7

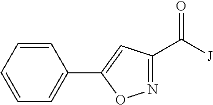

| Compound No. | J |
|---|---|
| 87 | 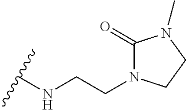 [SMILES: *NCCN1CCN(C)C1=O] |
| 88 | 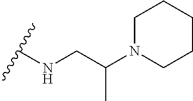 [SMILES: *NCC(C)N1CCCCC1] |
| 89 | 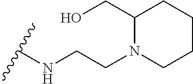 [SMILES: *NCCN1CCCCC1CO] |
| 90 | 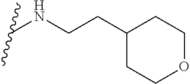 [SMILES: *NCCC1CCOCC1] |
| 91 | 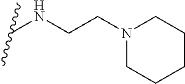 [SMILES: *NCCN1CCCCC1] |
| 92 | 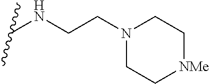 [SMILES: *NCCN1CCN(C)CC1] |
| 93 | 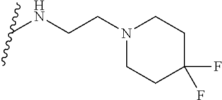 [SMILES: *NCCN1CCC(F)(F)CC1] |
| 94 | 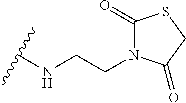 [SMILES: *NCCN1C(=O)CSC1=O] |
| 95 | 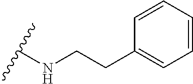 [SMILES: *NCCc1ccccc1] |
| 96 | 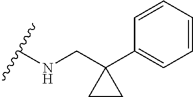 [SMILES: *NCC1(c2ccccc2)CC1] |
| 97 | 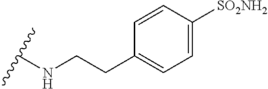 [SMILES: *NCCc1ccc(S(N)(=O)=O)cc1] |
| 98 | 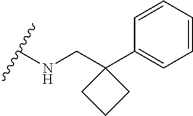 [SMILES: *NCC1(c2ccccc2)CCC1] |
| 99 | 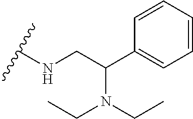 [SMILES: *NCC(c1ccccc1)N(CC)CC] |
| 100 | 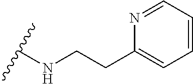 [SMILES: *NCCc1ccccn1] |
| 101 | 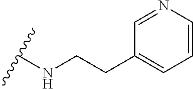 [SMILES: *NCCc1cccnc1] |
| 102 | 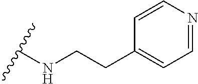 [SMILES: *NCCc1ccncc1] |
| 103 | 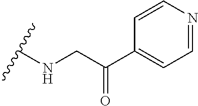 [SMILES: *NCC(=O)c1ccncc1] |
| 104 | 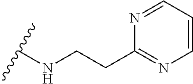 [SMILES: *NCCc1ncccn1] |
| 105 | 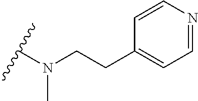 [SMILES: *N(C)CCc1ccncc1] |
| 106 | 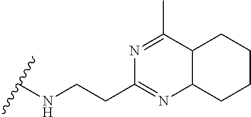 [SMILES: *NCCC1=NC2CCCCC2C(C)=N1] |
| 107 | 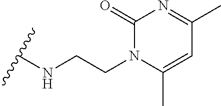 [SMILES: *NCCn1c(C)cc(C)nc1=O] |

TABLE 8

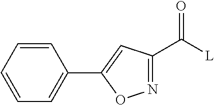

| Compound No. | L |
|---|---|
| 108 | 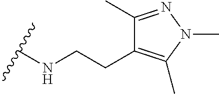 [SMILES: *NCCc1c(C)nn(C)c1C] |
| 109 | 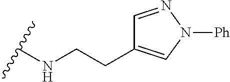 [SMILES: *NCCc1cnn(-c2ccccc2)c1] |
| 110 | 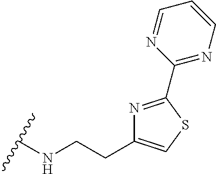 [SMILES: *NCCc1csc(-c2ncccn2)n1] |
| 111 | 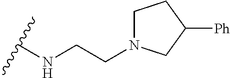 [SMILES: *NCCN1CCC(c2ccccc2)C1] |
| 112 | 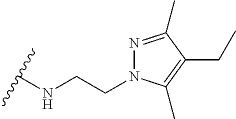 [SMILES: *NCCn1nc(C)c(CC)c1C] |
| 113 | 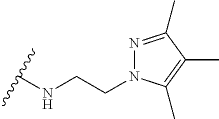 [SMILES: *NCCn1nc(C)c(C)c1C] |
| 114 | 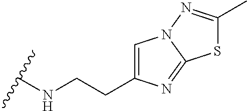 [SMILES: *NCCc1cn2nc(C)sc2n1] |
| 115 | 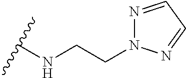 [SMILES: *NCCn1nccn1] |
| 116 | 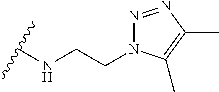 [SMILES: *NCCn1nnc(C)c1C] |
| 117 | 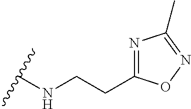 [SMILES: *NCCc1nc(C)no1] |
| 118 | 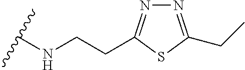 [SMILES: *NCCc1nnc(CC)s1] |
| 119 | 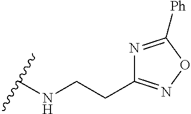 [SMILES: *NCCc1noc(-c2ccccc2)n1] |
| 120 | 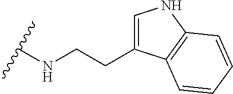 [SMILES: *NCCc1c[nH]c2ccccc12] |
| 121 | 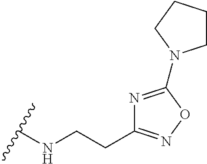 [SMILES: *NCCc1noc(N2CCCC2)n1] |
| 122 | 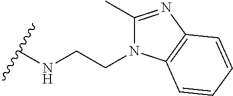 [SMILES: *NCCn1c(C)nc2ccccc21] |
| 123 | 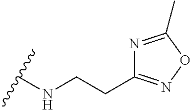 [SMILES: *NCCc1noc(C)n1] |

TABLE 9

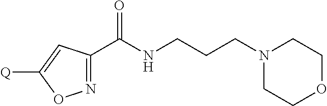

| Compound No. | Q |
|---|---|
| 124 | 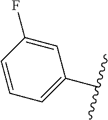 [SMILES: *c1cccc(F)c1] |
| 125 | 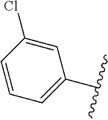 [SMILES: *c1cccc(Cl)c1] |
| 126 | 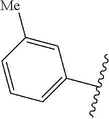 [SMILES: *c1cccc(C)c1] |
| 127 | 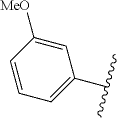 [SMILES: *c1cccc(OC)c1] |
| 128 | 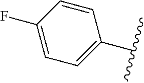 [SMILES: *c1ccc(F)cc1] |
| 129 | 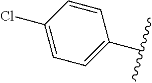 [SMILES: *c1ccc(Cl)cc1] |
| 130 | 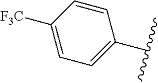 [SMILES: *c1ccc(C(F)(F)F)cc1] |
| 131 | 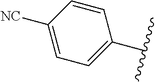 [SMILES: *c1ccc(C#N)cc1] |
| 132 | 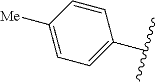 [SMILES: *c1ccc(C)cc1] |
| 133 | 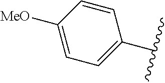 [SMILES: *c1ccc(OC)cc1] |
| 134 | 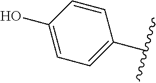 [SMILES: *c1ccc(O)cc1] |
| 135 | 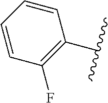 [SMILES: *c1ccccc1F] |
| 136 | 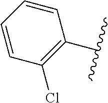 [SMILES: *c1ccccc1Cl] |
| 137 | 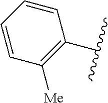 [SMILES: *c1ccccc1C] |
| 138 | 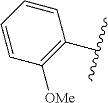 [SMILES: *c1ccccc1OC] |
| 139 | 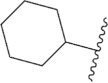 [SMILES: *C1CCCCC1] |
| 140 | 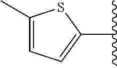 [SMILES: *c1ccc(C)s1] |
| 141 | 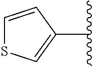 [SMILES: *c1ccsc1] |
| 142 | 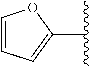 [SMILES: *c1ccco1] |

TABLE 10

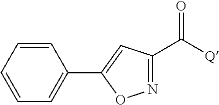

| Compound No. | Q′ |
|---|---|
| 143 | 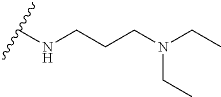 [SMILES: *NCCCN(CC)CC] |
| 144 | 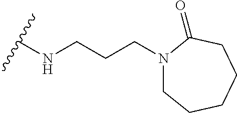 [SMILES: *NCCCN1CCCCCC1=O] |
| 145 | 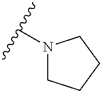 [SMILES: *N1CCCC1] |
| 146 | 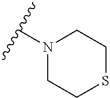 [SMILES: *N1CCSCC1] |
| 147 | 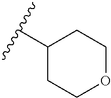 [SMILES: *C1CCOCC1] |
| 148 | 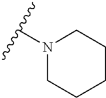 [SMILES: *N1CCCCC1] |
| 149 | 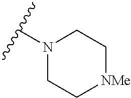 [SMILES: *N1CCN(C)CC1] |

TABLE 11

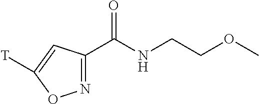

| Compound No. | T |
|---|---|
| 150 | 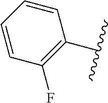 [SMILES: *c1ccccc1F] |
| 151 | 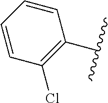 [SMILES: *c1ccccc1Cl] |
| 152 | 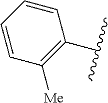 [SMILES: *c1ccccc1C] |
| 153 | 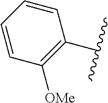 [SMILES: *c1ccccc1OC] |
| 154 | 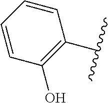 [SMILES: *c1ccccc1O] |
| 155 | 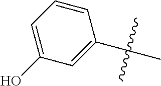 [SMILES: *c1cccc(O)c1] |
| 156 | 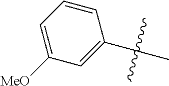 [SMILES: *c1cccc(OC)c1] |
| 157 | 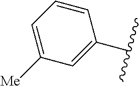 [SMILES: *c1cccc(C)c1] |
| 158 | 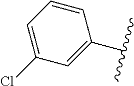 [SMILES: *c1cccc(Cl)c1] |
| 159 | 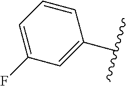 [SMILES: *c1cccc(F)c1] |
| 160 | 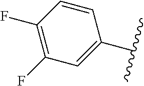 [SMILES: *c1ccc(F)c(F)c1] |
| 161 | 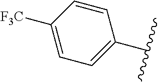 [SMILES: *c1ccc(C(F)(F)F)cc1] |
| 162 | 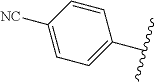 [SMILES: *c1ccc(C#N)cc1] |
| 163 | 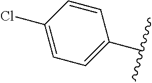 [SMILES: *c1ccc(Cl)cc1] |
| 164 | 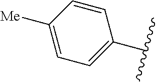 [SMILES: *c1ccc(C)cc1] |
| 165 | 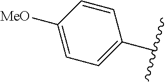 [SMILES: *c1ccc(OC)cc1] |
| 166 | 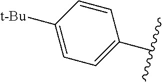 [SMILES: *c1ccc(C(C)(C)C)cc1] |
| 167 | 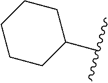 [SMILES: *C1CCCCC1] |
| 168 | 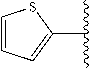 [SMILES: *c1cccs1] |
| 169 | 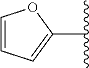 [SMILES: *c1ccco1] |
| 170 | 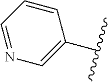 [SMILES: *c1cccnc1] |
| 171 | 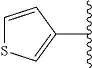 [SMILES: *c1ccsc1] |
| 329A | 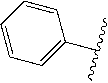 [SMILES: *c1ccccc1] |
| Compound 172 | 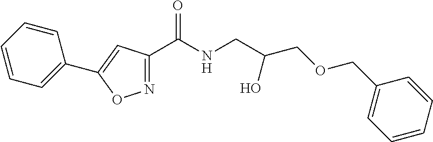 |
| Compound 173 | 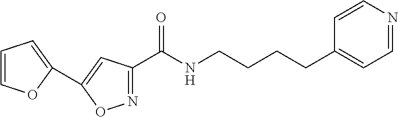 |

TABLE 12

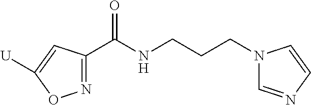

| Compound No. | U |
|---|---|
| 174 | 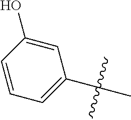 [SMILES: *c1cccc(O)c1] |
| 175 | 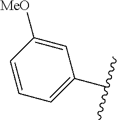 [SMILES: *c1cccc(OC)c1] |
| 176 | 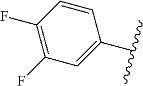 [SMILES: *c1ccc(F)c(F)c1] |
| 177 | 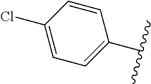 [SMILES: *c1ccc(Cl)cc1] |
| 178 | 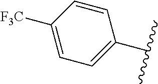 [SMILES: *c1ccc(C(F)(F)F)cc1] |
| 179 | 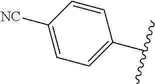 [SMILES: *c1ccc(C#N)cc1] |
| 180 | 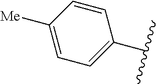 [SMILES: *c1ccc(C)cc1] |
| 181 | 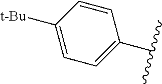 [SMILES: *c1ccc(C(C)(C)C)cc1] |
| 182 | 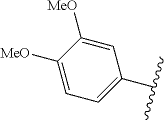 [SMILES: *c1ccc(OC)c(OC)c1] |
| 183 | 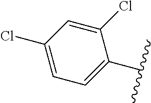 [SMILES: *c1ccc(Cl)cc1Cl] |
| 184 | 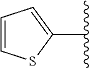 [SMILES: *c1cccs1] |
| 185 | 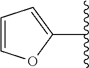 [SMILES: *c1ccco1] |

TABLE 13

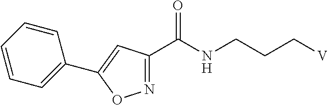

| Compound No. | V |
|---|---|
| 186 | 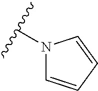 [SMILES: *n1cccc1] |
| 187 | 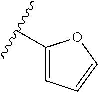 [SMILES: *c1ccco1] |
| 188 | 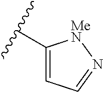 [SMILES: *c1ccnn1C] |
| 189 | 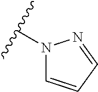 [SMILES: *n1cccn1] |
| 190 | 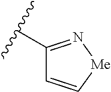 |
| 191 | 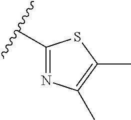 [SMILES: *c1nc(C)c(C)s1] |
| 192 | 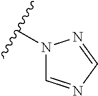 [SMILES: *n1cncn1] |
| 193 | 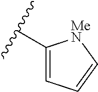 [SMILES: *c1cccn1C] |
| 194 | 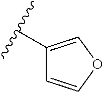 [SMILES: *c1ccoc1] |
| 195 | 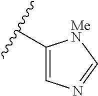 [SMILES: *c1cncn1C] |
| 196 | 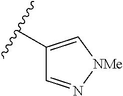 [SMILES: *c1cnn(C)c1] |
| 197 | 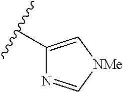 [SMILES: *c1cn(C)cn1] |
| 198 | 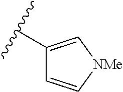 [SMILES: *c1ccn(C)c1] |
| 199 | 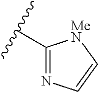 [SMILES: *c1nccn1C] |
| 200 | 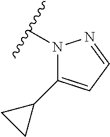 [SMILES: *n1nccc1C1CC1] |
| 201 | 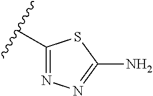 [SMILES: *c1nnc(N)s1] |
| 202 | 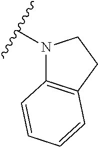 [SMILES: *N1CCc2ccccc21] |
| 203 | 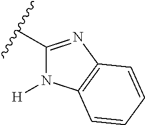 [SMILES: *c1nc2ccccc2[nH]1] |
| 204 | 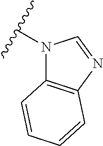 [SMILES: *n1cnc2ccccc21] |
| 205 | 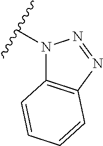 [SMILES: *n1nnc2ccccc21] |
| 206 | 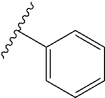 [SMILES: *c1ccccc1] |
| 207 | 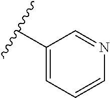 [SMILES: *c1cccnc1] |
| 208 | 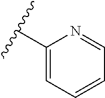 [SMILES: *c1ccccn1] |
| 209 | 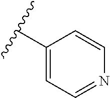 [SMILES: *c1ccncc1] |
| 210 | 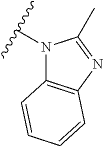 [SMILES: *n1c(C)nc2ccccc21] |
| 211 | 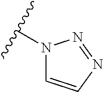 [SMILES: *n1ccnn1] |

TABLE 14

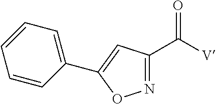

| Compound No. | V′ |
|---|---|
| 212 | 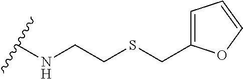 [SMILES: *NCCSCc1ccco1] |
| 213 | 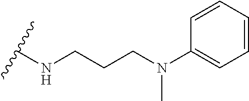 [SMILES: *NCCCN(C)c1ccccc1] |
| 214 | 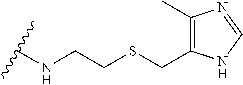 [SMILES: *NCCSCc1[nH]cnc1C] |
| 215 | 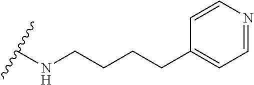 [SMILES: *NCCCCc1ccncc1] |
| 216 | 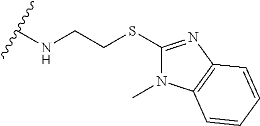 [SMILES: *NCCSc1nc2ccccc2n1C] |
| 217 | 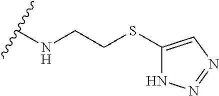 [SMILES: *NCCSc1cnn[nH]1] |
| 218 | 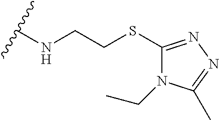 [SMILES: *NCCSc1nnc(C)n1CC] |
| 219 | 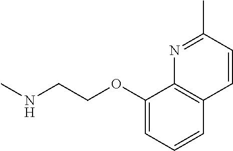 |
| 220 | 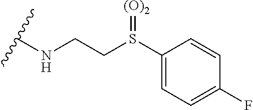 |
| 221 | 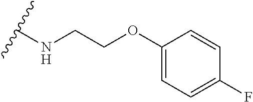 [SMILES: *NCCOc1ccc(F)cc1] |
| 222 | 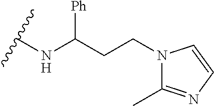 [SMILES: *NC(CCn1ccnc1C)c1ccccc1] |

TABLE 15

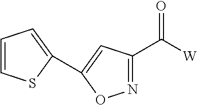

| Compound No. | W |
|---|---|
| 223 | 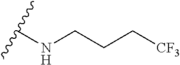 [SMILES: *NCCCC(F)(F)F] |
| 224 | 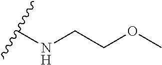 [SMILES: *NCCOC] |
| 225 | 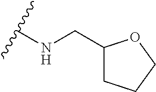 [SMILES: *NCC1CCCO1] |
| 226 | 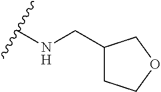 [SMILES: *NCC1CCOC1] |
| 227 | 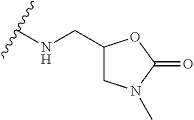 [SMILES: *NCC1CN(C)C(=O)O1] |
| 228 | 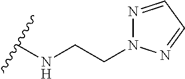 [SMILES: *NCCn1nccn1] |
| 229 | 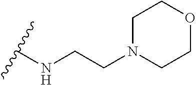 [SMILES: *NCCN1CCOCC1] |
| 230 | 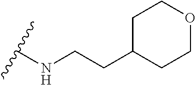 [SMILES: *NCCC1CCOCC1] |
| 231 | 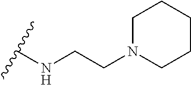 [SMILES: *NCCN1CCCCC1] |
| 232 | 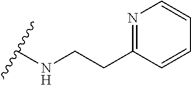 [SMILES: *NCCc1ccccn1] |
| 233 | 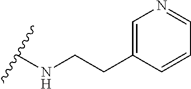 [SMILES: *NCCc1cccnc1] |
| 234 | 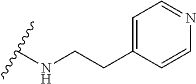 [SMILES: *NCCc1ccncc1] |
| 235 | 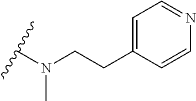 [SMILES: *N(C)CCc1ccncc1] |
| 236 | 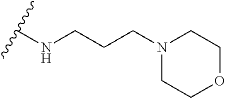 [SMILES: *NCCCN1CCOCC1] |
| 237 | 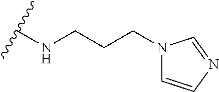 [SMILES: *NCCCn1ccnc1] |
| 238 | 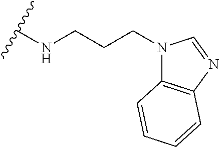 [SMILES: *NCCCn1cnc2ccccc21] |
| 239 | 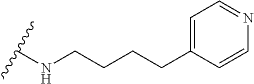 [SMILES: *NCCCCc1ccncc1] |
| 240 | 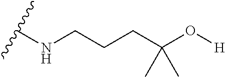 [SMILES: *NCCCC(C)(C)O] |

TABLE 16

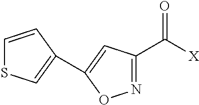

| Compound No. | X |
|---|---|
| 241 | 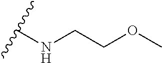 [SMILES: *NCCOC] |
| 242 | 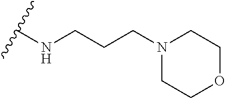 [SMILES: *NCCCN1CCOCC1] |

TABLE 17

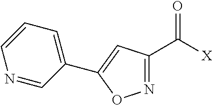

| Compound No. | Z |
|---|---|
| 243 | 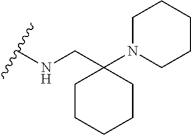 [SMILES: *NCC1(N2CCCCC2)CCCCC1] |
| 244 | 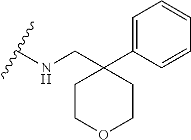 [SMILES: *NCC1(c2ccccc2)CCOCC1] |
| 245 | 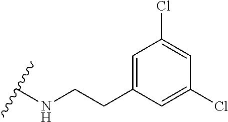 [SMILES: *NCCc1cc(Cl)cc(Cl)c1] |
| 246 | 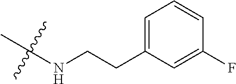 [SMILES: *NCCc1cccc(F)c1] |
| 247 | 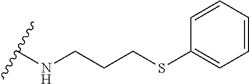 [SMILES: *NCCCSc1ccccc1] |
| 248 | 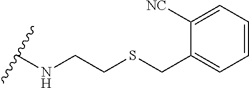 [SMILES: *NCCSCc1ccccc1C#N] |

TABLE 18

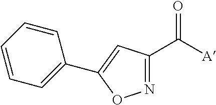

| Compound No. | A′ |
|---|---|
| 249 | 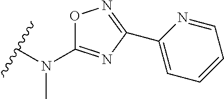 [SMILES: *N(C)c1nc(-c2ccccn2)no1] |
| 250 | 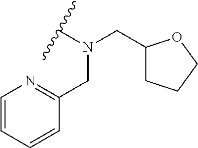 [SMILES: *N(Cc1ccccn1)CC1CCCO1] |
| 251 | 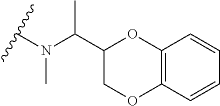 [SMILES: *N(C)C(C)C1COc2ccccc2O1] |
| 252 | 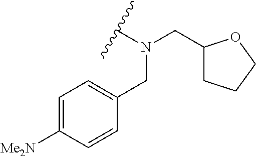 [SMILES: *N(Cc1ccc(N(C)C)cc1)CC1CCCO1] |
| 253 | 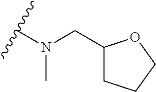 [SMILES: *N(C)CC1CCCO1] |
| 254 | 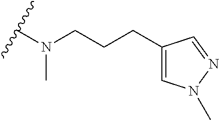 [SMILES: *N(C)CCCc1cnn(C)c1] |
| Compound 255 | 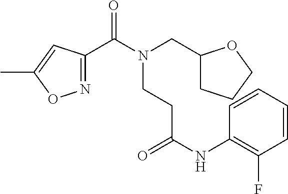 |
| Compound 256 | 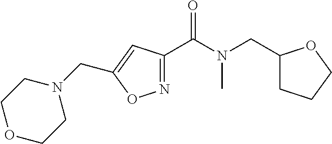 |
| Compound 257 | 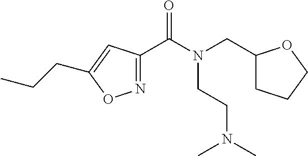 |

TABLE 19

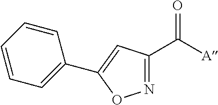

| Compound No. | A″ |
|---|---|
| 258 | 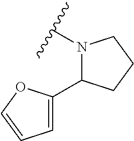 [SMILES: *N1CCCC1c1ccco1] |
| 259 | 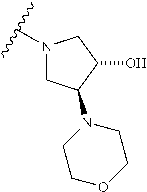 [SMILES: *N1C[C@H](O)[C@@H](N2CCOCC2)C1] |
| 260 | 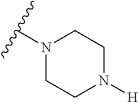 [SMILES: *N1CCNCC1] |
| 261 | 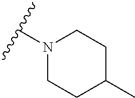 [SMILES: *N1CCC(C)CC1] |
| 262 | 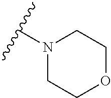 [SMILES: *N1CCOCC1] |
| 263 | 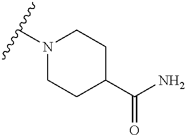 [SMILES: *N1CCC(C(N)=O)CC1] |
| 264 | 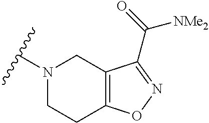 [SMILES: *N1CCc2onc(C(=O)N(C)C)c2C1] |
| 265 | 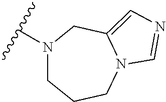 [SMILES: *N1CCCn2cncc2C1] |
| 266 | 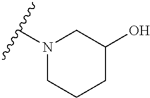 [SMILES: *N1CCCC(O)C1] |
| 267 | 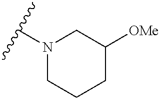 [SMILES: *N1CCCC(OC)C1] |
| Compound 268 | 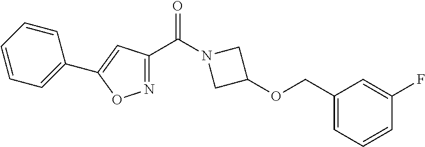 |
| Compound 269 | 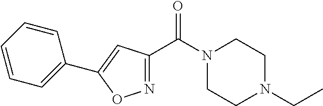 |

TABLE 20

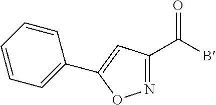

| Compound No. | B′ |
|---|---|
| 270 | 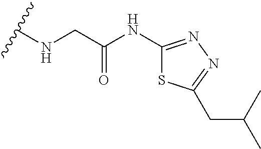 [SMILES: *NCC(=O)Nc1nnc(CC(C)C)s1] |
| 271 | 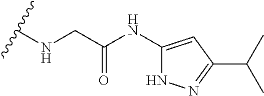 [SMILES: *NCC(=O)Nc1cc(C(C)C)n[nH]1] |
| 272 | 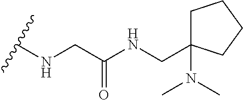 [SMILES: *NCC(=O)NCC1(N(C)C)CCCC1] |
| 273 | 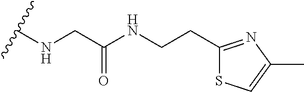 [SMILES: *NCC(=O)NCCc1nc(C)cs1] |
| 274 | 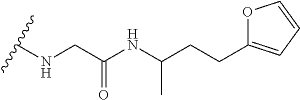 [SMILES: *NCC(=O)NC(C)CCc1ccco1] |
| 275 | 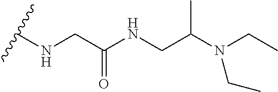 [SMILES: *NCC(=O)NCC(C)N(CC)CC] |
| 276 | 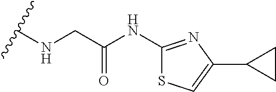 [SMILES: *NCC(=O)Nc1nc(C2CC2)cs1] |
| 277 | 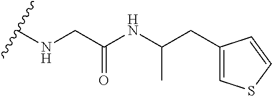 [SMILES: *NCC(=O)NC(C)Cc1ccsc1] |
| 278 | 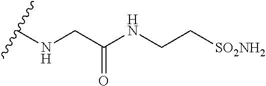 [SMILES: *NCC(=O)NCCS(N)(=O)=O] |
| 279 | 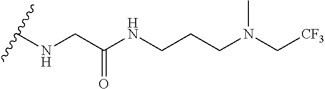 [SMILES: *NCC(=O)NCCCN(C)CC(F)(F)F] |
| 280 | 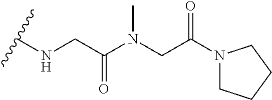 [SMILES: *NCC(=O)N(C)CC(=O)N1CCCC1] |
| 281 | 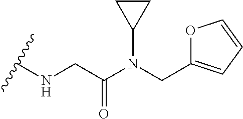 [SMILES: *NCC(=O)N(Cc1ccco1)C1CC1] |
| 282 | 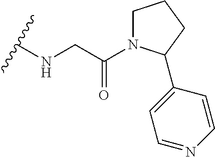 [SMILES: *NCC(=O)N1CCCC1c1ccncc1] |
| 283 | 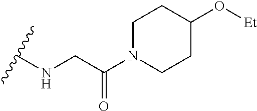 [SMILES: *NCC(=O)N1CCC(OCC)CC1] |
| 284 | 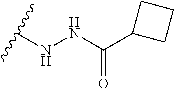 [SMILES: *NNC(=O)C1CCC1] |
| 285 | 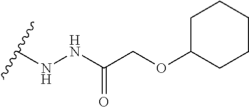 [SMILES: *NNC(=O)COC1CCCCC1] |
| 286 | 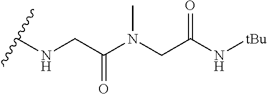 [SMILES: *NCC(=O)N(C)CC(=O)NC(C)(C)C] |
| 287 | 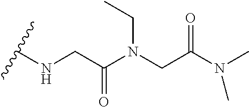 [SMILES: *NCC(=O)N(CC)CC(=O)N(C)C] |
| 288 | 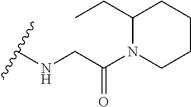 [SMILES: *NCC(=O)N1CCCCC1CC] |
| 289 | 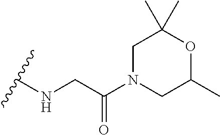 [SMILES: *NCC(=O)N1CC(C)OC(C)(C)C1] |
| 290 | 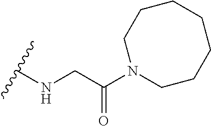 [SMILES: *NCC(=O)N1CCCCCCC1] |
| 291 | 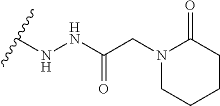 [SMILES: *NNC(=O)CN1CCCCC1=O] |
| 292 | 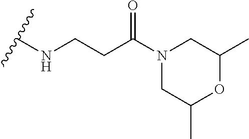 [SMILES: *NCCC(=O)N1CC(C)OC(C)C1] |

TABLE 21

| Com- | |
|---|---|
| pound No. | |
| 293 | 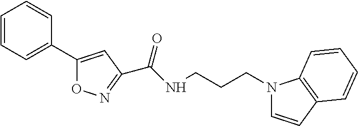 [SMILES: O=C(NCCCn1ccc2ccccc21)c1cc(-c2ccccc2)on1] |
| 294 | 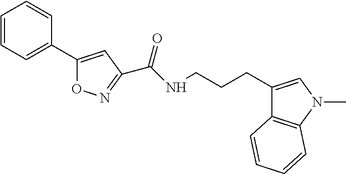 [SMILES: Cn1cc(CCCNC(=O)c2cc(-c3ccccc3)on2)c2ccccc21] |
| 295 | 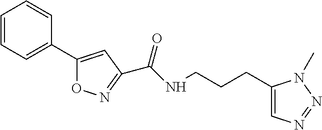 [SMILES: Cn1nncc1CCCNC(=O)c1cc(-c2ccccc2)on1] |
| 296 | 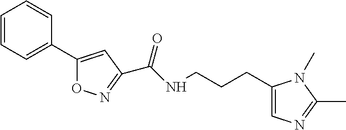 [SMILES: Cc1ncc(CCCNC(=O)c2cc(-c3ccccc3)on2)n1C] |
| 297 | 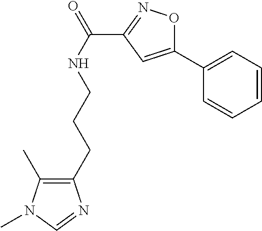 [SMILES: Cc1c(CCCNC(=O)c2cc(-c3ccccc3)on2)ncn1C] |
| 298 | 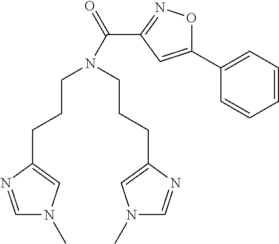 [SMILES: Cn1cnc(CCCN(CCCc2cn(C)cn2)C(=O)c2cc(-c3ccccc3)on2)c1] |
| 299 | 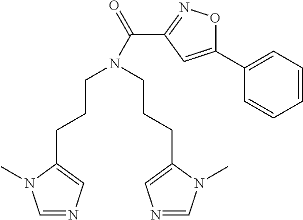 [SMILES: Cn1cncc1CCCN(CCCc1cncn1C)C(=O)c1cc(-c2ccccc2)on1] |
| 300 | 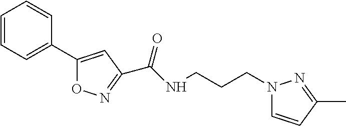 [SMILES: Cc1ccn(CCCNC(=O)c2cc(-c3ccccc3)on2)n1] |
| 301 | 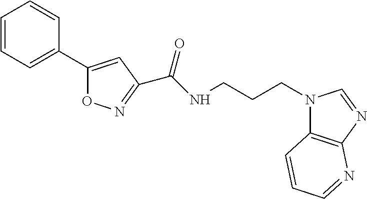 [SMILES: O=C(NCCCn1cnc2ncccc21)c1cc(-c2ccccc2)on1] |
| 302 | 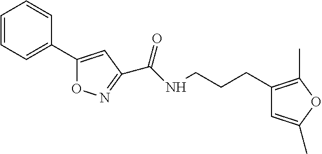 [SMILES: Cc1cc(CCCNC(=O)c2cc(-c3ccccc3)on2)c(C)o1] |
| 303 | 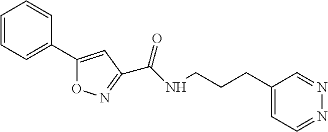 [SMILES: O=C(NCCCc1ccnnc1)c1cc(-c2ccccc2)on1] |

TABLE 22

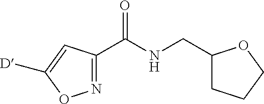

| Compound No. | D′ |
|---|---|
| 304 | 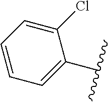 [SMILES: *c1ccccc1Cl] |
| 305 | 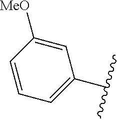 [SMILES: *c1cccc(OC)c1] |
| 306 | 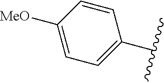 [SMILES: *c1ccc(OC)cc1] |
| 307 | 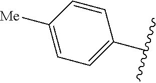 [SMILES: *c1ccc(C)cc1] |
| 308 | 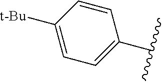 [SMILES: *c1ccc(C(C)(C)C)cc1] |
| 309 | 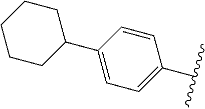 [SMILES: *c1ccc(C2CCCCC2)cc1] |
| 310 | 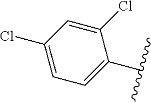 [SMILES: *c1ccc(Cl)cc1Cl] |
| 311 | H |
| 312 | Me |
| 313 | 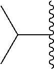 [SMILES: *C(C)C] |
| 314 | 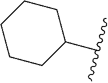 [SMILES: *C1CCCCC1] |
| 315 | 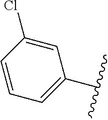 [SMILES: *c1cccc(Cl)c1] |
| 316 | 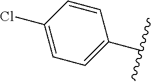 [SMILES: *c1ccc(Cl)cc1] |
| 317 | 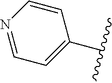 [SMILES: *c1ccncc1] |
| 318 | 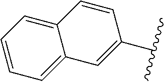 [SMILES: *c1ccc2ccccc2c1] |
| 319 | 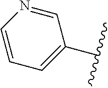 [SMILES: *c1cccnc1] |

TABLE 23

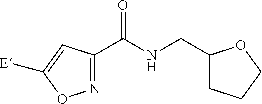

| Compound | |
|---|---|
| No. | E′ |
| 320 | 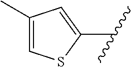 [SMILES: *c1cc(C)cs1] |
| 321 | 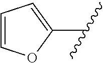 [SMILES: *c1ccco1] |
| 322 | 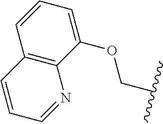 [SMILES: *COc1cccc2cccnc12] |
| 323 | 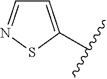 [SMILES: *c1ccns1] |
| 324 | 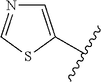 [SMILES: *c1cncs1] |
| 325 | 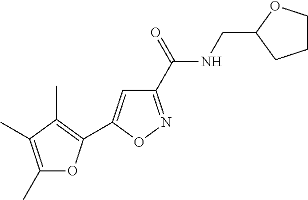 |
| 326 | 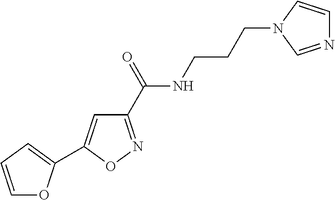 |
| 327 | 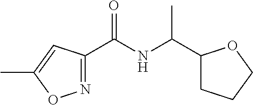 |
| 328 | 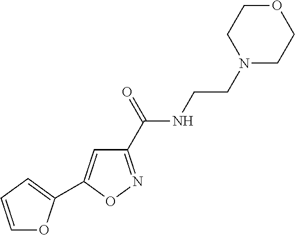 |

TABLE 24

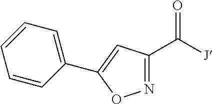

| Compound | |
|---|---|
| No. | J′ |
| 329B | 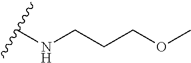 [SMILES: *NCCCOC] |
| 330 | 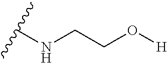 [SMILES: *NCCO] |
| 331 | 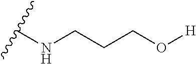 [SMILES: *NCCCO] |
| 332 | 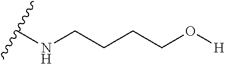 [SMILES: *NCCCCO] |
| 333 | 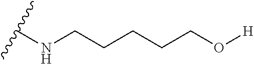 [SMILES: *NCCCCCO] |
| 334 | 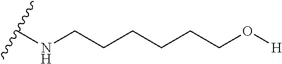 [SMILES: *NCCCCCCO] |
| 335 | 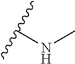 [SMILES: *NC] |
| 336 | 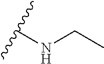 [SMILES: *NCC] |
| 337 | 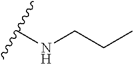 [SMILES: *NCCC] |
| 338 | 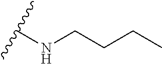 [SMILES: *NCCCC] |
| 339 | 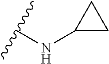 [SMILES: *NC1CC1] |
| 340 | 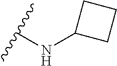 [SMILES: *NC1CCC1] |
| 341 | 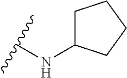 [SMILES: *NC1CCCC1] |
| Compound 342 | 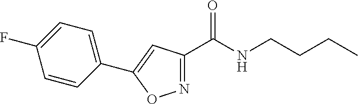 |
| Compound 343 | 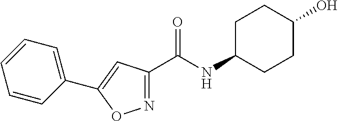 |
| Compound 344 | 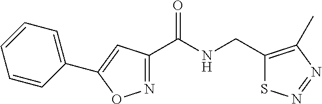 |
| Compound 345 | 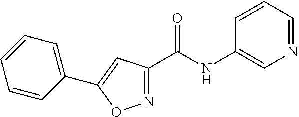 |

TABLE 25

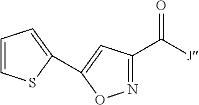

| Compound | |
|---|---|
| No. | J″ |
| 346 | 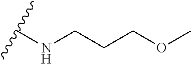 [SMILES: *NCCCOC] |
| 347 | 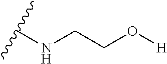 [SMILES: *NCCO] |
| 348 | 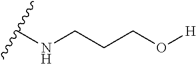 [SMILES: *NCCCO] |
| 349 | 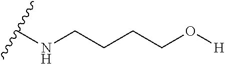 [SMILES: *NCCCCO] |
| 350 | 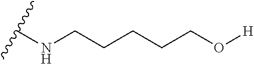 [SMILES: *NCCCCCO] |
| 351 | 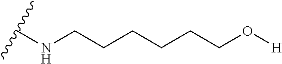 [SMILES: *NCCCCCCO] |
| 352 | 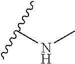 [SMILES: *NC] |
| 353 | 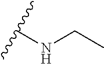 [SMILES: *NCC] |
| 354 | 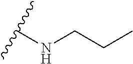 [SMILES: *NCCC] |
| 355 | 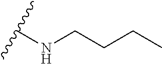 [SMILES: *NCCCC] |
| 356 | 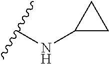 [SMILES: *NC1CC1] |
| 357 | 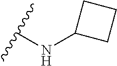 [SMILES: *NC1CCC1] |
| 358 | 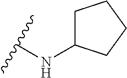 [SMILES: *NC1CCCC1] |

TABLE 26

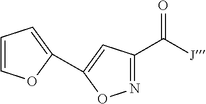

| Com- | |
|---|---|
| pound | |
| No. | J″″″ |
| 359 | 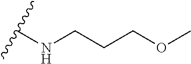 [SMILES: *NCCCOC] |
| 360 | 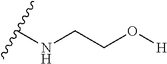 [SMILES: *NCCO] |
| 361 | 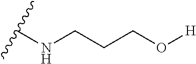 [SMILES: *NCCCO] |
| 362 | 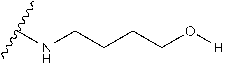 [SMILES: *NCCCCO] |
| 363 | 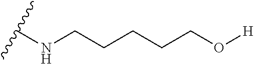 [SMILES: *NCCCCCO] |
| 364 | 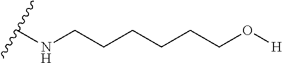 [SMILES: *NCCCCCCO] |
| 365 | 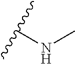 [SMILES: *NC] |
| 366 | 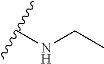 [SMILES: *NCC] |
| 367 | 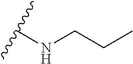 [SMILES: *NCCC] |
| 368 | 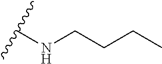 [SMILES: *NCCCC] |
| 369 | 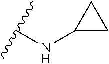 [SMILES: *NC1CC1] |
| 370 | 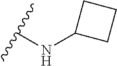 [SMILES: *NC1CCC1] |
| 371 | 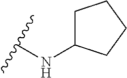 [SMILES: *NC1CCCC1] |
| Com- pound 372 | 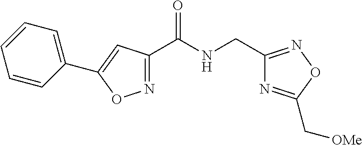 [SMILES: COCc1nc(CNC(=O)c2cc(-c3ccccc3)on2)no1] |
| Com- pound 373 | 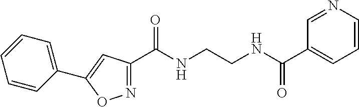 [SMILES: O=C(NCCNC(=O)c1cc(-c2ccccc2)on1)c1cccnc1] |
| Com- pound 374 | 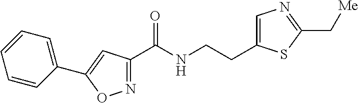 [SMILES: CCc1ncc(CCNC(=O)c2cc(-c3ccccc3)on2)s1] |
| Com- pound 375 | 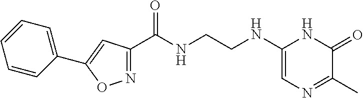 [SMILES: Cc1ncc(NCCNC(=O)c2cc(-c3ccccc3)on2)[nH]c1=O] |
| Com- pound 376 | 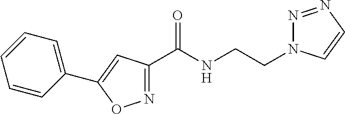 [SMILES: O=C(NCCn1ccnn1)c1cc(-c2ccccc2)on1] |
| Com- pound 377 | 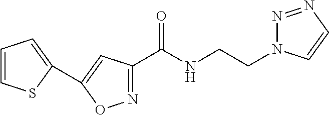 [SMILES: O=C(NCCn1ccnn1)c1cc(-c2cccs2)on1] |
| Com- pound 378 | 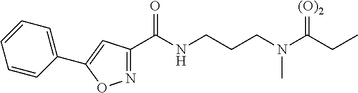 |

The invention also encompasses an enantiomerically pure compound having the structure below:

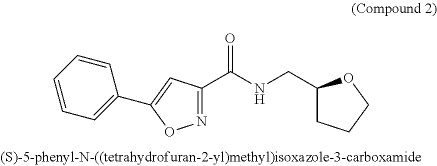 [SMILES: O=C(NC[C@@H]1CCCO1)c1cc(-c2ccccc2)on1]

The invention additionally encompasses an enantiomerically pure compound having the structure below:

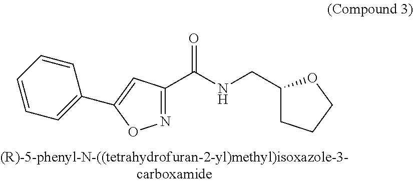 [SMILES: O=C(NC[C@H]1CCCO1)c1cc(-c2ccccc2)on1]

The invention also encompasses a compound selected from those shown below in Table 1A:

TABLE 1A

| Com- | |
|---|---|
| pound | |
| No. | |
| 20 | 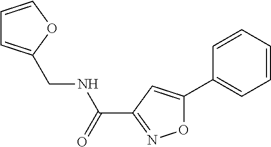 [SMILES: O=C(NCc1ccco1)c1cc(-c2ccccc2)on1] |
| 90 | 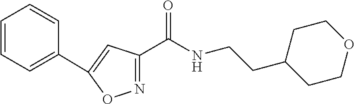 [SMILES: O=C(NCCC1CCOCC1)c1cc(-c2ccccc2)on1] |
| 92 | 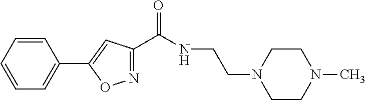 [SMILES: CN1CCN(CCNC(=O)c2cc(-c3ccccc3)on2)CC1] |
| 115 | 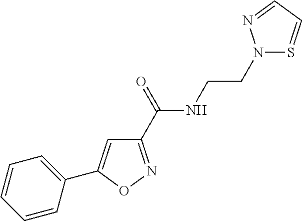 |
| 135 | 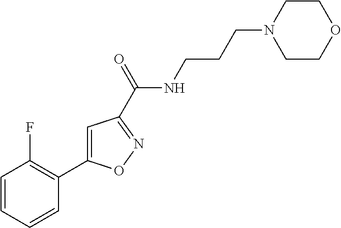 [SMILES: O=C(NCCCN1CCOCC1)c1cc(-c2ccccc2F)on1] |
| 188 | 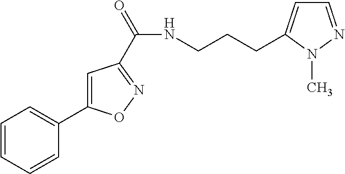 [SMILES: Cn1nccc1CCCNC(=O)c1cc(-c2ccccc2)on1] |
| 194 | 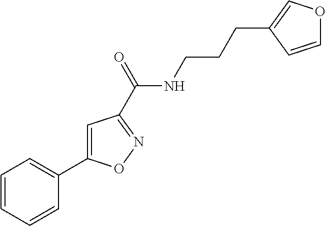 [SMILES: O=C(NCCCc1ccoc1)c1cc(-c2ccccc2)on1] |
| 195 | 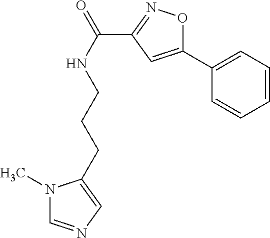 [SMILES: Cn1cncc1CCCNC(=O)c1cc(-c2ccccc2)on1] |
| 197 | 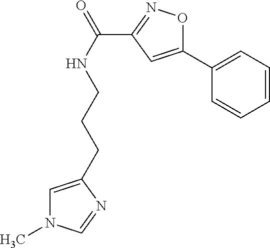 [SMILES: Cn1cnc(CCCNC(=O)c2cc(-c3ccccc3)on2)c1] |
| 198 | 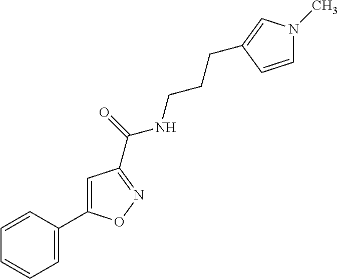 [SMILES: Cn1ccc(CCCNC(=O)c2cc(-c3ccccc3)on2)c1] |
| 226 | 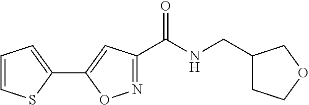 [SMILES: O=C(NCC1CCOC1)c1cc(-c2cccs2)on1] |
| 230 | 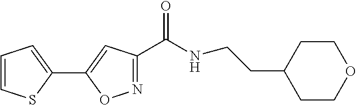 [SMILES: O=C(NCCC1CCOCC1)c1cc(-c2cccs2)on1] |
| 336 | 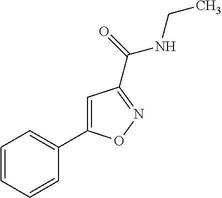 [SMILES: CCNC(=O)c1cc(-c2ccccc2)on1] |
| 349 | 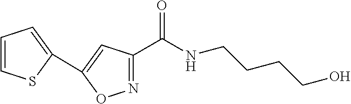 [SMILES: O=C(NCCCCO)c1cc(-c2cccs2)on1] |
| 376 | 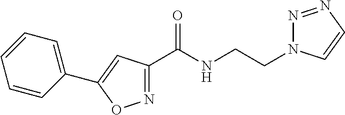 [SMILES: O=C(NCCn1ccnn1)c1cc(-c2ccccc2)on1] |

In some embodiments, the invention is a pharmaceutical composition comprising a pharmaceutically acceptable carrier and enantiomerically pure Compound 2. In additional embodiments, the invention is a pharmaceutical composition comprising a pharmaceutically acceptable carrier and an enantiomerically pure Compound 3.

In yet additional embodiments, the invention is a pharmaceutical composition comprising a compound selected from the group consisting of Compound 20, 90, 92, 115, 135, 188, 194, 195, 197, 198, 226, 230, 336, 349 and 376, and a pharmaceutically acceptable carrier.

It is to be understood that the specific embodiments described herein can be taken in combination with other specific embodiments delineated herein. For example, as discussed above, in some embodiments, R₂ is optionally substituted heteroaryl and in some embodiments described above, R₄ₐ is optionally substituted heterocyclic or optionally substituted heteroaryl. The invention thus encompasses compound of Formula (I) wherein R₂ is optionally substituted heteroaryl and R₄ₐ is optionally substituted heterocyclic or optionally substituted heteroaryl.

It will be appreciated that the description of the present invention herein should be construed in congruity with the laws and principals of chemical bonding.

The term “alkyl”, as used herein, unless otherwise indicated, refers to both branched and straight-chain saturated aliphatic hydrocarbon groups having the specified number of carbon atoms; for example, “C₁-C₁₀ alkyl” denotes alkyl having 1 to 10 carbon atoms. Examples of alkyl include, but are not limited to, methyl, ethyl, n-propyl, i-propyl, n-butyl, i-butyl, sec-butyl, t-butyl, n-pentyl, n-hexyl, 2-methylbutyl, 2-methylpentyl, 2-ethylbutyl, 3-methylpentyl, and 4-methylpentyl.

The term, “alkenyl”, as used herein, refers to both straight and branched-chain moieties having the specified number of carbon atoms and having at least one carbon-carbon double bond.

The term, “alkynyl”, as used herein, refers to both straight and branched-chain moieties having the specified number or carbon atoms and having at least one carbon-carbon triple bond.

The term “cycloalkyl,” as used herein, refers to cyclic alkyl moieties having 3 or more carbon atoms. Examples of cycloalkyl include, but are not limited to, cyclopropyl, cyclobutyl, cyclopentyl, cyclohexyl, cycloheptyl and adamantyl.

The term “cycloalkenyl,” as used herein, refers to cyclic alkenyl moieties having 3 or more carbon atoms.

The term “cycloalkynyl,” as used herein, refers to cyclic alkynyl moieties having 5 or more carbon atoms.

The term “heterocyclic” encompasses heterocycloalkyl, heterocycloalkenyl, heterobicycloalkyl, heterobicycloalkenyl, heteropolycycloalkyl, heteropolycycloalkenyl, and the like. Heterocycloalkyl refers to cycloalkyl groups containing one or more heteroatoms (O, S, or N) within the ring. Heterocycloalkenyl as used herein refers to cycloalkenyl groups containing one or more heteroatoms (O, S or N) within the ring. Heterobicycloalkyl refers to bicycloalkyl groups containing one or more heteroatoms (O, S or N) within a ring. Heterobicycloalkenyl as used herein refers to bicycloalkenyl groups containing one or more heteroatoms (O, S or N) within a ring.

Cycloalkyl, cycloalkenyl, heterocyclic, groups also include groups similar to those described above for each of these respective categories, but which are substituted with one or more oxo moieties.

The term “aryl”, as used herein, refers to mono- or polycyclic aromatic carbocyclic ring systems. A polycyclic aryl is a polycyclic ring system that comprises at least one aromatic ring. Polycyclic aryls can comprise fused rings, covalently attached rings or a combination thereof. The term “aryl” embraces aromatic radicals, such as, phenyl, naphthyl, indenyl, tetrahydronaphthyl, and indanyl. An aryl group may be substituted or unsubstituted. In some embodiments, the aryl is a C₄-C₁₀ aryl.

The term “heteroaryl”, as used herein, refers to aromatic carbocyclic groups containing one or more heteroatoms (O, S, or N) within a ring. A heteroaryl group can be monocyclic or polycyclic. A heteroaryl group may additionally be substituted or unsubstituted. The heteroaryl groups of this invention can also include ring systems substituted with one or more oxo moieties. A polycyclic heteroaryl can comprise fused rings, covalently attached rings or a combination thereof. A polycyclic heteroaryl is a polycyclic ring system that comprises at least one aromatic ring containing one or more heteroatoms within a ring. Polycyclic aryls can comprise fused rings, covalently attached rings or a combination thereof. Examples of heteroaryl groups include, but are not limited to, pyridinyl, pyridazinyl, imidazolyl, pyrimidinyl, pyrazolyl, triazolyl, pyrazinyl, quinolyl, isoquinolyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, triazinyl, isoindolyl, purinyl, oxadiazolyl, thiadiazolyl, furazanyl, benzofurazanyl, benzothiophenyl, benzotriazolyl, benzothiazolyl, benzoxazolyl, quinazolinyl, quinoxalinyl, naphthyridinyl, dihydroquinolyl, tetrahydroquinolyl, dihydroisoquinolyl, tetrahydroisoquinolyl, benzofuryl, furopyridinyl, pyrolopyrimidinyl, thiazolopyridinyl, oxazolopyridinyl and azaindolyl. The foregoing heteroaryl groups may be C-attached or heteroatom-attached (where such is possible). For instance, a group derived from pyrrole may be pyrrol-1-yl (N-attached) or pyrrol-3-yl (C-attached). In some embodiments, the heteroaryl is 4- to 10-membered heteroaryl.

The term “substituted” refers to substitution by independent replacement of one, two, or three or more of the hydrogen atoms with substituents including, but not limited to, —C₁-C₁₂ alkyl, —C₂-C₁₂ alkenyl, —C₂-C₁₂ alkynyl, —C₃-C₁₂ cycloalkyl, —C₃-C₁₂ cycloalkenyl, C₃-C₁₂ cycloalkynyl, -heterocyclic, —F, —Cl, —Br, —I, —OH, —NO₂, —N₃, —CN, —NH₂, oxo, thioxo, —NHRₓ, —NRₓRₓ, dialkylamino, -diarylamino, -diheteroarylamino, —ORₓ, —C(O)R_{y}, —C(O)C(O)R_{y}, —OCO₂R_{y}, —OC(O)R_{y}, OC(O)C(O)R_{y}, —NHC(O)R_{y}, —NHCO₂R_{y}, —NHC(O)C(O)R_{y}, NHC(S)NH₂, —NHC(S)NHRₓ, —NHC(NH)NH₂, —NHC(NH)NHRₓ, —NHC(NH)Rₓ, —C(NH)NHRₓ, and (C═NRₓ)Rₓ; —NRₓC(O)Rₓ, —NRₓC(O)N(Rₓ)₂, —NRₓCO₂R_{y}, —NRₓC(O)C(O)R_{y}, —NRₓC(S)NH₂, —NRₓC(S)NHRₓ, —NRₓC(NH)NH₂, —NRₓC(NH)NHRₓ, —NRₓC(NH)Rₓ, —C(NRₓ)NHRₓ—S(O)R_{y}, —NHSO₂Rₓ, —CH₂NH₂, —CH₂SO₂CH₃, -aryl, -arylalkyl, -heteroaryl, -heteroarylalkyl, - heterocycloalkyl, —C₃-C₁₂-cycloalkyl, -polyalkoxyalkyl, -polyalkoxy, -methoxymethoxy, -methoxyethoxy, —SH, —S—Rₓ, or -methylthiomethyl, wherein Rₓ is selected from the group consisting of hydrogen, —C₁-C₁₂ alkyl, —C₂-C₁₂ alkenyl, —C₂-C₁₂ alkynyl, —C₃-C₁₂ cycloalkyl, -aryl, -heteroaryl and -heterocyclic and —R_{y} is selected from the group consisting of hydrogen, —C₁-C₁₂ alkyl, —C₂-C₁₂ alkenyl, —C₂-C₁₂ alkynyl, —C₃-C₁₂ cycloalkyl, -aryl, -heteroaryl, -heterocyclic, —NH₂, —NH—C₁-C₁₂ alkyl, —NH—C₂-C₁₂ alkenyl, —NH—C₂-C₁₂-alkynyl, —NH—C₃-C₁₂ cycloalkyl, —NH-aryl, —NH— heteroaryl and —NH-heterocyclic. It is understood that the aryls, heteroaryls, alkyls, and the like can be further substituted.

The term “haloalkyl” as used herein refers to an alkyl group having 1 to (2n+1) substituent(s) independently selected from F, Cl, Br or I, where n is the maximum number of carbon atoms in the alkyl group.

As will be understood by the skilled artisan, “H” is the symbol for hydrogen, “N” is the symbol for nitrogen, “S” is the symbol for sulfur, “O” is the symbol for oxygen.

“Me” is an abbreviation for methyl.

Non-limiting examples of optionally substituted aryl are phenyl, substituted phenyl, napthyl and substituted naphthyl.

Certain of the compounds described herein contain one or more asymmetric centers and may thus give rise to enantiomers, diastereomers, and other stereoisomeric forms that may be defined, in terms of absolute stereochemistry, as (R)- or (S)-. The present invention is meant to include all such possible isomers, including racemic mixtures, optically pure forms and intermediate mixtures. Optically active (R)- and (S)-isomers may be prepared using chiral synthons or chiral reagents, or resolved using conventional techniques. “Isomers” are different compounds that have the same molecular formula. “Stereoisomers” are isomers that differ only in the way the atoms are arranged in space. “Enantiomers” are a pair of stereoisomers that are non-superimposable mirror images of each other. A 1:1 mixture of a pair of enantiomers is a “racemic” mixture. The term “(±)” is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. When the compounds described herein contain olefinic double bonds or other centers of geometric asymmetry, and unless specified otherwise, it is intended that the compounds include both E and Z geometric isomers. Likewise, all tautomeric forms are also intended to be included.

The term “enantiomerically pure” means a stereomerically pure composition of a compound. For example, a stereochemically pure composition is a composition that is free or substantially free of other stereoisomers of that compound. In another example, for a compound having one chiral center, an enantiomerically pure composition of the compound is free or substantially free of the other enantiomer. In yet another example, for a compound having two chiral centers, an enantiomerically pure composition is free or substantially free of the other diastereomers.

Where a particular stereochemistry is described or depicted it is intended to mean that a particular enantiomer is present in excess relative to the other enantiomer. A compound has an R-configuration at a specific position when it is present in excess compared to the compound having an S-configuration at that position. A compound has an S-configuration at a specific position when it is present in excess compared to the compound having an R-configuration at that position.

Likewise, all tautomeric forms are also intended to be included. Where a particular compound is described or depicted, it is intended to encompass that chemical structure as well as tautomers of that structure.

It is to be understood that atoms making up the compounds of the present invention are intended to include isotopic forms of such atoms. Isotopes, as used herein, include those atoms having the same atomic number but different mass numbers. Isotopes of hydrogen include, for example, tritium and deuterium, and isotopes of carbon include, for example, ¹³C and ¹⁴C. The invention therefore encompasses embodiments in which one or more of the hydrogen atoms in Formula (I) are replaced with deuterium. The invention also encompasses embodiments wherein one or more of the carbon atoms in Formula (I) is replaced with silicon atoms.

The invention additionally encompasses embodiment wherein one or more of the nitrogen atoms in Formula (I) are oxidized to N-oxide.

An exemplary synthetic route for the preparation of compound of Formula (I) that can be used according to the invention is shown in the schemes below. As will be understood by the skilled artisan, diastereomers can be separated from the reaction mixture using column chromatography.

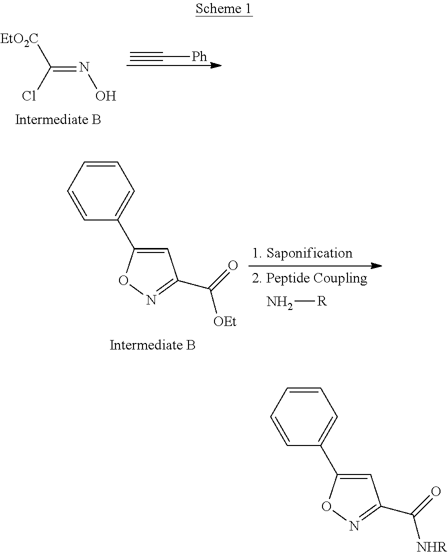

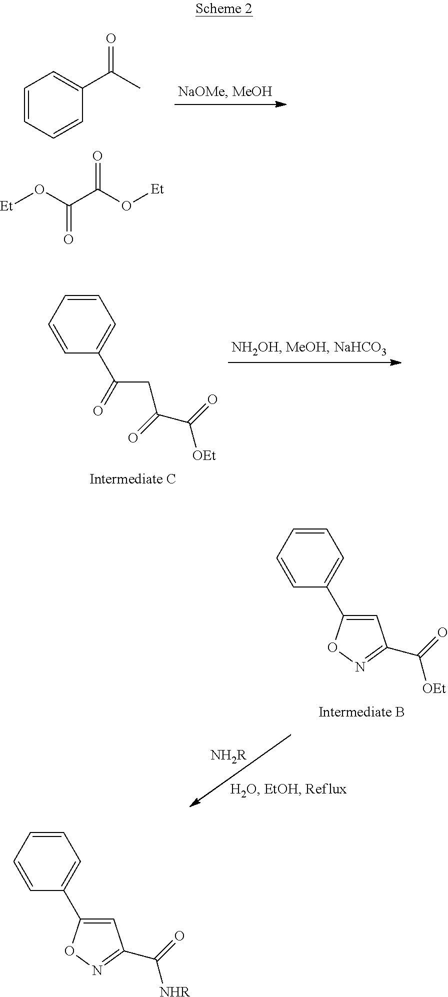

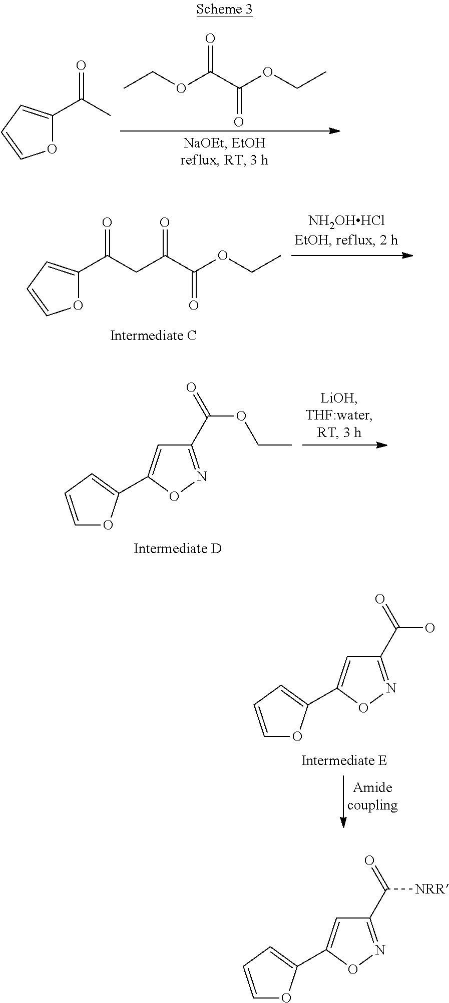

Compounds that can be used according to the methods of the invention can also be prepared using methods described in the literature, including, but not limited to, J. Med. Chem. 2011, 54(13), 4350-64; ChemMedChem. 2010, 5(10), 1667-1672; ChemMedChem. 2011, 6(8), 1363-1370; Russian Journal of Organic Chemistry, 2011, 47(8), 1199-1203; U.S. Patent Application Publication No. 2009/0036451 A1; WO2008/046072 A2, and U.S. Pat. No. 4,336,264, the contents of each of which are expressly incorporated by reference herein.

As discussed above, the invention is directed to a method of modulating CFTR activity in a subject comprising administering a compound of the invention in an effective amount. The invention also encompasses a method of treating a patient suffering from a condition associated with CFTR activity comprising administering to said patient a therapeutically effective amount of a compound described herein.

“Treating” or “treatment” includes preventing or delaying the onset of the symptoms, complications, or biochemical indicia of a disease, alleviating or ameliorating the symptoms or arresting or inhibiting further development of the disease, condition, or disorder. A “subject” is an animal to be treated or in need of treatment. A “patient” is a human subject in need of treatment.

An “effective amount” refers to that amount of an agent that is sufficient to achieve a desired and/or recited effect. In the context of a method of treatment, an “effective amount” of the therapeutic agent that is sufficient to ameliorate of one or more symptoms of a disorder and/or prevent advancement of a disorder, cause regression of the disorder and/or to achieve a desired effect.

The term “modulating” encompasses increasing, enhancing, inhibiting, decreasing, suppressing, and the like. As used herein, the terms “inhibiting” and “decreasing” encompass causing a net decrease by either direct or indirect means. The terms “increasing” and “enhancing” mean to cause a net gain by either direct or indirect means.

In some examples, CFTR activity is enhanced after administration of a compound described herein when there is an increase in the CFTR activity as compared to that in the absence of the compound. In some examples, CFTR activity is suppressed after administration of a compound described herein when there is a decrease in the CFTR activity as compared to that in the absence of the compound administration. CFTR activity encompasses, for example, chloride channel activity of the CFTR, and/or other ion transport activity (for example, HCO₃⁻ transport). Of the more than 1000 known mutations of the CFTR gene, ΔF508 is the most prevalent mutation of CFTR which results in misfolding of the protein and impaired trafficking from the endoplasmic reticulum to the apical membrane (Dormer et al. (2001). J Cell Sci 114, 4073-4081; http://www.genet.sickkids.on.ca/app). An enhancement or suppression of CFTR activity can be measured, for example, using literature described methods, including for example, Ussing chamber assays, patch clamp assays, and hBE Ieq assay (Devor et al. (2000), Am J Physiol Cell Physiol 279(2): C461-79; Dousmanis et al. (2002), J Gen Physiol 119(6): 545-59; Bruscia et al. (2005), PNAS 103(8): 2965-2971).

As discussed above, the invention also encompasses a method of treating cystic fibrosis. The present invention can also be used to treat other conditions associated with CFTR activity, including conditions associated with deficient CFTR activity and conditions that can be ameliorated by decreasing CFTR activity.

In some embodiments, the invention is directed to a method of treating a condition associated with deficient or decreased CFTR activity comprising administering an effective amount of a compound of Formula (I) that enhances CFTR activity. Non-limiting examples of conditions associated with deficient CFTR activity are cystic fibrosis, congenital bilateral absence of vas deferens (CBAVD), acute, recurrent, or chronic pancreatitis, disseminated bronchiectasis, asthma, allergic pulmonary aspergillosis, smoking-related lung diseases, such as chronic obstructive pulmonary disease (COPD), chronic sinusitis, dry eye disease, protein C deficiency, Aβ-lipoproteinemia, lysosomal storage disease, type 1 chylomicronemia, mild pulmonary disease, lipid processing deficiencies, type 1 hereditary angioedema, coagulation-fibrinolyis, hereditary hemochromatosis, CFTR-related metabolic syndrome, chronic bronchitis, constipation, pancreatic insufficiency, hereditary emphysema, and Sjogren's syndrome.

Methods of suppressing CFTR activity have been described as useful in treating conditions such as cholera and other secretory diarrheas, and polycystic kidney disease (Thiagarajah et al. (2012), Clin Pharmacol Ther 92(3): 287-90; Ma et al. (2002), J Clin Invest 110(11):1651-8; Yang et al. (2008), J Am Soc Nephrol. 19(7): 1300-1310). Thus, the invention encompasses methods of treating conditions that can be ameliorated by decreasing CFTR activity comprising administering an effective amount of a compound of Formula (I) that suppresses CFTR activity. Non-limiting examples of conditions that can be ameliorated by suppressing CFTR activity are cholera and other secretory diarrheas, and polycystic kidney disease.

In some embodiments, the methods of the invention further comprise administering an additional therapeutic agent. In additional embodiments, the invention encompasses a method of administering a compound of Formula (I), or a compound described herein, and at least one additional therapeutic agent. In certain aspects, the invention is directed to a method comprising administering a compound of Formula (I), or a compound described herein, and at least two additional therapeutic agents. Additional therapeutic agents include, for example, mucolytic agents, bronchodilators, antibiotics, anti-infective agents, anti-inflammatory agents, ion channel modulating agents, therapeutic agents used in gene therapy, CFTR correctors, and CFTR potentiators, or other agents that modulates CFTR activity. In some embodiments, at least one additional therapeutic agent is selected from the group consisting of a CFTR corrector and a CFTR potentiator. Non-limiting examples of CFTR correctors and potentiators are VX-770 (Ivacaftor), VX-809 (3-(6-(1-(2,2-difluorobenzo[d][1,3]dioxol-5-yl)cyclopropanecarboxamido)-3-methylpyridin-2-yl)benzoic acid, VX-661 (1-(2,2-difluoro-1,3-benzodioxol-5-yl)-N-[1-[(2R)-2,3-dihydroxypropyl]-6-fluoro-2-(2-hydroxy-1,1-dimethylethyl)-1H-indol-5-yl]-cyclopropanecarboxamide), VX-983, and Ataluren (PTC124) (3-[5-(2-fluorophenyl)-1,2,4-oxadiazol-3-yl]benzoic acid). Non-limiting examples of anti-inflammatory agents are N6022 (3-(5-(4-(1H-imidazol-1-yl) phenyl)-1-(4-carbamoyl-2-methylphenyl)-¹H-pyrrol-2-yl) propanoic acid), and N91115.

The invention encompasses administration of pharmaceutically acceptable salts of the compounds described herein. Thus, in certain aspects, the invention is directed to use of pharmaceutically acceptable salts of compounds of the invention and pharmaceutical compositions thereof. A “pharmaceutically acceptable salt” includes an ionic bond-containing product of the reaction between the disclosed compound with either an acid or a base, suitable for administering to a subject. Pharmaceutically acceptable salts are well known in the art and are described, for example, in Berge et al. (1977), Pharmaceutical Salts. Journal of Pharmaceutical Sciences, 69(1): 1-19, the contents of which are herein incorporated by reference. A non-limiting example of a pharmaceutically acceptable salt is an acid salt of a compound containing an amine or other basic group which can be obtained by reacting the compound with a suitable organic or inorganic acid. Examples of pharmaceutically acceptable salts also can be metallic salts including, but not limited to, sodium, magnesium, calcium, lithium and aluminum salts. Further examples of pharmaceutically acceptable salts include hydrochlorides, hydrobromides, sulfates, methanesulfonates, nitrates, maleates, acetates, citrates, fumarates, tartrates (e.g. (+)-tartrates, (−)-tartrates or mixtures thereof including racemic mixtures), succinates, benzoates and salts with amino acids such as glutamic acid. Salts can also be formed with suitable organic bases when the compound comprises an acid functional group such as —C(O)OH or —SO₃H. Such bases suitable for the formation of a pharmaceutically acceptable base addition salts with compounds of the present invention include organic bases that are nontoxic and strong enough to react with the acid functional group. Such organic bases are well known in the art and include amino acids such as arginine and lysine, mono-, di-, and triethanolamine, choline, mono-, di-, and trialkylamine, such as methylamine, dimethylamine, and trimethylamine, guanidine, N-benzylphenethylamine, N-methylglucosamine, N-methylpiperazine, morpholine, ethylendiamine, tris(hydroxymethyl)aminomethane and the like.

The invention also includes administration of hydrates of the compounds described herein, including, for example, solvates of the compounds described herein, pharmaceutical compositions comprising the solvates and methods of use of the solvates. In some embodiments, the invention is a solvate of a compound of Formula (I) or a pharmaceutical composition thereof.

Also included in the present invention are methods that include administering prodrugs of the compounds described herein, for example, prodrugs of a compound of Formula (I) or a pharmaceutical composition thereof or method of use of the prodrug.

The invention additionally includes use of clathrates of the compounds described herein, pharmaceutical compositions comprising the clathrates, and methods of use of the clathrates. In some embodiments, the invention is directed to clathrates of a compound of Formula (I) or a pharmaceutical composition thereof.

As discussed above, the invention includes administration of pharmaceutical compositions comprising a pharmaceutically acceptable carrier or excipient and a compound described herein. The compounds of Formula (I) or a pharmaceutically acceptable salt, solvate, clathrate or prodrug, can be administered in pharmaceutical compositions comprising a pharmaceutically acceptable carrier or excipient. The excipient can be chosen based on the expected route of administration of the composition in therapeutic applications. The route of administration of the composition depends on the condition to be treated. For example, intravenous injection may be preferred for treatment of a systemic disorder and oral administration may be preferred to treat a gastrointestinal disorder. The route of administration and the dosage of the composition to be administered can be determined by the skilled artisan without undue experimentation in conjunction with standard dose-response studies. Relevant circumstances to be considered in making those determinations include the condition or conditions to be treated, the choice of composition to be administered, the age, weight, and response of the individual patient, and the severity of the patient's symptoms. A pharmaceutical composition comprising a compound of Formula (I), or a pharmaceutically acceptable salt, solvate, clathrate or prodrug, can be administered by a variety of routes including, but not limited to, parenteral, oral, pulmonary, ophthalmic, nasal, rectal, vaginal, aural, topical, buccal, transdermal, intravenous, intramuscular, subcutaneous, intradermal, intraocular, intracerebral, intralymphatic, intraarticular, intrathecal and intraperitoneal. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the pharmacologic agent or composition. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like. Pharmaceutical compositions can also include large, slowly metabolized macromolecules such as proteins, polysaccharides such as chitosan, polylactic acids, polyglycolic acids and copolymers (such as latex functionalized SEPHAROSE™, agarose, cellulose, and the like), polymeric amino acids, amino acid copolymers, and lipid aggregates (such as oil droplets or liposomes).

The compositions can be administered parenterally such as, for example, by intravenous, intramuscular, intrathecal or subcutaneous injection. Parenteral administration can be accomplished by incorporating a composition into a solution or suspension. Such solutions or suspensions may also include sterile diluents such as water for injection, saline solution, fixed oils, polyethylene glycols, glycerine, propylene glycol or other synthetic solvents. Parenteral formulations may also include antibacterial agents such as, for example, benzyl alcohol or methyl parabens, antioxidants such as, for example, ascorbic acid or sodium bisulfate and chelating agents such as EDTA. Buffers such as acetates, citrates or phosphates and agents for the adjustment of tonicity such as sodium chloride or dextrose may also be added. The parenteral preparation can be enclosed in ampules, disposable syringes or multiple dose vials made of glass or plastic.

Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions.

Injectable formulations can be prepared either as liquid solutions or suspensions; solid forms suitable for solution in, or suspension in, liquid vehicles prior to injection can also be prepared. The preparation also can also be emulsified or encapsulated in liposomes or micro particles such as polylactide, polyglycolide, or copolymer for enhanced adjuvant effect, as discussed above [Langer, Science 249: 1527, 1990 and Hanes, Advanced Drug Delivery Reviews 28: 97-119, 1997]. The compositions and pharmacologic agents described herein can be administered in the form of a depot injection or implant preparation which can be formulated in such a manner as to permit a sustained or pulsatile release of the active ingredient.

Additional formulations suitable for other modes of administration include oral, intranasal, and pulmonary formulations, suppositories, transdermal applications and ocular delivery. For suppositories, binders and carriers include, for example, polyalkylene glycols or triglycerides; such suppositories can be formed from mixtures containing the active ingredient in the range of about 0.5% to about 10%, preferably about 1% to about 2%. Oral formulations include excipients, such as pharmaceutical grades of mannitol, lactose, starch, magnesium stearate, sodium saccharine, cellulose, and magnesium carbonate. Topical application can result in transdermal or intradermal delivery. Transdermal delivery can be achieved using a skin patch or using transferosomes. [Paul et al., Eur. J. Immunol. 25: 3521-24, 1995; Cevc et al., Biochem. Biophys. Acta 1368: 201-15, 1998].

For the purpose of oral therapeutic administration, the pharmaceutical compositions can be incorporated with excipients and used in the form of tablets, troches, capsules, elixirs, suspensions, syrups, wafers, chewing gums and the like. Tablets, pills, capsules, troches and the like may also contain binders, excipients, disintegrating agent, lubricants, glidants, sweetening agents, and flavoring agents. Some examples of binders include microcrystalline cellulose, gum tragacanth or gelatin. Examples of excipients include starch or lactose. Some examples of disintegrating agents include alginic acid, corn starch and the like. Examples of lubricants include magnesium stearate or potassium stearate. An example of a glidant is colloidal silicon dioxide. Some examples of sweetening agents include sucrose, saccharin and the like. Examples of flavoring agents include peppermint, methyl salicylate, orange flavoring and the like. Materials used in preparing these various compositions should be pharmaceutically pure and non-toxic in the amounts used. In another embodiment, the composition is administered as a tablet or a capsule.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor, and the like. For vaginal administration, a pharmaceutical composition may be presented as pessaries, tampons, creams, gels, pastes, foams or spray.

The pharmaceutical composition can also be administered by nasal administration. As used herein, nasally administering or nasal administration includes administering the composition to the mucus membranes of the nasal passage or nasal cavity of the patient. As used herein, pharmaceutical compositions for nasal administration of a composition include therapeutically effective amounts of the compounds prepared by well-known methods to be administered, for example, as a nasal spray, nasal drop, suspension, gel, ointment, cream or powder. Administration of the composition may also take place using a nasal tampon or nasal sponge.

For topical administration, suitable formulations may include biocompatible oil, wax, gel, powder, polymer, or other liquid or solid carriers. Such formulations may be administered by applying directly to affected tissues, for example, a liquid formulation to treat infection of conjunctival tissue can be administered dropwise to the subject's eye, or a cream formulation can be administered to the skin.

Rectal administration includes administering the pharmaceutical compositions into the rectum or large intestine. This can be accomplished using suppositories or enemas. Suppository formulations can easily be made by methods known in the art. For example, suppository formulations can be prepared by heating glycerin to about 120° C., dissolving the pharmaceutical composition in the glycerin, mixing the heated glycerin after which purified water may be added, and pouring the hot mixture into a suppository mold.

Transdermal administration includes percutaneous absorption of the composition through the skin. Transdermal formulations include patches, ointments, creams, gels, salves and the like.

In addition to the usual meaning of administering the formulations described herein to any part, tissue or organ whose primary function is gas exchange with the external environment, for purposes of the present invention, “pulmonary” will also mean to include a tissue or cavity that is contingent to the respiratory tract, in particular, the sinuses. For pulmonary administration, an aerosol formulation containing the active agent, a manual pump spray, nebulizer or pressurized metered-dose inhaler as well as dry powder formulations are contemplated. Suitable formulations of this type can also include other agents, such as antistatic agents, to maintain the disclosed compounds as effective aerosols.

A drug delivery device for delivering aerosols comprises a suitable aerosol canister with a metering valve containing a pharmaceutical aerosol formulation as described and an actuator housing adapted to hold the canister and allow for drug delivery. The canister in the drug delivery device has a head space representing greater than about 15% of the total volume of the canister. Often, the compound intended for pulmonary administration is dissolved, suspended or emulsified in a mixture of a solvent, surfactant and propellant. The mixture is maintained under pressure in a canister that has been sealed with a metering valve.

The invention also encompasses the treatment of a condition associated with a dysfunction in proteostasis in a subject comprising administering to said subject an effective amount of a compound of Formula (I) that enhances, improves or restores proteostasis of a protein. Proteostasis refers to protein homeostasis. Dysfunction in protein homeostasis is a result of protein misfolding, protein aggregation, defective protein trafficking or protein degradation. For example, the invention encompasses administering a compound of Formula (I) that corrects protein misfolding, reduces protein aggregation, corrects or restores protein trafficking and/or affects protein degradation for the treatment of a condition associated with a dysfunction in proteostasis. In some aspects of the invention, a compound of Formula (I) that corrects protein misfolding and/or corrects or restores protein trafficking is administered. In cystic fibrosis, the mutated or defective enzyme is the cystic fibrosis transmembrane conductance regulator (CFTR). One of the most common mutations of this protein is ΔF508 which is a deletion (Δ) of three nucleotides resulting in a loss of the amino acid phenylalanine (F) at the 508th (508) position on the protein. As described above, mutated cystic fibrosis transmembrane conductance regulator exists in a misfolded state and is characterized by altered trafficking as compared to the wild type CFTR. Additional exemplary proteins of which there can be a dysfunction in proteostasis, for example that can exist in a misfolded state, include, but are not limited to, glucocerebrosidase, hexosamine A, aspartylglucsaminidase, α-galactosidase A, cysteine transporter, acid ceremidase, acid α-L-fucosidase, protective protein, cathepsin A, acid β-glucosidase, acid β-galactosidase, iduronate 2-sulfatase, α-L-iduronidase, galactocerebrosidase, acid α-mannosidase, acid β-mannosidase, arylsulfatase B, arylsulfatase A, N-acetylgalactosamine-6-sulfate sulfatase, acid β-galactosidase, N-acetylglucosamine-1-phosphotransferase, acid sphingmyelinase, NPC-1, acid α-glucosidase, β-hexosamine B, heparin N-sulfatase, α-N-acetylglucosaminidase, α-glucosaminide N-acetyltransferase, N-acetylglucosamine-6-sulfate sulfatase, α-N-acetylgalactosaminidase, α-neuramidase, β-glucuronidase, β-hexosamine A and acid lipase, polyglutamine, α-synuclein, TDP-43, superoxide dismutase (SOD), Aβ peptide, tau protein transthyretin and insulin. The compounds of Formula (I) can be used to restore proteostasis (e.g., correct folding and/or alter trafficking) of the proteins described above.

Protein conformational diseases encompass gain of function disorders and loss of function disorders. In one embodiment, the protein conformational disease is a gain of function disorder. The terms “gain of function disorder,” “gain of function disease,” “gain of toxic function disorder” and “gain of toxic function disease” are used interchangeably herein. A gain of function disorder is a disease characterized by increased aggregation-associated proteotoxicity. In these diseases, aggregation exceeds clearance inside and/or outside of the cell. Gain of function diseases include, but are not limited to, neurodegenerative diseases associated with aggregation of polyglutamine, Lewy body diseases, amyotrophic lateral sclerosis, transthyretin-associated aggregation diseases, Alzheimer's disease, Machado-Joseph disease, cerebral B-amyloid angiopathy, retinal ganglion cell degeneration, tautopathies (progressive supranuclear palsy, corticobasal degeration, frontotemporal lobar degeneration), cerebral hemorrhage with amyloidosis, Alexander disease, Serpinopathies, familial amyloidotic neuropathy, senile systemic amyloidosis, ApoAI amyloidosis, ApoAII amyloidosis, ApoAIV amyloidosis, familial amyloidosis of the Finnish type, lysoyzme amyloidosis, fibrinogen amyloidosis, dialysis amyloidosis, inclusion body myositis/myopathy, cataracts, medullary thyroid carcinoma, cardiac atrial amyloidosis, pituitary prolactinoma, hereditary lattice corneal dystrophy, cutaneous lichen amyloidosis, corneal lactoferrin amyloidosis, corneal lactoferrin amyloidosis, pulmonary alveolar proteinosis, odontogenic tumor amyloid, seminal vesical amyloid, sickle cell disease, critical illness myopathy, von Hippel-Lindau disease, spinocerebellar ataxia 1, Angelman syndrome, giant axon neuropathy, inclusion body myopathy with Paget disease of bone, frontotemporal dementia (IBMPFD) and prion diseases. Neurodegenerative diseases associated with aggregation of polyglutamine include, but are not limited to, Huntington's disease, dentatorubral and pallidoluysian atrophy, several forms of spino-cerebellar ataxia, and spinal and bulbar muscular atrophy. Alzheimer's disease is characterized by the formation of two types of aggregates: extracellular aggregates of Aβ peptide and intracellular aggregates of the microtubule associated protein tau. Transthyretin-associated aggregation diseases include, for example, senile systemic amyloidoses and familial amyloidotic neuropathy. Lewy body diseases are characterized by an aggregation of α-synuclein protein and include, for example, Parkinson's disease, lewy body dementia (LBD) and multiple system atrophy (SMA). Prion diseases (also known as transmissible spongiform encephalopathies or TSEs) are characterized by aggregation of prion proteins. Exemplary human prion diseases are Creutzfeldt-Jakob Disease (CJD), Variant Creutzfeldt-Jakob Disease, Gerstmann-Straussler-Scheinker Syndrome, Fatal Familial Insomnia and Kuru. In another embodiment, the misfolded protein is alpha-1 anti-trypsin.

In a further embodiment, the protein conformation disease is a loss of function disorder. The terms “loss of function disease” and “loss of function disorder” are used interchangeably herein. Loss of function diseases are a group of diseases characterized by inefficient folding of a protein resulting in excessive degradation of the protein. Loss of function diseases include, for example, lysosomal storage diseases. Lysosomal storage diseases are a group of diseases characterized by a specific lysosomal enzyme deficiency which may occur in a variety of tissues, resulting in the build-up of molecules normally degraded by the deficient enzyme. The lysosomal enzyme deficiency can be in a lysosomal hydrolase or a protein involved in the lysosomal trafficking. Lysosomal storage diseases include, but are not limited to, aspartylglucosaminuria, Fabry's disease, Batten disease, Cystinosis, Farber, Fucosidosis, Galactasidosialidosis, Gaucher's disease (including Types 1, 2 and 3), Gml gangliosidosis, Hunter's disease, Hurler-Scheie's disease, Krabbe's disease, α-Mannosidosis, β-Mannosidosis, Maroteaux-Lamy's disease, Metachromatic Leukodystrophy, Morquio A syndrome, Morquio B syndrome, Mucolipidosis II, Mucolipidosis III, Neimann-Pick Disease (including Types A, B and C), Pompe's disease, Sandhoff disease, Sanfilippo syndrome (including Types A, B, C and D), Schindler disease, Schindler-Kanzaki disease, Sialidosis, Sly syndrome, Tay-Sach's disease and Wolman disease.

In another embodiment, the disease associated with a dysfunction in proteostasis is a cardiovascular disease. Cardiovascular diseases include, but are not limited to, coronary artery disease, myocardial infarction, stroke, restenosis and arteriosclerosis. Conditions associated with a dysfunction of proteostasis also include ischemic conditions, such as, ischemia/reperfusion injury, myocardial ischemia, stable angina, unstable angina, stroke, ischemic heart disease and cerebral ischemia.

In yet another embodiment, the disease associated with a dysfunction in proteostasis is diabetes and/or complications of diabetes, including, but not limited to, diabetic retinopathy, cardiomyopathy, neuropathy, nephropathy, and impaired wound healing.

In a further embodiment, the disease associated with a dysfunction in proteostasis is an ocular disease including, but not limited to, age-related macular degeneration (AMD), diabetic macular edema (DME), diabetic retinopathy, glaucoma, cataracts, retinitis pigmentosa (RP) and dry macular degeneration.

In yet additional embodiments, the method of the invention is directed to treating a disease associated with a dysfunction in proteostasis, wherein the disease affects the respiratory system or the pancreas. In certain additional embodiments, the methods of the invention encompass treating a condition selected from the group consisting of polyendocrinopathy/hyperinsulinemia, diabetes mellitus, Charcot-Marie Tooth syndrome, Pelizaeus-Merzbacher disease, and Gorham's Syndrome.

Additional conditions associated with a dysfunction of proteostasis include hemoglobinopathies, inflammatory diseases, intermediate filament diseases, drug-induced lung damage and hearing loss. The invention also encompasses methods for the treatment of hemoglobinopathies (such as sickle cell anemia), an inflammatory disease (such as inflammatory bowel disease, colitis, ankylosing spondylitis), intermediate filament diseases (such as non-alcoholic and alcoholic fatty liver disease) and drug induced lung damage (such as methotrexate-induced lung damage). The invention additionally encompasses methods for treating hearing loss, such as noise-induced hearing loss, aminoglycoside-induced hearing loss, and cisplatin-induced hearing loss.

Additional conditions include those associated with a defect in protein trafficking and that can be treated according to methods of the invention include: PGP mutations, hERG trafficking mutations, nephrongenic diabetes insipidus mutations in the arginine-vasopressin receptor 2, persistent hyperinsulinemic hypoglycemia of infancy (PHH1) mutations in the sulfonylurea receptor 1, and α1AT.

The invention is illustrated by the following examples which are not meant to be limiting in any way.

### EXEMPLIFICATION

### Example 1

### Preparation of Compounds 4, 13, 20, 41, and 329A

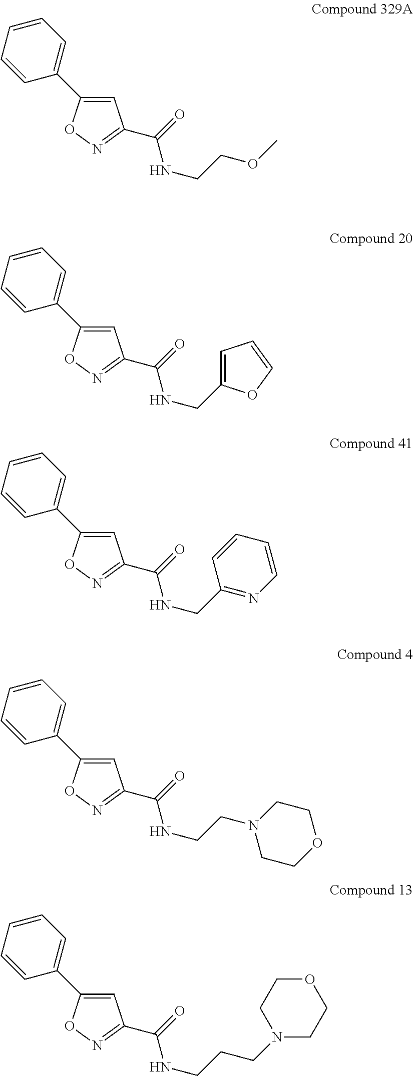 [SMILES: COCCNC(=O)c1cc(-c2ccccc2)on1 ; O=C(NCc1ccco1)c1cc(-c2ccccc2)on1 ; O=C(NCc1ccccn1)c1cc(-c2ccccc2)on1 ; O=C(NCCN1CCOCC1)c1cc(-c2ccccc2)on1 ; O=C(NCCCN1CCOCC1)c1cc(-c2ccccc2)on1]

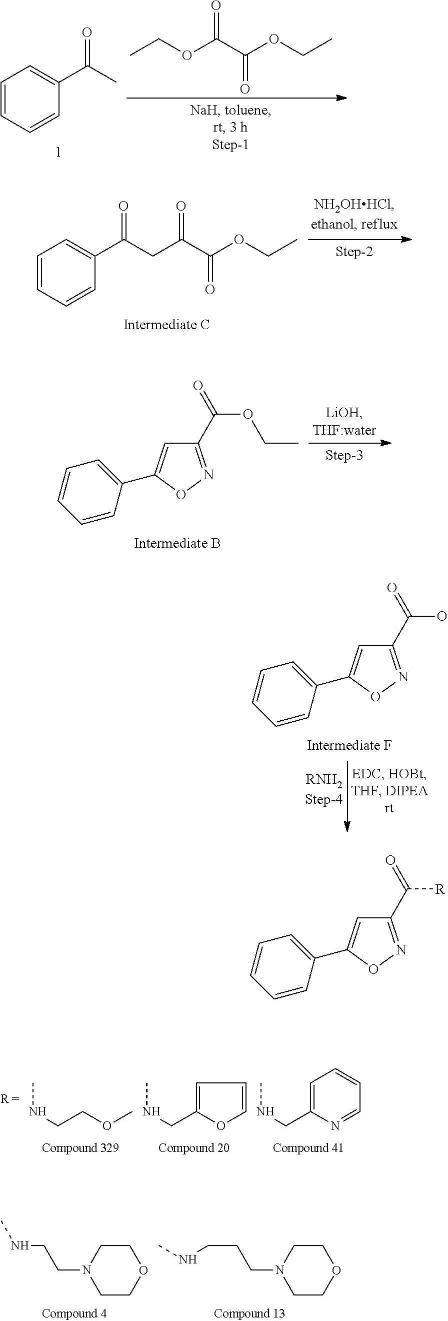

### i. Step 1: Synthesis of 4-(phenyl)-2,4-dioxo-butyric acid ethyl ester: Intermediate C

To a suspension of NaH (4.26 g, 0.107 mole) in dry toluene acetophenone (10 g, 0.083 mol) was added at room temperature (rt) and stirred for 60 min. After 60 min of stirring, a solution of diethyl oxalate (17 ml, 0.124 moles) in dry toluene was added drop wise and stirred at room temperature for 1 h. A sudden exotherm was observed, reaction mass turned dark brown. The progress of reaction was monitored by TLC. Reaction was worked up by evaporating toluene under vacuum. The resultant solid was diluted by ice water. Obtained solid was filtered to get desired compound. Compound was dried under vacuum. Yield—14 g (76.6%) of a yellow solid.

Analytical Data—¹H NMR (400 MHz, CDCl₃): δ 1.054-1.086 (t, 3H), 1.78-1.96 (bs, 2H), 3.88-3.89 (brs, 2H), 6.44 (s, 1H), 7.18-7.27 (m, 2H), 7.66-7.68 (d, 2H), LC-MS: (M+H)⁺=221.1 m/z. (97.24%).

### ii. Step-2: Synthesis of 5-(phenyl)-isoxazole-2-carboxylic acid ethyl ester: Intermediate B

To a solution of Intermediate C (14 g, 0.063 moles) in ethanol (100 ml), NH₂OH.HCl (5.7 g, 0.082 mole) was added and refluxed for 3 h. Progress of reaction was monitored by TLC. After completion, reaction mass was concentrated on rotary evaporator, diluted with water and extracted using EtOAc (3×100 mL). Organic layers were combined, dried over Na₂SO₄ and concentrated to dryness. Crude compound was purified by column chromatography using 100-200-mesh silica gel, and 10% EtOAc: Hexane. Intermediate B was isolated as low melting white solid. Yield—6.0 g (43.89%).

Analytical Data—¹H NMR (400 MHz, CDCl3) δ 1.41-1.45 (t, 2H), 4.41-4.43 (q, 2H), 6.91 (s, 1H), 7.45-7.49 (m, 3H), 7.78-7.81 (m, 2H). LC-MS: (M+H)⁺=218.1 m/z. (88%).

### iii. Step-3: Synthesis of 5-(phenyl)-isoxazole-2-carboxylic acid: Intermediate F

To a solution of Intermediate B (10.0 g, 0.046 mole) in THF: Water (100 ml), LiOH.H₂O (3.86 g, 0.0921 mole) was added at room temperature and stirred for 2 hrs. Progress of reaction was monitored by TLC. After completion, reaction mass was concentrated on rotary evaporator. Crude mass was diluted with water and acidified with dilute HCl. Resultant solid was filtered and dried under vacuum. Yield—7.1 g (82%).

Analytical Data—¹H NMR (400 MHz, CDCl3) δ 7.42 (s, 1H), 7.51-7.58 (m, 3H), 7.93-7.96 (m, 2H), 14.10 (bs, 1H). LC-MS: (M+H)⁺=190.1 m/z. (98.18%).

### iv. Step-4: Synthesis of 5-(phenyl)-isoxazole-2-carboxylic acid amide

To the solution of Intermediate F (0.4 g, 0.0021 mol) in THF, EDC.HCl (0.6 g, 0.0031 mol), and HOBT.H₂O (0.38 g, 0.0025 mol) was added at rt. Reaction was stirred at room temperature for one hr. Then amine (0.3 g, 0.0023 mol) and DIPEA (1.1 ml, 0.0063 mol) were added. Progress of reaction was monitored by TLC. After completion, the reaction was worked up by concentrating reaction mass on rotary evaporator. Crude solid was diluted by adding water. Aqueous was extracted by EtOAc (3×10 ml). Organic layer was dried over Na₂SO₄ and concentrated till dryness. Crude compound was purified by Combiflash to give the desired amide.

### v. Compounds 1, 13, 20, 41, and 329A were Prepared as Described Above

### Compound 329A

Yield: 0.250 g (48.07%)

Nature: Off White Solid

1H-NMR (400 MHz, CDCl3) δ: 3.38 (s, 3H), 3.54-3.57 (t, 2H), 3.63-3.67 (q, 2H), 6.95 (s, 1H), 7.17 (bS, 1H), 7.45-7.50 (m, 3H), 7.77-7.80 (m, 2H)

LCMS (M+H)⁺: 247.0 m/z

HPLC: 220 nm: 99.25%, 254 nm: 99.82%.

### Compound 20

Yield: 180 mg (32%)

Appearance: Off White Solid

Analytical Data—¹H NMR (400 MHz, CDCl3): δ 4.63-4.64 (d, 2H), 6.30-6.34 (m, 2H), 6.97 (s, 1H), 7.13 (bs, 1H), 7.381-7.83 (s, 1H), 7.46-7.49 (m, 3H), 7.77-7.79 (m, 2H)

LC-MS: (M+H)⁺=268.9 m/z. (99.29%)

HPLC: 220 nm: 97.63%, 254 nm: 99.16.

### Compound 41

Yield: 200 mg (34%)

Appearance: Off White Solid

Analytical Data—¹H NMR (400 MHz, CDCl3): δ 4.76-4.78 (s, 2H), 6.98 (s, 1H), 7.20-7.24 (m, 1H), 7.31-7.33 (broad d, 1H), 7.44-7.51 (m, 3H), 7.66-7.70 (m, 1H), 7.77-7.82 (m, 2H), 8.01 (bs, 1H), 8.58-8.59 (d, 1H)

LC-MS: (M+H)⁺=279.9 m/z. (99.30%)

HPLC: 220 nm: 98.8%, 254 nm: 99.32%.

### Compound 4

Yield: 0.410 g (65%)

Nature: Off White Solid

1H-NMR (400 MHz, CDCl3) δ: 2.50 (s, 4H), 2.58-2.61 (t, 2H), 3.54-3.58 (q, 2H), 3.72-3.74 (t, 4H), 6.95 (s, 1H), 7.33 (bs, 1H), 7.47-7.50 (m, 3H), 7.78-7.80 (dd, 2H)

LCMS (M+H)⁺: 301.9 m/z

HPLC: 220 nm: 98.43%, 254 nm: 99.69%.

### Compound 13

Yield: 0.290 g (43%)

Nature: Off White Solid

1H-NMR (400 MHz, CDCl3) δ: 1-75-1.80 (m, 2H), 2.50-2.56 (t, bs, 5H), 3.55-3.60 (q, 2H), 3.81-3.84 (t, 4H), 6.95 (s, 1H), 7.47-7.50 (m, 3H), 7.78-7.80 (dd, 2H), 8.66 (bs, 1H)

LCMS (M+H)⁺: 316.2 m/z

HPLC: 220 nm: 98.21%, 254 nm: 98.96%.

### Example 2

### Preparation of Compounds 186, 188, 195, 197, 198 and 298-303

| Com- | |
|---|---|
| pound | |
| No | STRUCTURE |
| Com- pound 186 | 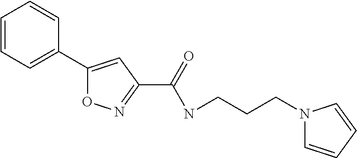 [SMILES: O=C(NCCCn1cccc1)c1cc(-c2ccccc2)on1] |
| Com- pound 198 | 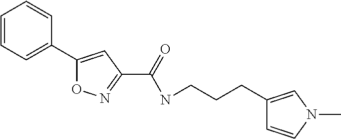 [SMILES: Cn1ccc(CCCNC(=O)c2cc(-c3ccccc3)on2)c1] |
| Com- pound 188 | 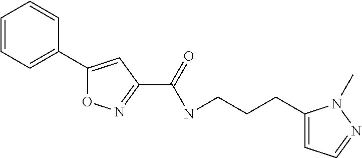 [SMILES: Cn1nccc1CCCNC(=O)c1cc(-c2ccccc2)on1] |
| Com- pound 195 | 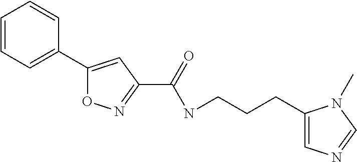 [SMILES: Cn1cncc1CCCNC(=O)c1cc(-c2ccccc2)on1] |
| Com- pound 197 | 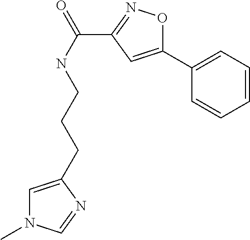 [SMILES: Cn1cnc(CCCNC(=O)c2cc(-c3ccccc3)on2)c1] |
| Com- pound 298 | 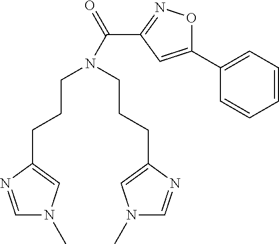 [SMILES: Cn1cnc(CCCN(CCCc2cn(C)cn2)C(=O)c2cc(-c3ccccc3)on2)c1] |
| Com- pound 299 | 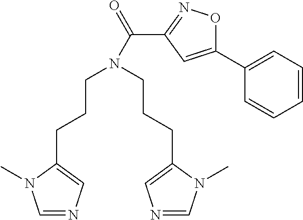 [SMILES: Cn1cncc1CCCN(CCCc1cncn1C)C(=O)c1cc(-c2ccccc2)on1] |
| Com- pound 189 | 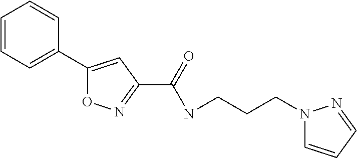 [SMILES: O=C(NCCCn1cccn1)c1cc(-c2ccccc2)on1] |
| Com- pound 204 | 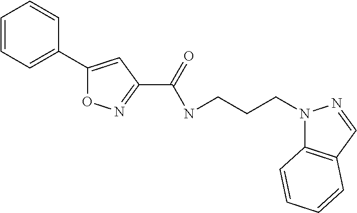 [SMILES: O=C(NCCCn1ncc2ccccc21)c1cc(-c2ccccc2)on1] |
| Com- pound 194 | 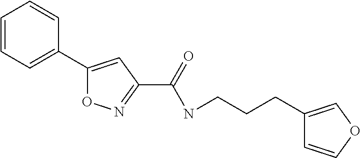 [SMILES: O=C(NCCCc1ccoc1)c1cc(-c2ccccc2)on1] |
| Com- pound 207 | 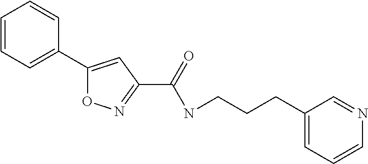 [SMILES: O=C(NCCCc1cccnc1)c1cc(-c2ccccc2)on1] |

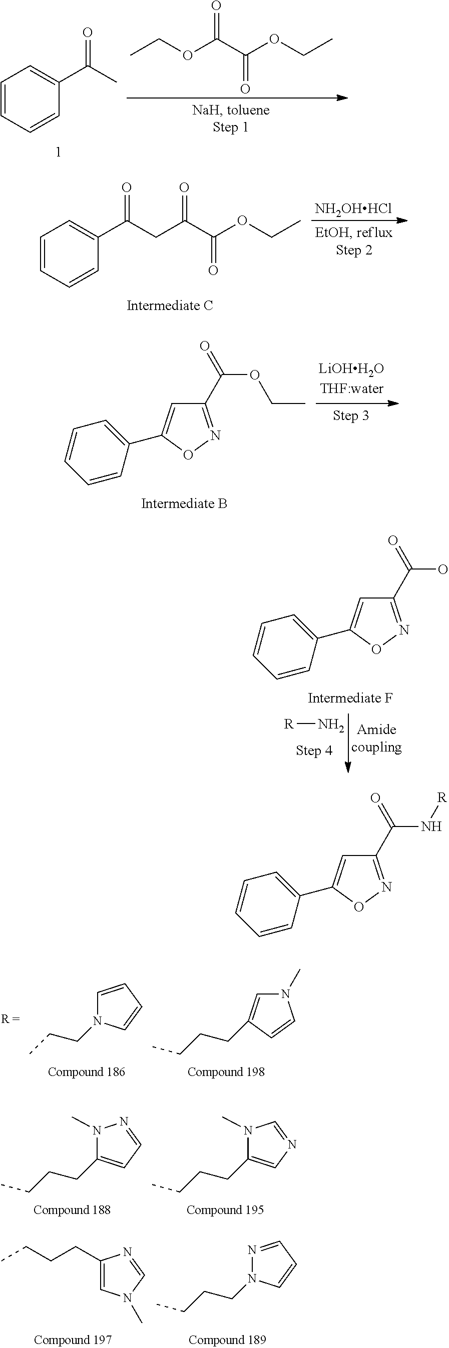

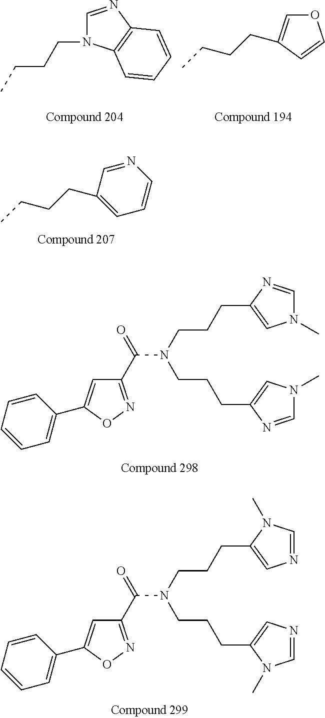

### i. Scheme A—Synthesis of Amine for Compound 186

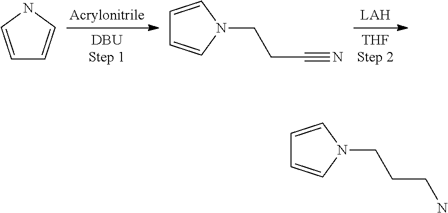 [SMILES: c1cc[nH]c1 ; N#CCCn1cccc1 ; NCCCn1cccc1]

The amine can be synthesized using methods described in the literature. For example, Step 1 in the scheme above can be performed as described in Murtagh et al. (2005), Novel amine-catalyzed hydroalkoxylation reactions of activated alkenes and alkynes, Chemical Communications 2: 227-229; Taylor et al. (2010), Friedel-Crafts Acylation of Pyrroles and Indoles using 1,5-Diazabicyclo[4.3.0]non-5-ene (DBN) as a Nucleophilic Catalyst Taylor, Organic Letters, 12(24), 5740-5743, Zhi et al. (2002) Synthesis of aminodihydro-1-pyrrolizinones, Journal of the Indian Chemical Society, 79(8), 698-700, the contents of each of which are expressly incorporated by reference herein. Step 2 in the scheme above can be performed as described in Senel et al. (2012), Development of a novel amperometric glucose biosensor based on copolymer of pyrrole-PAMAM dendrimers, Synthetic Metals, 162(7-8), 688-694; Merle et al. (2008), Electrode biomaterials based on immobilized laccase. Application for enzymatic reduction of dioxygen, Materials Science & Engineering, C: Biomimetic and Supramolecular Systems, 28(5-6), 932-938.

### ii. Scheme B—Synthesis of Amine for Compound 198

The final amine 3-(1-methylpyrrol-3-yl)propan-1-amine was prepared as shown in the scheme below.

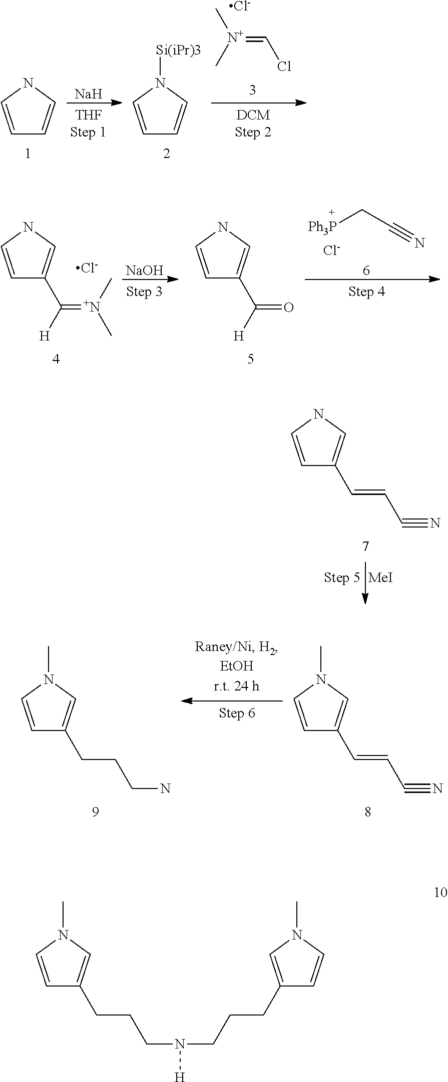

### Step-1: Synthesis of 1-Triisopropylsilanyl-1H-pyrrole (2)

To a stirred suspension of Sodium Hydride (2.68 g, 60% in oil, 0.1117 mol) in dry THF (50 mL) was added dropwise pyrrole (5.0 g) at 0° C. Reaction mixture was stirred at same temperature for 1.0 h. Then triisopropyl silyl chloride (18.67 g, 0.09688 mol) was added dropwise at 0° C. Resulting reaction mixture was then stirred at below 10° C. for 2 h. After completion of reaction, ice water was added (75 mL) and mixture was then extracted with diethyl ether (2×75 mL). Combined organic layer was then washed with water (100 mL). Organic layer was dried over sodium sulphate and evaporated under vacuum afforded red oily crude compound (15.5 g, 93.09% yield). This crude was forwarded as it is in next step.

### Step-2: Synthesis of (chloromethylene) dimethyl ammonium chloride (3)

In a 500 mL single neck RB flask was added N,N-dimethyl formamide (25.0 g, 342.0 mmol) under Nitrogen atmosphere and to this added freshly distilled thionyl chloride (40.69 g, 342.0 mmol) drop wise over a period of 15 min at rt. resulted reaction mixture was then warmed to 40° C. for 4 h. Slightly dense solution was observed. Excess solvent was evaporated under vacuum at 45° C. for 2 h to get white crystalline solid (35.0 g, 80% yield). This crude compound was directly carry forwarded to next step.

### Step-3 and Step-4: Synthesis of Isopropylidene-(1H-pyrrol-3-yl)-ammonium chloride (4) followed by 1H-Pyrrole-3-carbaldehyde (5)

To a stirred suspension of (chloromethylene) dimethyl ammonium chloride (3) (10.31 g, 80.57 mmol) in DCM (100 mL) was added 1-Triisopropylsilanyl-1H-pyrrole (2) (15.0 g, 67.14 mmol) in DCM (20 mL) at once at 0° C. under Nitrogen atmosphere. Resulted blackish reaction mixture was then refluxed at 45° C. for 30 min and cooled to 0° C. Precipitated solid was filtered and washed with diethyl ether (2×25 mL) to get intermediate 4 as brown solid. It was immediately dissolved in water (30 mL) and to this was added 2N NaOH solution (70 mL) at r.t. and stirred for 2 h at same temperature. After completion of reaction added ethyl acetate (100 mL) and stirred. Organic layer was separated and aqueous was again extracted with ethyl acetate (2×50 mL). Combined organic layer was washed with saturated brine solution (100 mL). Organic layer was dried over sodium sulphate and evaporated under vacuum afforded black solid compound 5 (2.4 g, 37.6%).

¹H NMR (400 MHz, DMSO) δ ppm=11.63 (bs, 1H), 9.69 (s, 1H), 7.62-7.64 (m, 1H), 6.90 (s, 1H), 6.45 (s, 1H), LCMS (M+H) 96.0.

### Step-5 Synthesis of 3-(1H-Pyrrol-3-yl)-acrylonitrile (7)

To a stirred solution of 1H-pyrrole-3-carbaldehyde (5) (2.2 g, 0.023 mol) in toluene (50 mL) was added Intermediate Wittig salt (6) (9.37 g, 0.027 mol) at r.t. To this resulted suspension was added DBU (4.57 g, 0.030 mol) drop wise at r.t. and heated to reflux at 115° C. for 1.5 h. After completion of reaction Toluene was distilled off completely under vacuum. Resulted crude oily mass was purified by silica gel column chromatography. Pure compound was eluted at 100% DCM. Evaporation of solvent afforded compound 7 (2.2 g, 80.5% yield) as off white solid.

### Step-6 Synthesis of 3-(1-Methyl-1H-pyrrol-3-yl)-acrylonitrile (8)

To a stirred solution of 3-(1H-Pyrrol-3-yl)-acrylonitrile (7) (2.2 g, 0.0186 mol) in DMF (25 mL) was added NaH (0.58 g, 60% in oil, 0.024 mol) lot wise at 0° C. Reaction mixture was stirred at same temperature for 5 min. To this was added Methyl iodide (3.17 g, 0.022 mol) at 0° C. dropwise. Resulted reaction mixture was stirred at 0° C. for 1 h. After completion of reaction ice water (75 mL) added. It was then extracted with ethyl acetate (3×30 mL). Combined organic layer was washed with water (3×30 mL). Organic layer was dried over sodium sulphate and evaporated completely under vacuum afforded oily residue. It was washed with pentane (10 mL). After drying afforded compound 8 (2.0 g, 81.30% yield) as yellow oil.

### Step-7 Synthesis of 3-(1-Methyl-1H-pyrrol-3-yl)-propylamine (9) and (10)

To a stirred solution of 3-(1-Methyl-1H-pyrrol-3-yl)-acrylonitrile (10) (1.0 g, 0.0075 mol) in Ethanol (20 mL) was added Raney Ni (0.5 g, 50% in water suspension) at r. t. Reaction mixture was then stirred under Hydrogen atmosphere for 18 h at r.t. After completion of reaction filtered it through celite and bed was washed with Methanol (30 mL). Filtrate was evaporated under vacuum. Crude obtained was purified through Neutral aluminum oxide column chromatography. Two spots were separated Spot-1 (10) was eluted with 5% Methanol in DCM and spot-2 (9) was eluted by adding 1% NH₄OH solution. Evaporation of spot-1 fraction gave compound 10 amine (0.25 g, 25%) as pale yellow liquid. While evaporation of spot-2 fraction gave compound 9 (0.53 g, 52%) as pale yellow liquid.

Analytical data (10): 1H NMR (400 MHz, CDCl₃) δ: 6.49-6.48 (t, 2H), 6.37 (s, 2H), 5.96-5.96 (t, 2H), 3.58 (6H, s), 2.68-2.64 (t, 4H), 2.48-2.45 (t, 4H), 1.80-2.10 (bs, 1H), 1.80-1.73 (m, 4H); LCMS (M+H) 260.3.

Analytical data (9): 1H NMR (400 MHz, CDCl₃) δ: 6.50-6.49 (t, 1H), 6.38 (1H, bs), 5.97-5.96 (t, 1H), 3.58 (3H, s), 2.74-2.71 (t, 2H), 2.50-2.45 (2H, t), 1.73-1.66 (m, 2H), 1.2-1.5 (2H, bs), LCMS (M+H) 139.0.

Steps 1, 2 and 3 can be performed as described in Arikawa et al. (2012). Discovery of a Novel Pyrrole Derivative 1-[5-(2-Fluorophenyl)-1-(pyridin-3-ylsulfonyl)-1H-pyrrol-3-yl]-N-methylmethanamine Fumarate (TAK-438) as a Potassium-Competitive Acid Blocker (P-CAB). Journal of Medicinal Chemistry 55(9), 4446-4456; Morrison et al. (2009), Synthesis of Pyrrolnitrin and Related Halogenated Phenylpyrroles, Organic Letters, 2009, 11(5), 1051-1054; Purkarthofer et al. (2005), Tetrahedron, 2005, 61(32), 7661-7668; Downie et al. (1993), Vilsmeier formylation and glyoxylation reactions of nucleophilic aromatic compounds using pyrophosphoryl chloride, Tetrahedron 49(19), 4015-34, the contents of each of which are expressly incorporated by reference herein.

Reagent 6 can be synthesized as described in Peters et al. (2013), A modular synthesis of teraryl-based α-helix mimetics, Part 1: Synthesis of core fragments with two electronically differentiated leaving groups, Chemistry—A European Journal, 19(7), 2442-2449; Aitken et al. (2006), Synthesis, thermal reactivity, and kinetics of stabilized phosphorus ylides. Part 2: [(Arylcarbamoyl)(cyano)methylene]triphenylphosphoranes and their thiocarbamoyl analogues, International Journal of Chemical Kinetics, 38(8), 496-502; Abramovitch et al. (1980), Ring contraction of 2-azidoquinoline and quinoxaline 1-oxides, Journal of Organic Chemistry 45(26), 5316-19; the contents of which are expressly incorporated by reference herein.

### iii. Scheme C-Synthesis of Amine for Compound 188

The amine 3-methyl-1H-pyrazol-5-yl)propan-1-amine was prepared as described in scheme C below.

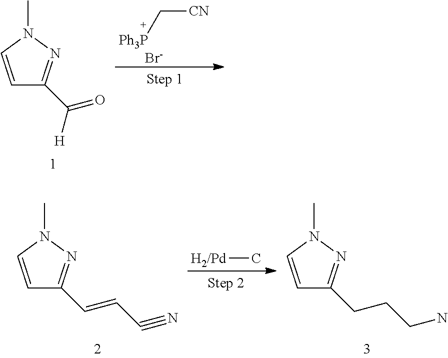

### Step-1: 3-(2-Methyl-2H-pyrazol-3-yl)-acrylonitrile (2)

To a stirred solution of 2-Methyl-2H-pyrazole-3-carbaldehyde (1.00 g, 0.0099 mol) in toluene (30 mL) was added Wittig salt (3.37 g, 0.0099 mol) at room temperature. To this resulted suspension was added DBU (1.52 mL, 0.0099 mole) drop wise and heated to reflux for 3 h. After completion of reaction toluene was distilled off completely under vacuum. Resulted crude oily mass was purified on combi flash. Pure Evaporation of solvent afforded compound 2 (0.450 g, 41.32% yield) as White Solid.

Analytical data 1H NMR (400 MHz, CDCl₃) δ: 3.93 (s, 3H), 5.75, 5.79 (s, s, 1H total), 6.56-6.57 (d, 1H), 7.26, 7.30 (s, s, 1H total), 7.46-7.47 (d, 1H).

### Step-2: 3-(2-Methyl-2H-pyrazol-3-yl)-propylamine (3)

To a stirred solution of 3-(2-Methyl-2H-pyrazol-3-yl)-acrylonitrile (0.450 g, 0.00338 mol) in ethanol (10 mL) was added Raney Ni (1 g, 50% in water suspension) at room temperature. Reaction mixture was then stirred under Hydrogen atmosphere for 18 h. After completion of reaction was filtered through celite bed and was washed with ethanol (5×2 mL). Filtrate was evaporated under vacuum. Crude obtained was purified through neutral aluminum oxide column chromatography. Pure compound was eluted at 10% Methanol in DCM and 1% Ammonia solution. Evaporation of solvent afforded Compound 3 (0.210 g, 46.77% yield) as brownish liquid.

Analytical data 1H NMR (400 MHz, CDCl₃) δ: 1.4-1.6 (bs, 2H), 1.73-1.81 (m, 4H), 2.61-2.68 (t, 2H), 2.75-2.78 (t, 2H), 6.00 (d, 1H), 7.35 (d, 1H).

The Wittig reagent can be purchased or synthesized as described in the following references: Kiddie et al. (2000), Microwave irradiation in organophosphorus chemistry. Part 2: Synthesis of phosphonium salts, Tetrahedron Letters, 41(9), 1339-1341; Suzanne et al. (2007), C—H Activation Reactions of Ruthenium N-Heterocyclic Carbene Complexes: Application in a Catalytic Tandem Reaction Involving C—C Bond Formation from Alcohols Burling, Journal of the American Chemical Society, 129(7), 1987-1995; Yuan et al. (2011), Rational Design of a Highly Reactive Ratiometric Fluorescent Probe for Cyanide, Organic Letters 13(14), 3730-3733; the contents of each of which are expressly incorporated by reference herein.

### iv. Scheme D-Synthesis of Amine for Compound 195, 197, 298, and 299

The desired amines were prepared as described below in Scheme D. References describing the final amine include Durant et al. (1985), The histamine H2-receptor agonist impromidine: synthesis and structure activity considerations, Journal of Medicinal Chemistry 28(10), 1414-22; Durant et al. (1973), (Aminoalkyl) imidazoles GB 1341375 A 19731219; the contents of each of which are expressly incorporated by reference herein.

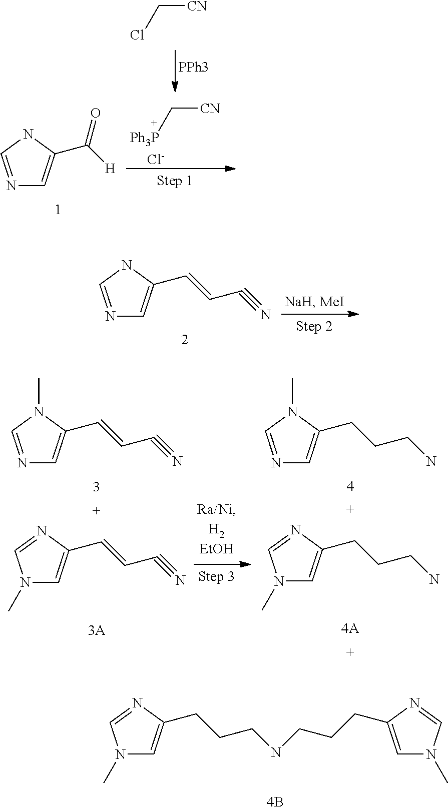

### Step-1: 3-(3H-Imidazol-4-yl)-acrylonitrile (2)

To a stirred solution of 3H-Imidazole-4-carbaldehyde (1) (1 g, 0.010 mole) in toluene (20 mL) was added Intermediate Wittig salt (A) (3.9 g, 0.011 mole) at room temperature. To this resulted suspension was added DBU (1.9 g, 0.013 mole) drop wise at room temperature and heated to reflux at 115° C. for 1.5 h. After completion of reaction, toluene was distilled off completely under vacuum. Resulted crude oily mass was purified by silica gel column chromatography (100-200 mesh). Pure compound was eluted at 100% DCM. Evaporation of solvent afforded compound 2 (1.0 g, 81%) as off white solid.

### Step-2: 3-(3-Methyl-3H-imidazol-4-yl)-acrylonitrile (3) and 3-(1-Methyl-1H-imidazol-4-yl)-acrylonitrile (3A)

To a stirred solution of 3-(3H-Imidazol-4-yl)-acrylonitrile (2) (2.5 g, 0.020 mol) in DMF (20 mL) was added NaH (0.65 g, 60% in oil, 0.027 mol) lot wise at 0° C. Reaction mixture was stirred at same temperature for 5 min. To this was added Methyl iodide (3.5 g, 0.025 mol) at 0° C. drop wise. Resulted reaction mixture was stirred at 0° C. for 1 h. After completion of reaction ice water (75 mL) added. It was then extracted with ethyl acetate (3×30 mL). Combined organic layer was washed with water (3×30 mL). Organic layer was dried over sodium sulphate and evaporated completely under vacuum afforded crude residue. Resulted crude oily mass was purified by flash column chromatography, eluted with 30% ethyl acetate in hexane gave spot 1 compound 3A (1.3 g 46%) and spot 2 compound 3 (0.1 g 3.5% yield).

Analytical Data 3

¹H NMR (400 MHz, CDCl₃) δ: 8.12 (s, 1H), 7.55-7.54 (d, 1H), 6.93-6.90 (d, 1H), 5.32-5.29 (d, 1H), 3.66 (s, 3H); LCMS [M+H] 134.1.

Analytical data (1H NMR) of compound 3A showed some extra peaks along with desired and the crude material was used directly as such for next step.

### Step-3: 3-(3-Methyl-3H-imidazol-4-yl)-polyamine (4)

To a stirred solution of 3-(3-Methyl-3H-imidazol-4-yl)-acrylonitrile (3) (0.24 g, 0.001 mol) in Ethanol (10 mL) was added Raney Ni (0.2 g, 50% in water suspension) at rt. Reaction mixture was then stirred under Hydrogen atmosphere for 18 h at r.t. After completion of reaction filtered it through celite and bed was washed with Methanol (20 mL). Filtrate was evaporated under vacuum. Crude obtained was purified through Neutral aluminum oxide column chromatography pure compound was eluted in 5% Methanol in DCM and 1% Ammonia solution gave (0.12 g 48% yield) of compound (4). Analytical data 1H NMR (400 MHz, CDCl₃) δ: 7.36 (s, 1H), 6.76-6.4 (1H, d), 3.54 (t, 3H), 2.80-2.76 (t, 2H), 2.59-2.55 (t, 2H), 1.80-1.73 (m, 2H), 1.18 (bs, 2H); LCMS [M+H] 140.1.

### Step-3: 3-(1-Methyl-1H-imidazol-4-yl)-polyamine (4A) and Bis-[3-(1-methyl-1H-imidazol-4-yl)-Propyl]-amine (4B)

To a stirred solution of 3-(1-Methyl-1H-imidazol-4-yl)-acrylonitrile (3A) (0.8 g, 0.006 mol) in Ethanol (20 mL) was added Raney Ni (0.5 g, 50% in water suspension) at r. t. Reaction mixture was then stirred under Hydrogen atmosphere for 18 h at r.t. After completion of reaction filtered it through celite and bed was washed with Methanol (30 mL). Filtrate was evaporated under vacuum. Crude obtained was purified through Neutral aluminum oxide column chromatography spot 1 was eluted at 5% Methanol in DCM gave 4B (0.35 g 42% yield) and spot 2 was eluted at 5% Methanol in DCM and 1% Ammonia solution gave 4A (0.27 g 32.5% yield).

Analytical Data (4B) Spot-1

1H NMR (400 MHz, CDCl₃) δ ppm=7.29 (s, 2H), 6.60 (s, 2H), 3.60 (s, 6H), 3.45 (s, 1H), 2.74-2.70 (t, 4H), 2.61-2.57 (t, 4H), 1.91-1.85 (m, 4H), 1.23 (s, 4H); LCMS [M+H] 262.3.

Analytical Data-CR928-116-108-04 (4A) Spot-2

¹H NMR (400 MHz, CDCl₃) δ: 7.30 (s, 1H), 6.58 (s, 1H), 2.73-2.70 (t, 2H), 2.59-2.55 (t, 2H), 1.80-1.72 (m, 2H), 1.4-1.6 (bs, 2H); LCMS [M+H] 140.

### v. Scheme E—Synthesis of Furanyl Amine for the Synthesis of Compound 194

The synthesis of 3-Furanpropanamine can be carried out as shown below. In addition it is available from commercial sources and described in two patent publications: U.S. Patent Application Publication No. 20040087601 (Preparation of pyrimidine amino acid derivatives as interleukin-8 (IL-8) receptor antagonists and WO 2004063192 (Preparation of imidazolyl pyrimidine derivatives for therapeutic use as interleukin 8 (IL-8) receptor modulators), the contents of which are expressly incorporated by reference herein.

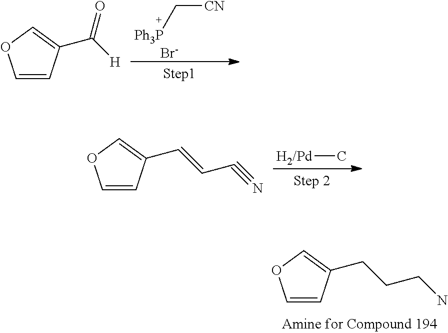

### Step-1: 3-Furan-3-yl-acrylonitrile

To a stirred solution of Furan-3-carbaldehyde (0.500 g, 0.0520 mol) in toluene (5 mL) was added Wittig salt (5) (1.86 g, 0.00515 mol) (Synthesized using refluxing of Chloroacetonitrile and Triphenyl phosphine in toluene) at room temperature. To this resulted suspension was added DBU (0.78 mL, 0.00520 mol) drop wise and heated to reflux for 3 h. After completion of reaction toluene was distilled off completely under vacuum. Resulted crude oily mass was purified on Combiflash to afforded compound 3-Furan-3-yl-acrylonitrile (0.300 g, 60.12%) as colorless oil.

### Step-2: 3-Furan-3-Yl-Propylamine (Amine for Compound 194)

To a stirred solution of 3-Furan-3-yl-acrylonitrile (0.300 g, 0.00252 mol) in ethanol (5 mL) was added Raney Ni (0.5 g, 50% in water suspension) at room temperature. Reaction mixture was then stirred under 1 Atm of Hydrogen for 18 h. After completion, reaction was filtered through celite bed and washed with ethanol (5×2 mL). Filtrate was evaporated under vacuum. Crude mass obtained was purified using neutral aluminum oxide column chromatography. Pure compound was eluted with 5% Methanol in DCM and 1% Ammonia solution. Evaporation of solvent afforded 3-Furan-3-yl-propylamine (0.070 g, 23.4%) as pale yellow liquid. 1H NMR (400 MHz, CDCl3) δ 7.33 (s, 1H), 7.20 (s, 1H), 6.26 (s, 1H), 2.73-2.70 (t, 2H), 2.47-2.43 (t, 2H), 1.73-1.66 (m, 2H); LCMS [M+H] 126.

### Example 3

### Preparation of Compounds 142, 169, 177, 185 and 321

| Compound | |
|---|---|
| No. | STRUCTURE |
| Compound 321 | 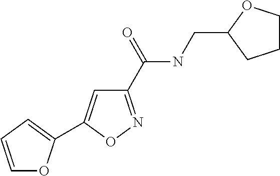 [SMILES: O=C(NCC1CCCO1)c1cc(-c2ccco2)on1] |
| Compound 169 | 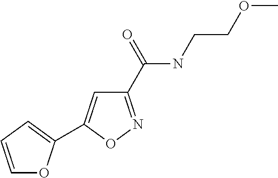 [SMILES: COCCNC(=O)c1cc(-c2ccco2)on1] |
| Compound 142 | 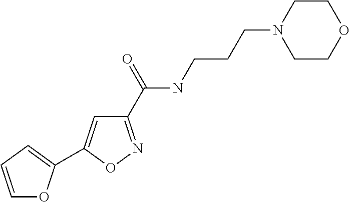 [SMILES: O=C(NCCCN1CCOCC1)c1cc(-c2ccco2)on1] |
| Compound 185 | 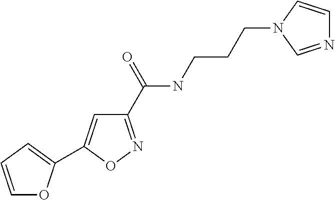 [SMILES: O=C(NCCCn1ccnc1)c1cc(-c2ccco2)on1] |
| Compound 177 | 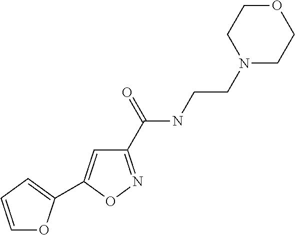 [SMILES: O=C(NCCN1CCOCC1)c1cc(-c2ccco2)on1] |

### i. Scheme for Synthesis of Compounds 142, 169, 185 and 321

The synthesis of the 2-furanyl derivatives shown below can be carried out using methods similar to those described for the phenyl derivative described above.

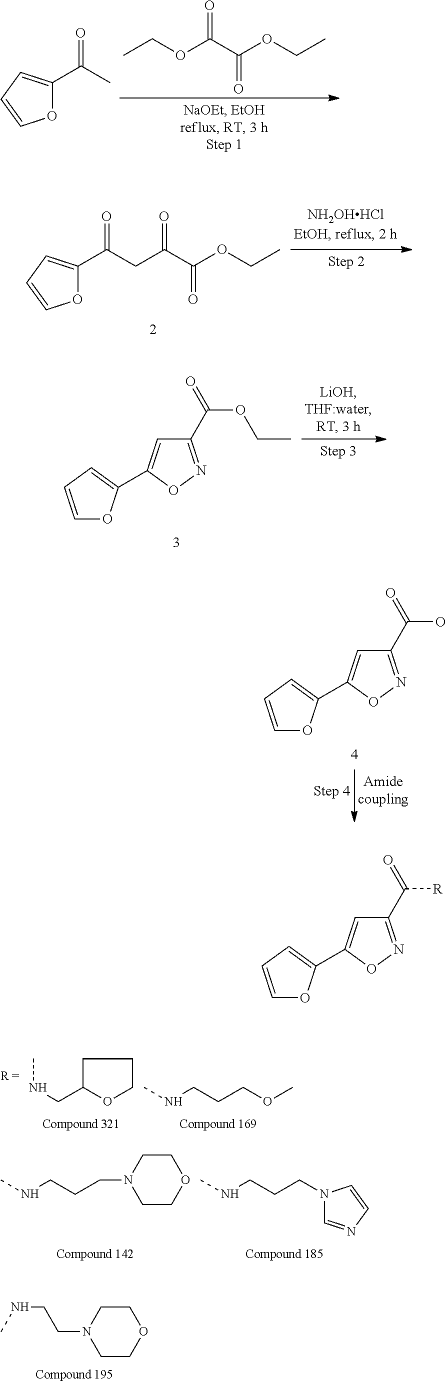

### ii. Scheme G for Synthesis of Amine for Compound 185

The amine for compound 185 was prepared as described below or the amine can be purchased from commercial vendors such as Aldrich. Synthesis of imidazole amine prepared as in BMCL, 18 (2008), 464-468: Carl P Bergstrom et al.

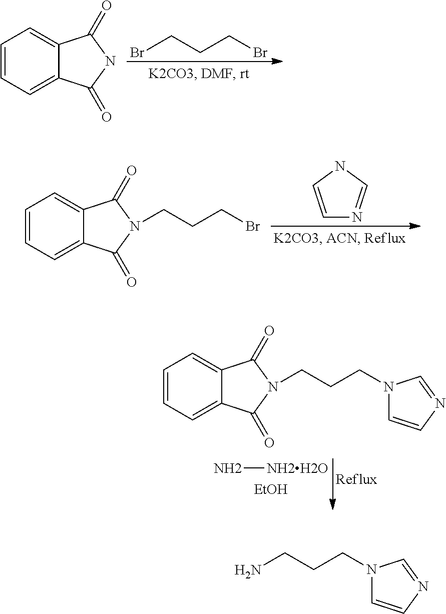

### Synthesis of 2-(3-Bromo-propyl)-isoindole-1,3-dione

To the solution of pthalamide (14.57 g, 0.1359 mol) in DMF (150 mL) was added K₂CO₃ (27.38 g, 0.2718 mol) at room temperature and stirred for 15 min. Then added 1,3 dibromopropane (20 g, 0.1359 mol) and stirred at room temperature for 2 h. Reaction was quenched with ice water and extracted using ethyl acetate. Organic layer was dried over Na₂SO₄, purified using 100-200 silica gel and eluted in 40% ethyl acetate-hexane. ¹H NMR (400 MHz, CDCl₃-d₆): δ 2.25 (q, 2H), 3.42 (t, 2H), 3.84 (t, 3H), 7.72 (dd, 2H), 7.85 (dd, 2H); LC-MS (M−H)⁻ 267.9.

### Synthesis of 2-(3-Imidazol-1-yl-propyl)-isoindole-1,3-dione

To the solution of compound 2-(3-Bromo-propyl)-isoindole-1,3-dione (6.8 g, 0.0253 mol) and Imidazole (3.4 g, 0.05072 mol) in Acetonitrile (50 mL) was added K₂CO₃ (7 g, 0.05072 mol) and reflux for 3 h. After completion of reaction, reaction was quenched with 50 mL water and extracted using ethyl acetate. Organic layer was dried over Na₂SO₄, purified over 100-200 silica gel and eluted in 10% MeOH:Dichloromethane (DCM) to obtain product 2-(3-Imidazol-1-yl-propyl)-isoindole-1,3-dione (3.5 g, 51%). ¹H NMR (400 MHz, CDCl₃): δ 2.18 (q, 2H), 3.73 (t, 2H), 4.00 (t, 2H), 6.98 (s, 1H), 7.03 (s, 1H), 7.55 (s, 1H), 7.73 (dd, 2H), 7.85 (dd, 2H); LC-MS (M+H)⁺ 256.0.

### Synthesis of 3-Imidazol-1-yl-propylamine

To the solution of compound 2-(3-Imidazol-1-yl-propyl)-isoindole-1,3-dione (3.5 g, 0.02796 mol) in ethanol was added Hydrazine hydrate (2.7 g, 0.05592 mol) and refluxed for 4 h. After completion of the reaction, solid was filtered and washed with ethanol, filtrate was concentrated, purified over neutral alumina and eluted in 5% MeOH: DCM to afford the product 4 (0.6 g). ¹H NMR (400 MHz, CDCl₃): δ 1.88 (m, 2H), 2.70 (t, 2H), 4.03 (t, 2H), 6.90 (s, 1H), 7.04 (s, 1H), 7.46 (s, 1H).

### Example 4

### CFTR Activity Assays

i. Using Measurements

Primary lung epithelial cells (hBEs) homozygous for the Cystic Fibrosis-causing ΔF508 mutation were differentiated for a minimum of 4 weeks in an air-liquid interface on SnapWell filter plates prior to the Ussing measurements. Cells were apically mucus-washed for 30 minutes prior to treatment with compounds. The basolateral media was removed and replaced with media containing the compound of interest diluted to its final concentration from DMSO stocks. Treated cells were incubated at 37° C. and 5% CO₂ for 24 hours. At the end of the treatment period, the cells on filters were transferred to the Ussing chamber and equilibrated for 30 minutes. The short-circuit current was measured in voltage clamp-mode (V_{hold}=0 mV), and the entire assay was conducted at a temperature of 36° C.-36.5° C. Once the voltages stabilized, the chambers were clamped, and data was recorded by pulse readings every 5 seconds. Following baseline current stabilization, the following additions were applied and the changes in current and resistance of the cells monitored:
1. Benzamil to the apical chamber to inhibit ENaC sodium channel.
2. Forskolin to both chambers to activate ΔF508-CFTR by phosphorylation.
3. Genistein to both chambers to potentiate ΔF508-CFTR channel opening.
4. CFTRinh-172 to the apical chamber to inhibit ΔF508-CFTR Cl− conductance.

The inhibitable current (that current that is blocked by CFTRinh-172) was measured as the specific activity of the ΔF508-CFTR channel, and increases in response to compound in this activity over that observed in vehicle-treated samples were identified as the correction of ΔF508-CFTR function imparted by the compound tested. ++ indicates activity ≧25% of VX-809 (1 uM) with compound at 10 uM and VX-809 at 1 uM; ** indicates activity ≧200% of VX-809 (1 uM) with compound at 10 uM and VX-809 at 1 uM; ** indicates activity 100-200% of VX-809 (1 uM) with compound at 10 uM and VX-809 at 1 uM; The transepithelial resistance (TER) for these compounds are within 30% of DMSO controls.

Using Activity

| | Solo | Combination |
|---|---|---|
| Compound | % VX809 | % VX809 |
| 16 | ++ | ** |
| 18 | ++ | ** |
| 9 | ++ | * |

### ii. hBE Equivalent Current (Ieq) Assay

Primary lung epithelial cells homozygous for the Cystic Fibrosis-causing ΔF508 mutation were differentiated for a minimum of 4 weeks in an air-liquid interface on Costar 24 well HTS filter plates prior to the equivalent current (Ieq) measurements. Cells were apically mucus-washed for 30 minutes 24 h prior to treatment with compounds. The basolateral media was removed and replaced with media containing the compound of interest diluted to its final concentration from DMSO stocks. Treated cells were incubated at 37° C. and 5% CO₂ for 24 hours. At the end of the treatment period, the media was changed to the Ieq experimental solution for 30 minutes before the experiment and plates are maintained in a CO₂-free incubator during this period. The plates containing the cells were then placed in pre-warmed heating blocks at 36° C.±0.5 for 15 minutes before measurements are taken. The transepithelial voltage (V_{T}) and conductance (G_{T}) were measured using a custom 24 channel current clamp (TECC-24) with 24 well electrode manifold. The Ieq assay measurements were made following additions with standardized time periods:
1. The baseline V_{T} and G_{T} values were measured for approximately 20 minutes.
2. Benzamil was added to block ENaC for 15 minutes.
3. Forskolin plus VX-770 were added to maximally activate ΔF508-CFTR for 27 minutes.
4. Bumetanide was added to inhibit the NaK₂Cl cotransporter and shut-off secretion of chloride.

The activity data captured was the area under the curve (AUC) for the traces of the equivalent chloride current. The AUC was collected from the time of the forskolin/VX-770 addition until the inhibition by bumetanide addition. Correction in response to compound treatment was scored as the increase in the AUC for compound-treated samples over that of vehicle-treated samples. (++ indicates activity ≧25% run at 10 uM of VX-809 at 1 uM, + indicates activity 10 to ≦25% run at 10 uM of VX-809 at 1 uM.

| Compound | I_{eq} |
|---|---|
| Number | hBE Activity |
| 237 | ++ |
| 16 | ++ |
| 110 | ++ |
| 223 | ++ |
| 197 | ++ |
| 13 | ++ |
| 329B | ++ |
| 233 | + |
| 330 | ++ |
| 18 | ++ |
| 214 | ++ |
| 8 | ++ |
| 212 | ++ |
| 19 | ++ |
| 92 | ++ |
| 228 | ++ |
| 120 | ++ |
| 207 | ++ |
| 6 | ++ |
| 217 | ++ |
| 188 | ++ |
| 5 | ++ |
| 115 | ++ |
| 204 | ++ |
| 2 | ++ |
| 153 | ++ |
| 14 | ++ |
| 225 | ++ |
| 4 | ++ |
| 198 | ++ |
| 90 | ++ |
| 186 | ++ |
| 35 | ++ |
| 1 | ++ |
| 336 | ++ |
| 65 | ++ |
| 36 | ++ |
| 234 | ++ |
| 335 | ++ |
| 8 | ++ |
| 329A | ++ |
| 342 | ++ |
| 226 | ++ |
| 7 | ++ |
| 292 | ++ |
| 11 | ++ |
| 195 | ++ |
| 101 | ++ |
| 201 | ++ |
| 114 | ++ |
| 70 | ++ |
| 102 | ++ |
| 12 | ++ |
| 232 | ++ |
| 95 | ++ |
| 120 | ++ |
| 230 | ++ |
| 349 | ++ |
| 191 | ++ |
| 200 | ++ |
| 52 | ++ |
| 238 | ++ |
| 332 | ++ |
| 144 | ++ |
| 205 | ++ |
| 192 | ++ |
| 97 | ++ |
| 224 | ++ |
| 373 | ++ |
| 376 | ++ |
| 377 | ++ |
| 378 | ++ |
| 372 | ++ |
| 218 | ++ |
| 189 | ++ |
| 270 | + |
| 51 | + |
| 135 | + |
| 295 | + |
| 286 | + |
| 150 | + |
| 15 | + |
| 221 | + |
| 10 | + |
| 30 | + |
| 276 | + |
| 17 | + |
| 343 | + |
| 41 | + |
| 375 | + |
| 229 | + |
| 338 | + |
| 94 | + |
| 135 | + |
| 220 | + |
| 321 | + |
| 71 | + |
| 194 | + |
| 238 | + |
| 100 | + |
| 64 | + |
| 374 | + |
| 326 | + |
| 172 | + |
| 344 | + |
| 128 | + |
| 27 | + |
| 283 | + |
| 20 | + |
| 161 | + |
| 345 | + |
| 256 | + |
| 239 | + |

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of modulating cystic fibrosis transmembrane conductance regulator (CFTR) activity in a subject in need thereof comprising administering to said subject an effective amount of a compound having the Formula (I): 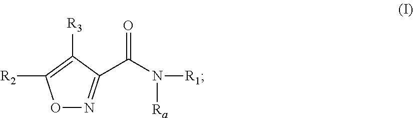
or a pharmaceutically acceptable salt, prodrug or solvate thereof, wherein:
R₁ is selected from the group consisting of: 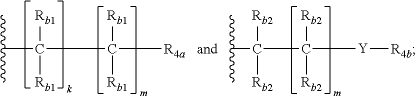
R₂ is selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted aryl, halo, OR_{c}, NR_{d}R_{d}, C(O)OR_{c}, NO₂, CN, C(O)R_{c}, C(O)C(O)R_{c}, C(O)NR_{d}R_{d}, NR_{d}C(O)R_{c}, NR_{d}S(O)ₙR_{c}, N(R_{d})(COOR_{c}), NR_{d}C(O)C(O)R_{c}, NR_{d}C(O)NR_{d}R_{d}, NR_{d}S(O)ₙNR_{d}R_{d}, NR_{d}S(O)ₙR_{c}, S(O)ₙR_{c}, S(O)ₙNR_{d}R_{d}, OC(O)OR_{c}, (C═NR_{d})R_{c}, optionally substituted heterocyclic and optionally substituted heteroaryl;
R₃ is selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted aryl, halo, OR_{c}, NR_{d}R_{d}, C(O)OR_{c}, NO₂, CN, C(O)R_{c}, C(O)C(O)R_{c}, C(O)NR_{d}R_{d}, NR_{d}C(O)R_{c}, NR_{d}S(O)ₙR_{c}, N(R_{d})(COOR_{c}), NR_{d}C(O)C(O)R_{c}, NR_{d}C(O)NR_{d}R_{d}, NR_{d}S(O)ₙNR_{d}R_{d}, NR_{d}S(O)ₙR_{c}, S(O)ₙR_{c}, S(O)ₙNR_{d}R_{d}, OC(O)OR_{c}, (C═NR_{d})R_{c}, optionally substituted heterocyclic and optionally substituted heteroaryl;
or alternatively, R₂ and R₃ can be taken together with the carbon atoms to which they are attached to form a fused, optionally substituted 3 to 12 membered cyclic group selected from the group consisting of optionally substituted C₃-C₁₂ cycloalkenyl, optionally substituted heterocyclic, optionally substituted aryl and optionally substituted heteroaryl;
R₄ₐ is selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted aryl, halo, OR_{c}, S(O)ₙR_{c}, NR_{d}R_{d}, C(O)OR_{c}, NO₂, CN, C(O)R_{c}, C(O)C(O)R_{c}, C(O)NR_{d}R_{d}, NR_{d}C(O)R_{c}, NR_{d}S(O)R_{c}, N(R_{d})(COOR_{c}), NR_{d}C(O)C(O)R_{c}, NR_{d}C(O)NR_{d}R_{d}, NR_{d}S(O)ₙR_{d}R_{d}, NR_{d}S(O)ₙR_{c}, S(O)NR_{d}R_{d}, OC(O)OR_{c}, (C═NR_{d})R_{c}, optionally substituted heterocyclic and optionally substituted heteroaryl;
R_{4b} is selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted aryl, optionally substituted heterocyclic and optionally substituted heteroaryl;
Rₐ is selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, C(O)OR_{c}, C(O)R_{c}, C(O)C(O)R_{c} and S(O)ₙR_{c};
or alternatively, Rₐ and the nitrogen atom to which it is attached is taken together with an adjacent C(R_{b1})(R_{b1}) or C(R_{b2})(R_{b2}) to form an optionally substituted, 4- to 12-membered heterocyclic ring containing one or more ring nitrogen atoms, wherein said heterocyclic ring optionally contains one or more ring heteroatoms selected from oxygen and sulfur;
each R_{b1} and R_{b2} is independently selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, halo, OR_{c}, NR_{d}R_{d}, C(O)OR_{c}, NO₂, CN, C(O)R_{c}, C(O)C(O)R_{c}, C(O)NR_{d}R_{d}, NR_{d}C(O)R_{c}, NR_{d}S(O)ₙR_{c}, N(R_{d})(COOR_{c}), NR_{d}C(O)C(O)R_{c}, NR_{d}C(O)NR_{d}R_{d}, NR_{d}S(O)ₙNR_{d}R_{d}, NR_{d}S(O)ₙR_{c}, S(O)ₙR_{c}, S(O)ₙNR_{d}R_{d}, OC(O)OR_{c} and (C═NR_{d})R_{c}; or alternatively, two geminal R_{b1} groups or two geminal R_{b2} groups and the carbon to which they are attached are taken together to form a C(O) group, or yet alternatively, two geminal R_{b1} groups or two geminal R_{b2} groups are taken together with the carbon atom to which they are attached to form a spiro C₃-C₁₂ cycloalkyl, a spiro C₃-C₁₂ cycloalkenyl, a spiro heterocyclic, a spiro aryl or spiro heteroaryl, each optionally substituted;
each R_{c} is independently selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl and optionally substituted heteroaryl;
Y is selected from the group consisting of S(O)ₙ, NRₙ, NR_{d}S(O)ₙ, NR_{d}S(O)ₙNR_{d}, NR_{d}C(O), NR_{d}C(O)O, NR_{d}C(O)C(O), NR_{d}C(O)NR_{d}, S(O)ₙNR_{d}, and O;
each R_{d} is independently selected from the group consisting of hydrogen, optionally substituted C₁-C₁₀ alkyl, optionally substituted C₂-C₁₀ alkenyl, optionally substituted C₂-C₁₀ alkynyl, optionally substituted C₁-C₁₀ alkoxy, optionally substituted C₃-C₁₂ cycloalkyl, optionally substituted C₃-C₁₂ cycloalkenyl, optionally substituted heterocyclic, optionally substituted aryl and optionally substituted heteroaryl; or two geminal R_{d} groups are taken together with the nitrogen atom to which they are attached to form an optionally substituted heterocyclic or an optionally substituted heteroaryl;
k is 0 or 1;
m is 0, 1, 2, 3, 4, or 5;
each n is independently 0, 1 or 2.

2. The method of claim 1, wherein R₁ is:

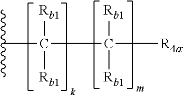

3. The method of claim 2, wherein R₁ is:

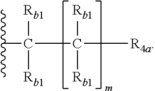

4. The method of claim 1, wherein R₁ is:

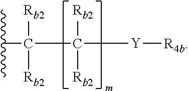

5. The method of claim 1, wherein m is 0, 1 or 2.

6. The method of claim 5, wherein m is 0.

7. The method of claim 5, wherein m is 1.

8. The method of claim 5, wherein m is 2.

9. The method of claim 4, wherein m is 1.

10. The method of claim 4, wherein Y is S(O)ₙ, P or NR_{d}.

11. The method of claim 1, wherein R₃ is hydrogen.

12. The method of claim 1, wherein R₃ is hydrogen or optionally substituted C₁-C₄ alkyl.

13. The method of claim 12, wherein Rₐ is hydrogen.

14. The method of claim 1, wherein each of R_{b1} and R_{b2} is independently selected from hydrogen, ORₑ, and optionally substituted C₁-C₁₀ alkyl, wherein Rₑ is hydrogen or optionally substituted C₁-C₁₀ alkyl.

15. The method of claim 1, wherein R₂ is selected from the group consisting of optionally substituted C₁-C₁₀ alkyl, optionally substituted C₃-C₁₂ cycloalkyl, optionally substituted C₃-C₁₂ cycloalkenyl, optionally substituted aryl, optionally substituted heterocyclic and optionally substituted heteroaryl.

16. The method of claim 15, wherein R₂ is selected from the group consisting of optionally substituted C₃-C₁₂ cycloalkyl, optionally substituted C₃-C₁₂ cycloalkenyl, optionally substituted aryl, optionally substituted heterocyclic and optionally substituted heteroaryl.

17. The method of claim 16, wherein R₂ is optionally substituted aryl.

18. The method of claim 17, wherein R₂ is optionally substituted phenyl.

19. The method of claim 17, wherein R₂ is unsubstituted phenyl.

20. The method of claim 18, wherein R₂ is a para-substituted phenyl.

21. The method of claim 16, wherein R₂ is optionally substituted heteroaryl.

22. The method of claim 21, wherein R₂ is optionally substituted thienyl or optionally substituted furanyl.

23. The method of claim 22, wherein R₂ is optionally substituted 2-thienyl.

24. The method of claim 21, wherein R₂ is optionally substituted pyridinyl.

25. The method of claim 1, wherein R₄ₐ is optionally substituted C₁-C₁₀ alkyl, optionally substituted C₃-C₁₂ cycloalkyl, optionally substituted C₃-C₁₂ cycloalkenyl, optionally substituted aryl, OR_{c}, C(O)OR_{c}, C(O)R_{c}, C(O)C(O)R_{c}, C(O)NR_{d}R_{d}, optionally substituted heterocyclic and optionally substituted heteroaryl.

26. The method of claim 25, wherein R₄ₐ is an optionally substituted heterocyclic or optionally substituted heteroaryl.

27. The method of claim 26, wherein R₄ₐ is cyclopentyl, tetrahydropyranyl, thiadiazolyl, oxazolidinonyl, tetrahydrofuranyl, oxazolinyl or morpholinyl, each optionally substituted.

28. The method of claim 27, wherein R₄ₐ is optionally substituted 2-tetrahydrofuranyl.

29. The method of claim 27, wherein R₄ₐ is optionally substituted N-morpholinyl.

30. The method of claim 26, wherein R₄ₐ is optionally substituted heteroaryl.

31. The method of claim 30, wherein R₄ₐ is optionally substituted heteroaryl containing one or more ring nitrogen atoms.

32. The method of claim 30, wherein R₄ₐ is selected from the group consisting of furanyl, pyridinyl, pyrazinyl, pyrazolyl, imidazolyl, isoxazolyl, triazolyl, thiazolyl, oxadiazolyl, thienyl, piperazinyl, and benzimidazolyl, each optionally substituted.

33. The method of claim 32, wherein R₄ₐ is optionally substituted 2-furanyl.

34. The method of claim 32, wherein R₄ₐ is optionally substituted N-methyl piperazinyl.

35. The method of claim 25, wherein R₄ₐ is ORₑ or C(O)NR_{d}R_{d}, wherein Rₑ is hydrogen or optionally substituted C₁-C₁₀ alkyl.

36. The method of claim 35, wherein R₄ₐ is C(O)NR_{d}R_{d}.

37. The method of claim 10, wherein Y is S, S(O)₂ or S(O)₂NR_{d}.

38. The method of claim 10, wherein Y is O.

39. The method of claim 10, wherein Y is NRₙ.

40. The method of claim 37, wherein R_{4b} is selected from the group consisting of hydrogen, optionally substituted C₁-C₁₀ alkyl, optionally substituted C₃-C₁₂ cycloalkyl, optionally substituted C₃-C₁₂ cycloalkenyl, optionally substituted aryl, optionally substituted heteroaryl and optionally substituted heterocyclic.

41. The method of claim 4, wherein R_{4b} is optionally substituted C₁-C₁₀ alkyl, optionally substituted C₃-C₁₂ cycloalkyl, optionally substituted C₃-C₁₂ cycloalkenyl, optionally substituted aryl, optionally substituted heterocyclic and optionally substituted heteroaryl.

42. The method of claim 41, wherein R_{4b} is an optionally substituted heterocyclic or optionally substituted heteroaryl.

43. The method of claim 42, wherein R_{4b} is tetrahydropyranyl, triazolyl, thiadiazolyl, tetrahydrofuranyl, or oxazolidinyl, each optionally substituted.

44. The method of claim 43, wherein R_{4b} is optionally substituted 2-tetrahydrofuranyl.

45. The method of claim 42, wherein is R_{4b} is an optionally substituted heteroaryl.

46. The method of claim 45, wherein R_{4b} is selected from the group consisting of furanyl, pyridinyl, pyrazinyl, pyrazolyl, imidazolyl, isoxazolyl, triazolyl, thiazolyl, oxadiazolyl, thienyl, and benzimidazolyl, each optionally substituted.

47. The method of claim 46, wherein R_{4b} is furanyl or imidazolyl, each optionally substituted.

48. The method of claim 18, wherein R₄ₐ is an optionally substituted heterocyclic or optionally substituted heteroaryl.

49. The method of claim 48, wherein R₃ is hydrogen.

50. The method of claim 49, wherein Rₐ is hydrogen or optionally substituted C₁-C₄ alkyl.

51. The method of claim 50, wherein Rₐ is hydrogen.

52. The method of claim 50, wherein each R_{b1} is independently selected from hydrogen, ORₑ, and optionally substituted C₁-C₁₀ alkyl, wherein Rₑ is optionally substituted C₁-C₁₀ alkyl.

53. The method of claim 1, wherein the compound is selected from the following Table:
TABLE 1B

| Com- | |
|---|---|
| pound | |
| No. | Chemical Structure |
| 1 | 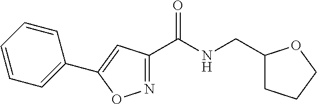 [SMILES: O=C(NCC1CCCO1)c1cc(-c2ccccc2)on1] |
| 2 | 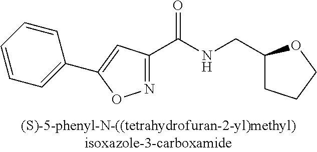 [SMILES: O=C(NC[C@@H]1CCCO1)c1cc(-c2ccccc2)on1] |
| 3 | 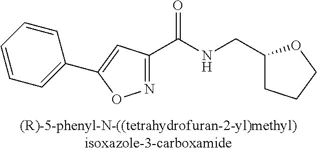 [SMILES: O=C(NC[C@H]1CCCO1)c1cc(-c2ccccc2)on1] |
| 4 | 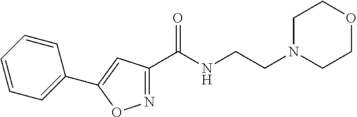 [SMILES: O=C(NCCN1CCOCC1)c1cc(-c2ccccc2)on1] |
| 5 | 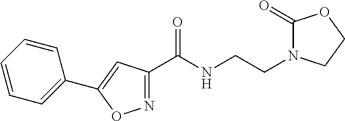 [SMILES: O=C(NCCN1CCOC1=O)c1cc(-c2ccccc2)on1] |
| 6 | 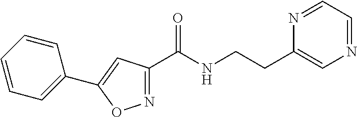 [SMILES: O=C(NCCc1cnccn1)c1cc(-c2ccccc2)on1] |
| 7 | 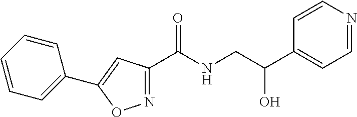 [SMILES: O=C(NCC(O)c1ccncc1)c1cc(-c2ccccc2)on1] |
| 8 | 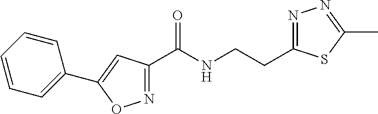 [SMILES: Cc1nnc(CCNC(=O)c2cc(-c3ccccc3)on2)s1] |
| 9 | 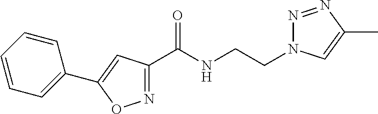 [SMILES: Cc1cn(CCNC(=O)c2cc(-c3ccccc3)on2)nn1] |
| 10 | 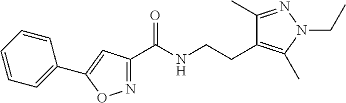 [SMILES: CCn1nc(C)c(CCNC(=O)c2cc(-c3ccccc3)on2)c1C] |
| 11 | 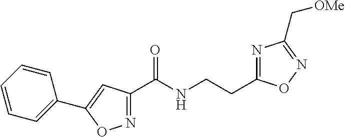 [SMILES: COCc1noc(CCNC(=O)c2cc(-c3ccccc3)on2)n1] |
| 12 | 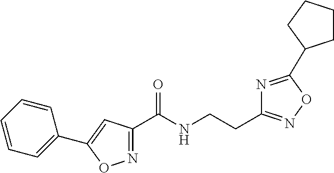 [SMILES: O=C(NCCc1noc(C2CCCC2)n1)c1cc(-c2ccccc2)on1] |
| 13 | 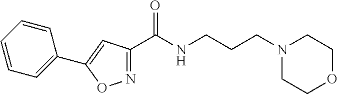 [SMILES: O=C(NCCCN1CCOCC1)c1cc(-c2ccccc2)on1] |
| 14 | 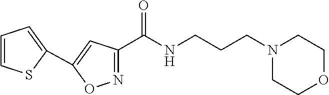 [SMILES: O=C(NCCCN1CCOCC1)c1cc(-c2cccs2)on1] |
| 15 | 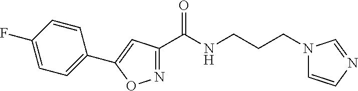 [SMILES: O=C(NCCCn1ccnc1)c1cc(-c2ccc(F)cc2)on1] |
| 16 | 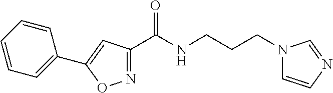 [SMILES: O=C(NCCCn1ccnc1)c1cc(-c2ccccc2)on1] |
| 17 | 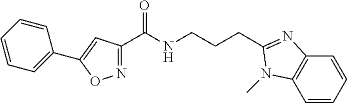 [SMILES: Cn1c(CCCNC(=O)c2cc(-c3ccccc3)on2)nc2ccccc21] |
| 18 | 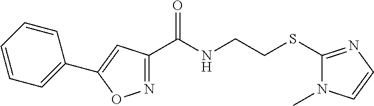 [SMILES: Cn1ccnc1SCCNC(=O)c1cc(-c2ccccc2)on1] |
| 19 | 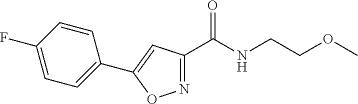 [SMILES: COCCNC(=O)c1cc(-c2ccc(F)cc2)on1] |

54. The method of claim 1, wherein the compound is selected from Compounds 20 to 371:
TABLE 2

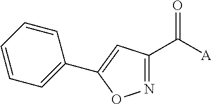

| Compound No. | A |
|---|---|
| 20 | 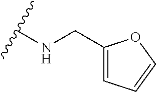 [SMILES: *NCc1ccco1] |
| 21 | 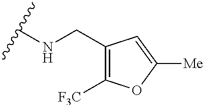 [SMILES: *NCc1cc(C)oc1C(F)(F)F] |
| 22 | 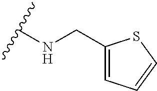 [SMILES: *NCc1cccs1] |
| 23 | 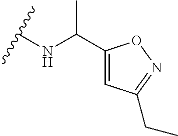 [SMILES: *NC(C)c1cc(CC)no1] |
| 24 | 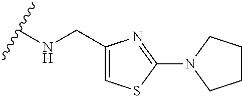 [SMILES: *NCc1csc(N2CCCC2)n1] |
| 25 | 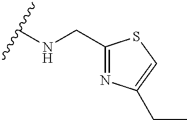 [SMILES: *NCc1nc(CC)cs1] |
| 26 | 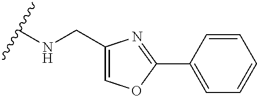 [SMILES: *NCc1coc(-c2ccccc2)n1] |
| 27 | 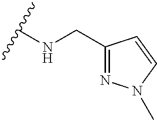 [SMILES: *NCc1ccn(C)n1] |
| 28 | 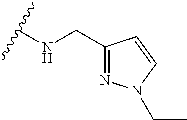 [SMILES: *NCc1ccn(CC)n1] |
| 29 | 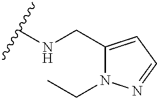 [SMILES: *NCc1ccnn1CC] |
| 30 | 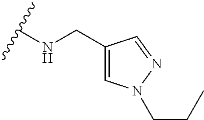 [SMILES: *NCc1cnn(CCC)c1] |
| 31 | 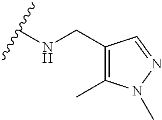 [SMILES: *NCc1cnn(C)c1C] |
| 32 | 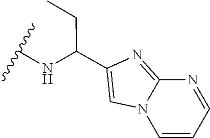 [SMILES: *NC(CC)c1cn2cccnc2n1] |
| 33 | 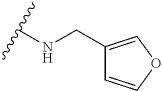 [SMILES: *NCc1ccoc1] |
| 34 | 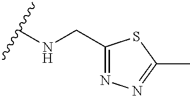 [SMILES: *NCc1nnc(C)s1] |
| 35 | 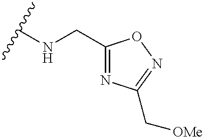 [SMILES: *NCc1nc(COC)no1] |
| 36 | 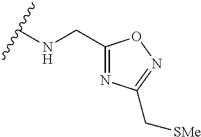 [SMILES: *NCc1nc(CSC)no1] |
| 37 | 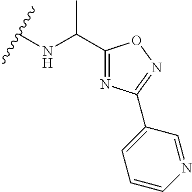 [SMILES: *NC(C)c1nc(-c2cccnc2)no1] |
| 38 | 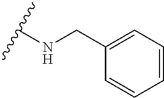 [SMILES: *NCc1ccccc1] |
| 39 | 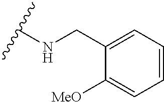 [SMILES: *NCc1ccccc1OC] |
| 40 | 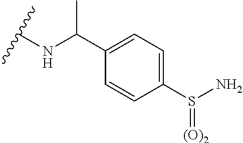 |
| 41 | 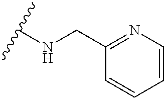 [SMILES: *NCc1ccccn1] |
| 42 | 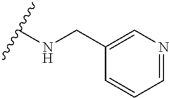 [SMILES: *NCc1cccnc1] |
| 43 | 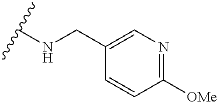 [SMILES: *NCc1ccc(OC)nc1] |
| 44 | 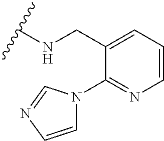 [SMILES: *NCc1cccnc1-n1ccnc1] |
| 45 | 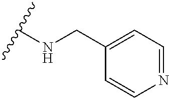 [SMILES: *NCc1ccncc1] |
| 46 | 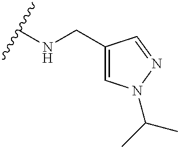 [SMILES: *NCc1cnn(C(C)C)c1] |
| 47 | 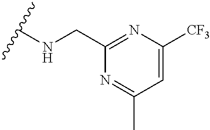 [SMILES: *NCc1nc(C)cc(C(F)(F)F)n1] |
| 48 | 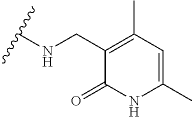 [SMILES: *NCc1c(C)cc(C)[nH]c1=O] |
| 49 | 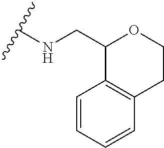 [SMILES: *NCC1OCCc2ccccc21] |
| 50 | 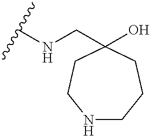 [SMILES: *NCC1(O)CCCNCC1] |
| 51 | 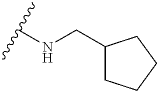 [SMILES: *NCC1CCCC1] |
| 52 | 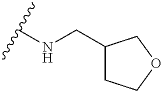 [SMILES: *NCC1CCOC1] |
| 53 | 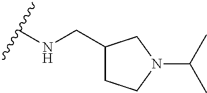 [SMILES: *NCC1CCN(C(C)C)C1] |
| 54 | 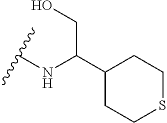 [SMILES: *NC(CO)C1CCSCC1] |
| 55 | 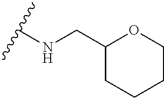 [SMILES: *NCC1CCCCO1] |
| 56 | 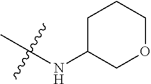 [SMILES: *NC1CCCOC1] |
| 57 | 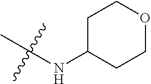 [SMILES: *NC1CCOCC1] |
| 58 | 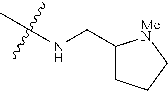 [SMILES: *NCC1CCCN1C] |
| 59 | 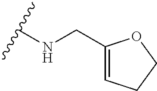 [SMILES: *NCC1=CCCO1] |
| 60 | 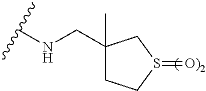 |

TABLE 3

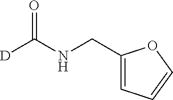

| Compound | |
|---|---|
| No. | D |
| 61 | 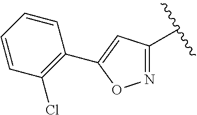 [SMILES: *c1cc(-c2ccccc2Cl)on1] |
| 62 | 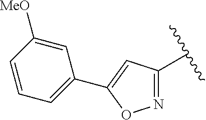 [SMILES: *c1cc(-c2cccc(OC)c2)on1] |
| 63 | 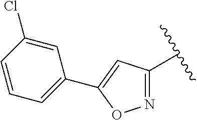 [SMILES: *c1cc(-c2cccc(Cl)c2)on1] |
| 64 | 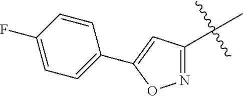 [SMILES: *c1cc(-c2ccc(F)cc2)on1] |
| 65 | 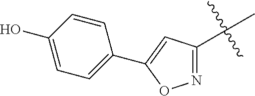 [SMILES: *c1cc(-c2ccc(O)cc2)on1] |
| 66 | 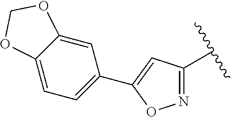 [SMILES: *c1cc(-c2ccc3c(c2)OCO3)on1] |
| 67 | 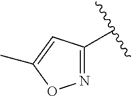 [SMILES: *c1cc(C)on1] |
| 68 | 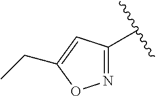 [SMILES: *c1cc(CC)on1] |
| 69 | 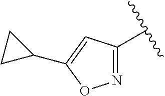 [SMILES: *c1cc(C2CC2)on1] |
| 70 | 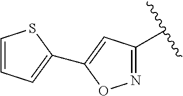 [SMILES: *c1cc(-c2cccs2)on1] |
| 71 | 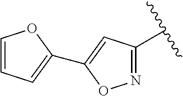 [SMILES: *c1cc(-c2ccco2)on1] |
| 72 | 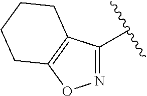 [SMILES: *c1noc2c1CCCC2] |

TABLE 4

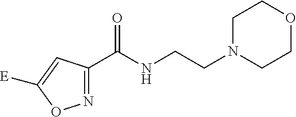

| Compound | |
|---|---|
| No. | E |
| 73 | 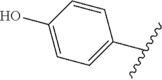 [SMILES: *c1ccc(O)cc1] |
| 74 | 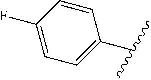 [SMILES: *c1ccc(F)cc1] |
| 75 | 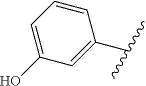 [SMILES: *c1cccc(O)c1] |
| 76 | 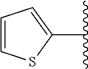 [SMILES: *c1cccs1] |
| 77 | 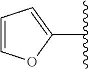 [SMILES: *c1ccco1] |

TABLE 5

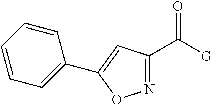

| Compound | |
|---|---|
| No. | G |
| 78 | 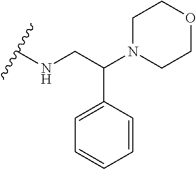 [SMILES: *NCC(c1ccccc1)N1CCOCC1] |
| 79 | 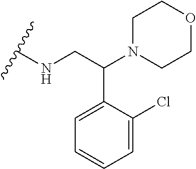 [SMILES: *NCC(c1ccccc1Cl)N1CCOCC1] |
| 80 | 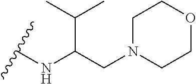 [SMILES: *NC(CN1CCOCC1)C(C)C] |
| 81 | 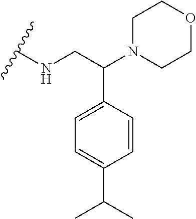 [SMILES: *NCC(c1ccc(C(C)C)cc1)N1CCOCC1] |
| 82 | 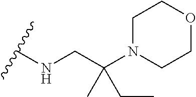 [SMILES: *NCC(C)(CC)N1CCOCC1] |

TABLE 6

| Compound | |
|---|---|
| No. | G′ |
| 83 | 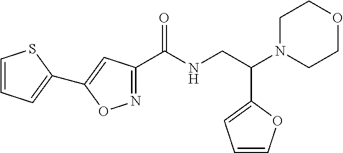 |
| 84 | 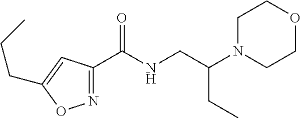 |
| 85 | 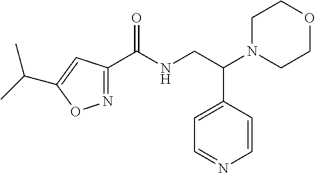 |
| 86 | 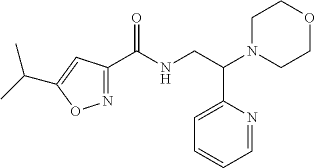 |

TABLE 7

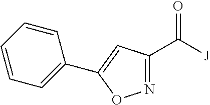

| Compound | |
|---|---|
| No. | J |
| 87 | 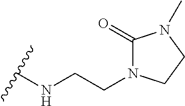 [SMILES: *NCCN1CCN(C)C1=O] |
| 88 | 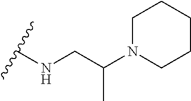 [SMILES: *NCC(C)N1CCCCC1] |
| 89 | 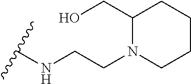 [SMILES: *NCCN1CCCCC1CO] |
| 90 | 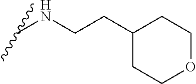 [SMILES: *NCCC1CCOCC1] |
| 91 | 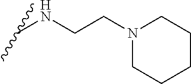 [SMILES: *NCCN1CCCCC1] |
| 92 | 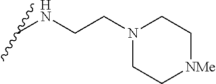 [SMILES: *NCCN1CCN(C)CC1] |
| 93 | 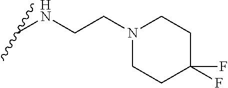 [SMILES: *NCCN1CCC(F)(F)CC1] |
| 94 | 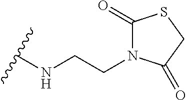 [SMILES: *NCCN1C(=O)CSC1=O] |
| 95 | 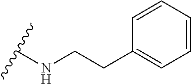 [SMILES: *NCCc1ccccc1] |
| 96 | 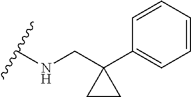 [SMILES: *NCC1(c2ccccc2)CC1] |
| 97 | 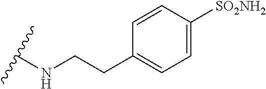 [SMILES: *NCCc1ccc(S(N)(=O)=O)cc1] |
| 98 | 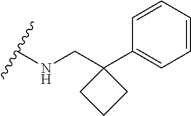 [SMILES: *NCC1(c2ccccc2)CCC1] |
| 99 | 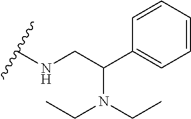 [SMILES: *NCC(c1ccccc1)N(CC)CC] |
| 100 | 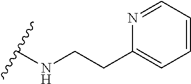 [SMILES: *NCCc1ccccn1] |
| 101 | 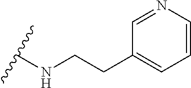 [SMILES: *NCCc1cccnc1] |
| 102 | 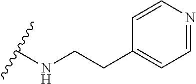 [SMILES: *NCCc1ccncc1] |
| 103 | 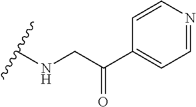 [SMILES: *NCC(=O)c1ccncc1] |
| 104 | 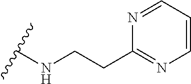 [SMILES: *NCCc1ncccn1] |
| 105 | 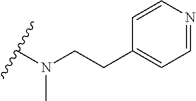 [SMILES: *N(C)CCc1ccncc1] |
| 106 | 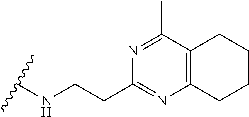 [SMILES: *NCCc1nc(C)c2c(n1)CCCC2] |
| 107 | 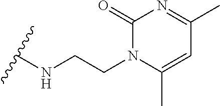 [SMILES: *NCCn1c(C)cc(C)nc1=O] |

TABLE 8

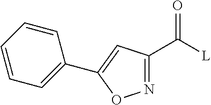

| Compound | |
|---|---|
| No. | L |
| 108 | 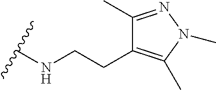 [SMILES: *NCCc1c(C)nn(C)c1C] |
| 109 | 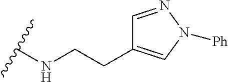 [SMILES: *NCCc1cnn(-c2ccccc2)c1] |
| 110 | 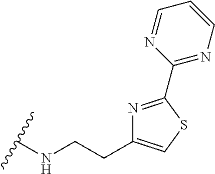 [SMILES: *NCCc1csc(-c2ncccn2)n1] |
| 111 | 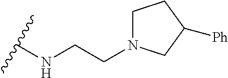 [SMILES: *NCCN1CCC(c2ccccc2)C1] |
| 112 | 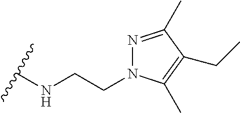 [SMILES: *NCCn1nc(C)c(CC)c1C] |
| 113 | 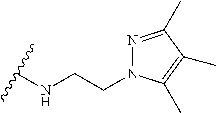 [SMILES: *NCCn1nc(C)c(C)c1C] |
| 114 | 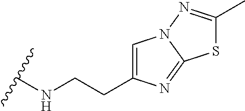 [SMILES: *NCCc1cn2nc(C)sc2n1] |
| 115 | 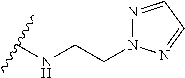 [SMILES: *NCCn1nccn1] |
| 116 | 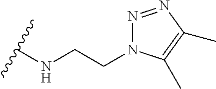 [SMILES: *NCCn1nnc(C)c1C] |
| 117 | 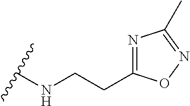 [SMILES: *NCCc1nc(C)no1] |
| 118 | 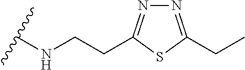 [SMILES: *NCCc1nnc(CC)s1] |
| 119 | 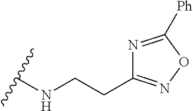 [SMILES: *NCCc1noc(-c2ccccc2)n1] |
| 120 | 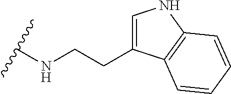 [SMILES: *NCCc1c[nH]c2ccccc12] |
| 121 | 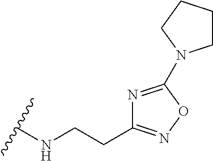 [SMILES: *NCCc1noc(N2CCCC2)n1] |
| 122 | 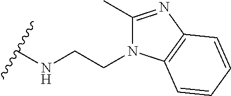 [SMILES: *NCCn1c(C)nc2ccccc21] |
| 123 | 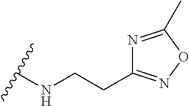 [SMILES: *NCCc1noc(C)n1] |

TABLE 9

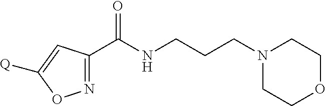

| Compound | |
|---|---|
| No. | Q |
| 124 | 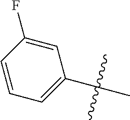 [SMILES: *c1cccc(F)c1] |
| 125 | 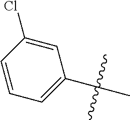 [SMILES: *c1cccc(Cl)c1] |
| 126 | 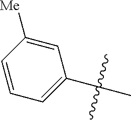 [SMILES: *c1cccc(C)c1] |
| 127 | 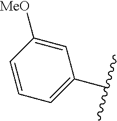 [SMILES: *c1cccc(OC)c1] |
| 128 | 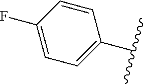 [SMILES: *c1ccc(F)cc1] |
| 129 | 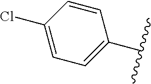 [SMILES: *c1ccc(Cl)cc1] |
| 130 | 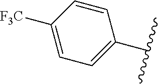 [SMILES: *c1ccc(C(F)(F)F)cc1] |
| 131 | 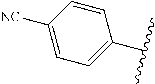 [SMILES: *c1ccc(C#N)cc1] |
| 132 | 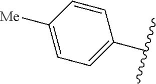 [SMILES: *c1ccc(C)cc1] |
| 133 | 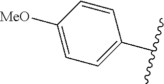 [SMILES: *c1ccc(OC)cc1] |
| 134 | 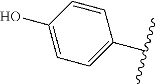 [SMILES: *c1ccc(O)cc1] |
| 135 | 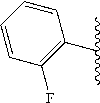 [SMILES: *c1ccccc1F] |
| 136 | 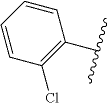 [SMILES: *c1ccccc1Cl] |
| 137 | 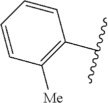 [SMILES: *c1ccccc1C] |
| 138 | 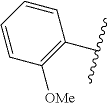 [SMILES: *c1ccccc1OC] |
| 139 | 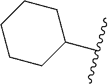 [SMILES: *C1CCCCC1] |
| 140 | 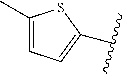 [SMILES: *c1ccc(C)s1] |
| 141 | 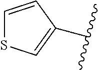 [SMILES: *c1ccsc1] |
| 142 | 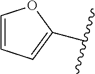 [SMILES: *c1ccco1] |

TABLE 10

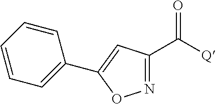

| Compound | |
|---|---|
| No. | Q′ |
| 143 | 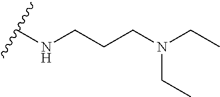 [SMILES: *NCCCN(CC)CC] |
| 144 | 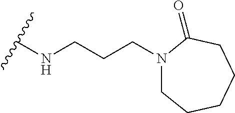 [SMILES: *NCCCN1CCCCCC1=O] |
| 145 | 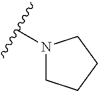 [SMILES: *N1CCCC1] |
| 146 | 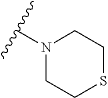 [SMILES: *N1CCSCC1] |
| 147 | 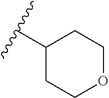 [SMILES: *C1CCOCC1] |
| 148 | 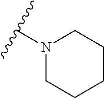 [SMILES: *N1CCCCC1] |
| 149 | 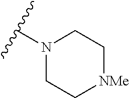 [SMILES: *N1CCN(C)CC1] |

TABLE 11

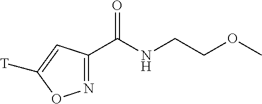

| Compound No. | T |
|---|---|
| 150 | 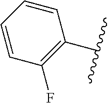 [SMILES: *c1ccccc1F] |
| 151 | 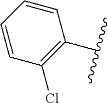 [SMILES: *c1ccccc1Cl] |
| 152 | 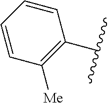 [SMILES: *c1ccccc1C] |
| 153 | 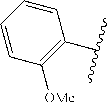 [SMILES: *c1ccccc1OC] |
| 154 | 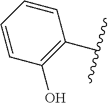 [SMILES: *c1ccccc1O] |
| 155 | 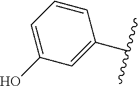 [SMILES: *c1cccc(O)c1] |
| 156 | 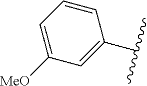 [SMILES: *c1cccc(OC)c1] |
| 157 | 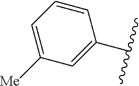 [SMILES: *c1cccc(C)c1] |
| 158 | 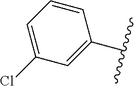 [SMILES: *c1cccc(Cl)c1] |
| 159 | 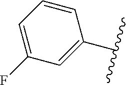 [SMILES: *c1cccc(F)c1] |
| 160 | 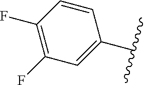 [SMILES: *c1ccc(F)c(F)c1] |
| 161 | 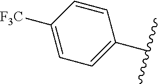 [SMILES: *c1ccc(C(F)(F)F)cc1] |
| 162 | 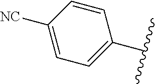 [SMILES: *c1ccc(C#N)cc1] |
| 163 | 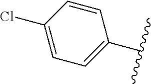 [SMILES: *c1ccc(Cl)cc1] |
| 164 | 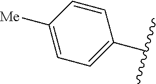 [SMILES: *c1ccc(C)cc1] |
| 165 | 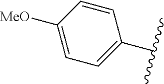 [SMILES: *c1ccc(OC)cc1] |
| 166 | 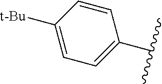 [SMILES: *c1ccc(C(C)(C)C)cc1] |
| 167 | 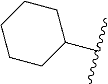 [SMILES: *C1CCCCC1] |
| 168 | 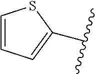 [SMILES: *c1cccs1] |
| 169 | 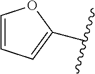 [SMILES: *c1ccco1] |
| 170 | 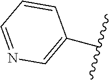 [SMILES: *c1cccnc1] |
| 171 | 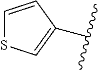 [SMILES: *c1ccsc1] |
| 329A | 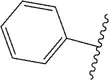 [SMILES: *c1ccccc1] |
| Compound 172 | 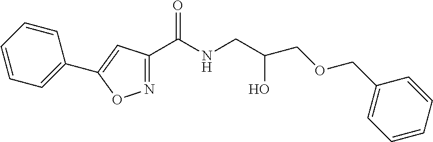 |
| Compound 173 | 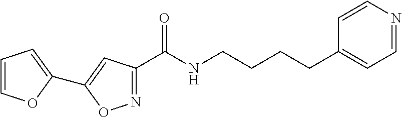 |

TABLE 12

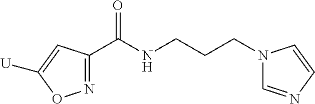

| Compound No. | U |
|---|---|
| 174 | 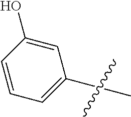 |
| 175 | 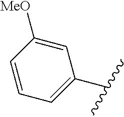 [SMILES: *c1cccc(OC)c1] |
| 176 | 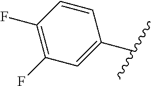 [SMILES: *c1ccc(F)c(F)c1] |
| 177 | 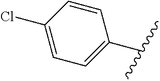 [SMILES: *c1ccc(Cl)cc1] |
| 178 | 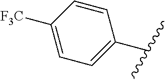 [SMILES: *c1ccc(C(F)(F)F)cc1] |
| 179 | 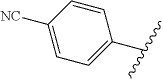 [SMILES: *c1ccc(C#N)cc1] |
| 180 | 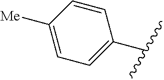 [SMILES: *c1ccc(C)cc1] |
| 181 | 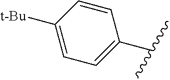 [SMILES: *c1ccc(C(C)(C)C)cc1] |
| 182 | 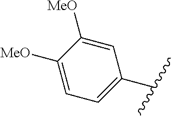 [SMILES: *c1ccc(OC)c(OC)c1] |
| 183 | 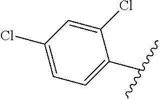 [SMILES: *c1ccc(Cl)cc1Cl] |
| 184 | 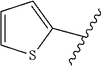 [SMILES: *c1cccs1] |
| 185 | 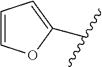 [SMILES: *c1ccco1] |

TABLE 13

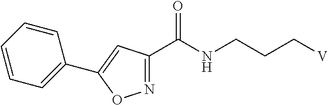

| Compound No. | V |
|---|---|
| 186 | 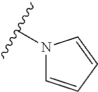 [SMILES: *n1cccc1] |
| 187 | 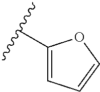 [SMILES: *c1ccco1] |
| 188 | 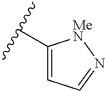 [SMILES: *c1ccnn1C] |
| 189 | 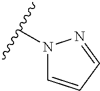 [SMILES: *n1cccn1] |
| 190 | 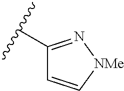 [SMILES: *c1ccn(C)n1] |
| 191 | 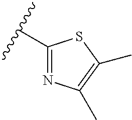 [SMILES: *c1nc(C)c(C)s1] |
| 192 | 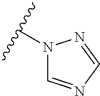 [SMILES: *n1cncn1] |
| 193 | 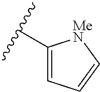 [SMILES: *c1cccn1C] |
| 194 | 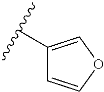 [SMILES: *c1ccoc1] |
| 195 | 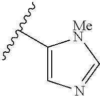 [SMILES: *c1cncn1C] |
| 196 | 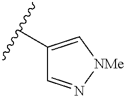 [SMILES: *c1cnn(C)c1] |
| 197 | 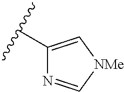 [SMILES: *c1cn(C)cn1] |
| 198 | 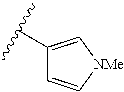 [SMILES: *c1ccn(C)c1] |
| 199 | 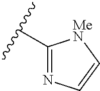 [SMILES: *c1nccn1C] |
| 200 | 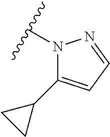 [SMILES: *n1nccc1C1CC1] |
| 201 | 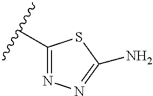 [SMILES: *c1nnc(N)s1] |
| 202 | 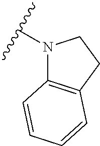 [SMILES: *N1CCc2ccccc21] |
| 203 | 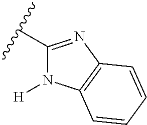 [SMILES: *c1nc2ccccc2[nH]1] |
| 204 | 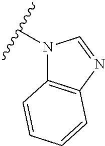 [SMILES: *n1cnc2ccccc21] |
| 205 | 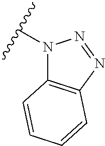 [SMILES: *n1nnc2ccccc21] |
| 206 | 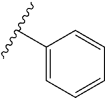 [SMILES: *c1ccccc1] |
| 207 | 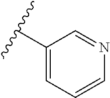 [SMILES: *c1cccnc1] |
| 208 | 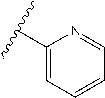 [SMILES: *c1ccccn1] |
| 209 | 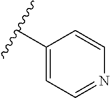 [SMILES: *c1ccncc1] |
| 210 | 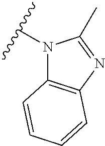 [SMILES: *n1c(C)nc2ccccc21] |
| 211 | 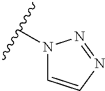 [SMILES: *n1ccnn1] |

TABLE 14

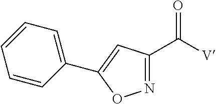

| Compound No. | V′ |
|---|---|
| 212 | 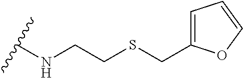 [SMILES: *NCCSCc1ccco1] |
| 213 | 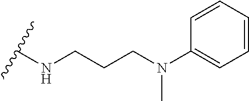 [SMILES: *NCCCN(C)c1ccccc1] |
| 214 | 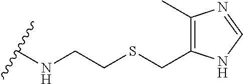 [SMILES: *NCCSCc1[nH]cnc1C] |
| 215 | 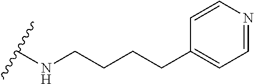 [SMILES: *NCCCCc1ccncc1] |
| 216 | 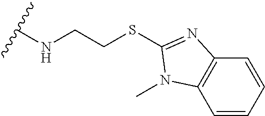 [SMILES: *NCCSc1nc2ccccc2n1C] |
| 217 | 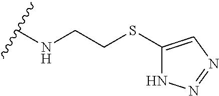 [SMILES: *NCCSc1cnn[nH]1] |
| 218 | 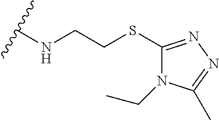 [SMILES: *NCCSc1nnc(C)n1CC] |
| 219 | 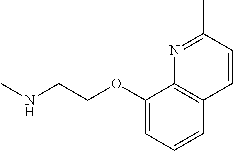 |
| 220 | 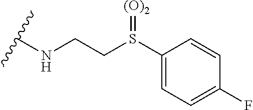 |
| 221 | 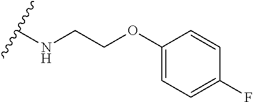 [SMILES: *NCCOc1ccc(F)cc1] |
| 222 | 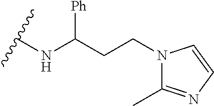 [SMILES: *NC(CCn1ccnc1C)c1ccccc1] |

TABLE 15

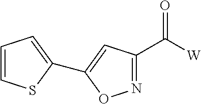

| Compound No. | W |
|---|---|
| 223 | 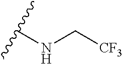 [SMILES: *NCC(F)(F)F] |
| 224 | 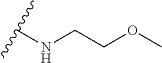 [SMILES: *NCCOC] |
| 225 | 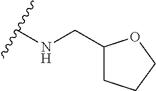 [SMILES: *NCC1CCCO1] |
| 226 | 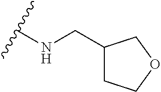 [SMILES: *NCC1CCOC1] |
| 227 | 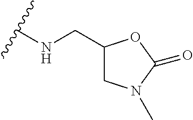 [SMILES: *NCC1CN(C)C(=O)O1] |
| 228 | 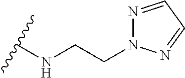 [SMILES: *NCCn1nccn1] |
| 229 | 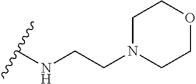 [SMILES: *NCCN1CCOCC1] |
| 230 | 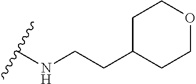 [SMILES: *NCCC1CCOCC1] |
| 231 | 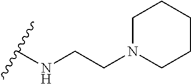 [SMILES: *NCCN1CCCCC1] |
| 232 | 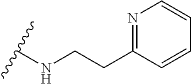 [SMILES: *NCCc1ccccn1] |
| 233 | 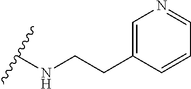 [SMILES: *NCCc1cccnc1] |
| 234 | 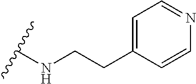 [SMILES: *NCCc1ccncc1] |
| 235 | 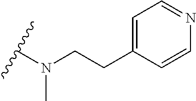 [SMILES: *N(C)CCc1ccncc1] |
| 236 | 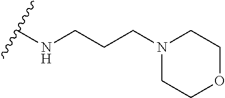 [SMILES: *NCCCN1CCOCC1] |
| 237 | 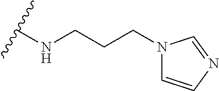 [SMILES: *NCCCn1ccnc1] |
| 238 | 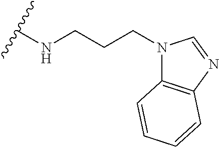 [SMILES: *NCCCn1cnc2ccccc21] |
| 239 | 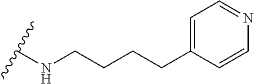 [SMILES: *NCCCCc1ccncc1] |
| 240 | 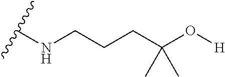 [SMILES: *NCCCC(C)(C)O] |

TABLE 16

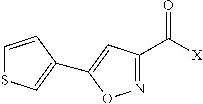

| Compound No. | X |
|---|---|
| 241 | 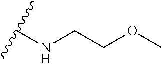 [SMILES: *NCCOC] |
| 242 | 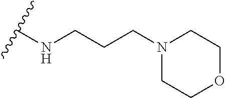 [SMILES: *NCCCN1CCOCC1] |

TABLE 17

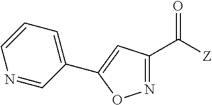

| Compound No. | Z |
|---|---|
| 243 | 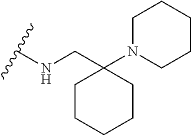 [SMILES: *NCC1(N2CCCCC2)CCCCC1] |
| 244 | 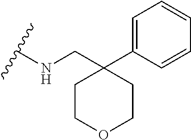 [SMILES: *NCC1(c2ccccc2)CCOCC1] |
| 245 | 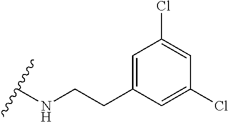 [SMILES: *NCCc1cc(Cl)cc(Cl)c1] |
| 246 | 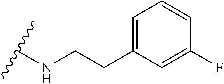 [SMILES: *NCCc1cccc(F)c1] |
| 247 | 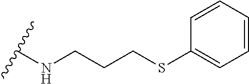 [SMILES: *NCCCSc1ccccc1] |
| 248 | 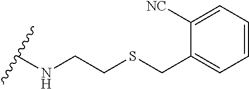 [SMILES: *NCCSCc1ccccc1C#N] |

TABLE 18

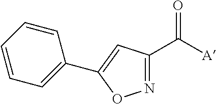

| Compond No. | A′ |
|---|---|
| 249 | 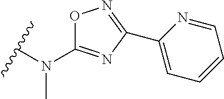 [SMILES: *N(C)c1nc(-c2ccccn2)no1] |
| 250 | 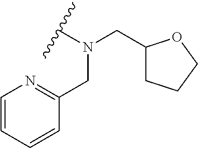 [SMILES: *N(Cc1ccccn1)CC1CCCO1] |
| 251 | 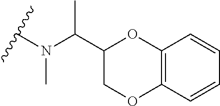 [SMILES: *N(C)C(C)C1COc2ccccc2O1] |
| 252 | 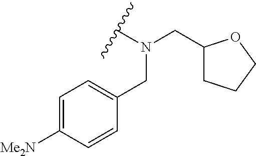 [SMILES: *N(Cc1ccc(N(C)C)cc1)CC1CCCO1] |
| 253 | 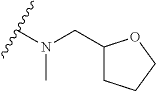 [SMILES: *N(C)CC1CCCO1] |
| 254 | 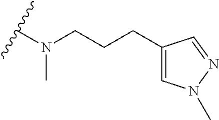 [SMILES: *N(C)CCCc1cnn(C)c1] |
| Compound 255 | 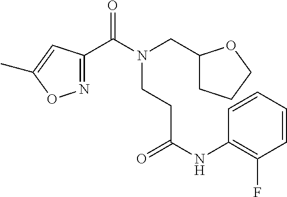 |
| Compound 256 | 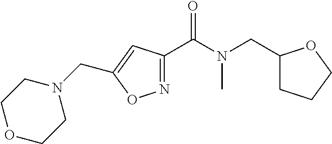 |
| Compound 257 | 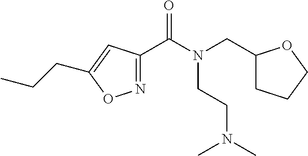 |

TABLE 19

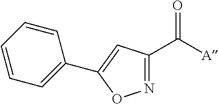

| Compound No. | A″ |
|---|---|
| 258 | 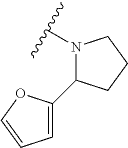 [SMILES: *N1CCCC1c1ccco1] |
| 259 | 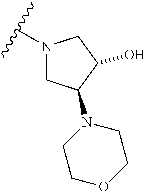 [SMILES: *N1C[C@H](O)[C@@H](N2CCOCC2)C1] |
| 260 | 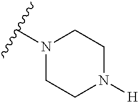 [SMILES: *N1CCNCC1] |
| 261 | 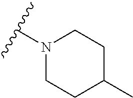 [SMILES: *N1CCC(C)CC1] |
| 262 | 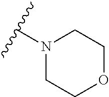 [SMILES: *N1CCOCC1] |
| 263 | 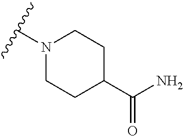 [SMILES: *N1CCC(C(N)=O)CC1] |
| 264 | 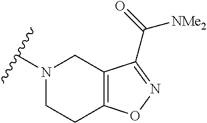 [SMILES: *N1CCc2onc(C(=O)N(C)C)c2C1] |
| 265 | 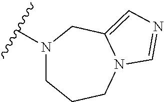 [SMILES: *N1CCCn2cncc2C1] |
| 266 | 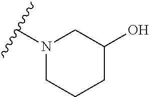 [SMILES: *N1CCCC(O)C1] |
| 267 | 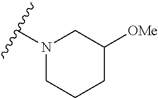 [SMILES: *N1CCCC(OC)C1] |
| Compound 268 | 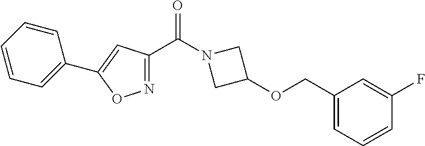 |
| Compound 269 | 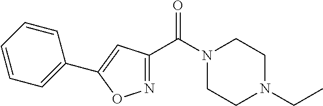 |

TABLE 20

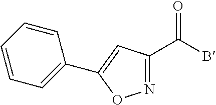

| Compound No. | B′ |
|---|---|
| 270 | 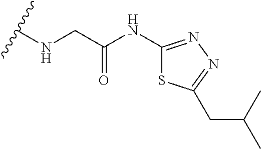 [SMILES: *NCC(=O)Nc1nnc(CC(C)C)s1] |
| 271 | 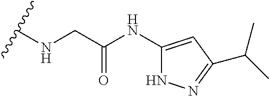 [SMILES: *NCC(=O)Nc1cc(C(C)C)n[nH]1] |
| 272 | 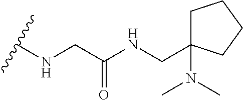 [SMILES: *NCC(=O)NCC1(N(C)C)CCCC1] |
| 273 | 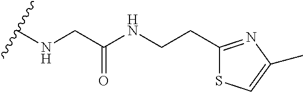 [SMILES: *NCC(=O)NCCc1nc(C)cs1] |
| 274 | 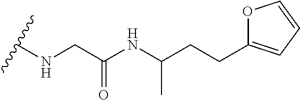 [SMILES: *NCC(=O)NC(C)CCc1ccco1] |
| 275 | 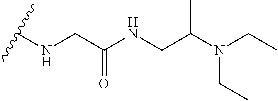 [SMILES: *NCC(=O)NCC(C)N(CC)CC] |
| 276 | 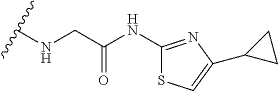 [SMILES: *NCC(=O)Nc1nc(C2CC2)cs1] |
| 277 | 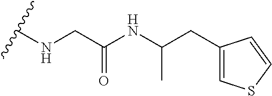 [SMILES: *NCC(=O)NC(C)Cc1ccsc1] |
| 278 | 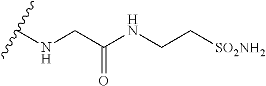 [SMILES: *NCC(=O)NCCS(N)(=O)=O] |
| 279 | 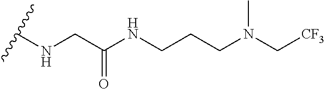 [SMILES: *NCC(=O)NCCCN(C)CC(F)(F)F] |
| 280 | 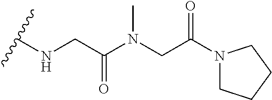 [SMILES: *NCC(=O)N(C)CC(=O)N1CCCC1] |
| 281 | 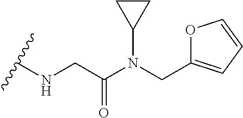 [SMILES: *NCC(=O)N(Cc1ccco1)C1CC1] |
| 282 | 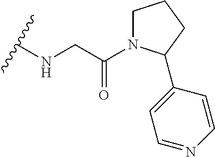 [SMILES: *NCC(=O)N1CCCC1c1ccncc1] |
| 283 | 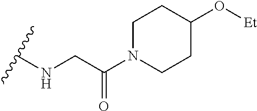 [SMILES: *NCC(=O)N1CCC(OCC)CC1] |
| 284 | 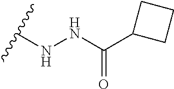 [SMILES: *NNC(=O)C1CCC1] |
| 285 | 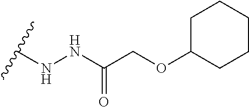 [SMILES: *NNC(=O)COC1CCCCC1] |
| 286 | 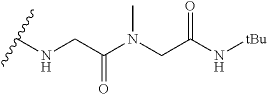 [SMILES: *NCC(=O)N(C)CC(=O)NC(C)(C)C] |
| 287 | 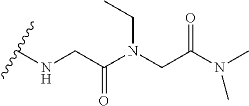 [SMILES: *NCC(=O)N(CC)CC(=O)N(C)C] |
| 288 | 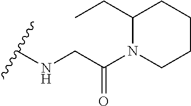 [SMILES: *NCC(=O)N1CCCCC1CC] |
| 289 | 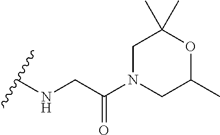 [SMILES: *NCC(=O)N1CC(C)OC(C)(C)C1] |
| 290 | 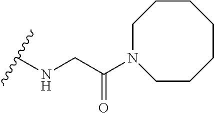 [SMILES: *NCC(=O)N1CCCCCCC1] |
| 291 | 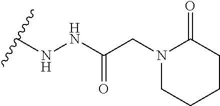 [SMILES: *NNC(=O)CN1CCCCC1=O] |
| 292 | 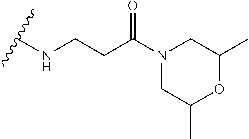 [SMILES: *NCCC(=O)N1CC(C)OC(C)C1] |

TABLE 21

| Com- | |
|---|---|
| pound | |
| No. | |
| 293 | 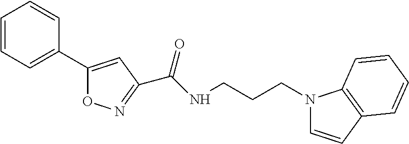 [SMILES: O=C(NCCCn1ccc2ccccc21)c1cc(-c2ccccc2)on1] |
| 294 | 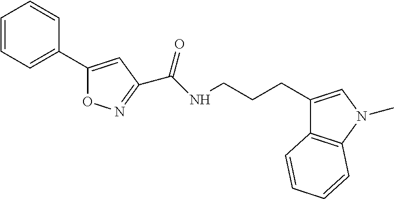 [SMILES: Cn1cc(CCCNC(=O)c2cc(-c3ccccc3)on2)c2ccccc21] |
| 295 | 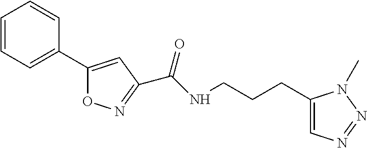 [SMILES: Cn1nncc1CCCNC(=O)c1cc(-c2ccccc2)on1] |
| 296 | 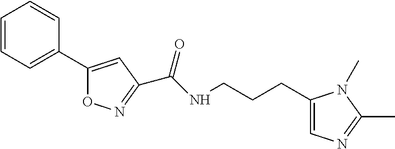 [SMILES: Cc1ncc(CCCNC(=O)c2cc(-c3ccccc3)on2)n1C] |
| 297 | 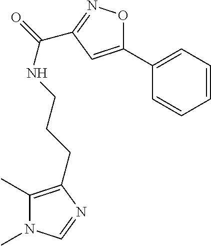 [SMILES: Cc1c(CCCNC(=O)c2cc(-c3ccccc3)on2)ncn1C] |
| 298 | 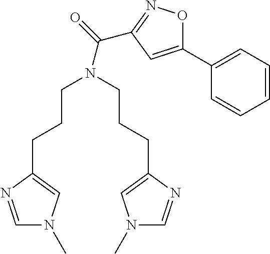 [SMILES: Cn1cnc(CCCN(CCCc2cn(C)cn2)C(=O)c2cc(-c3ccccc3)on2)c1] |
| 299 | 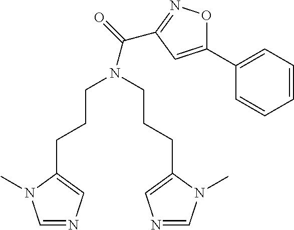 [SMILES: Cn1cncc1CCCN(CCCc1cncn1C)C(=O)c1cc(-c2ccccc2)on1] |
| 300 | 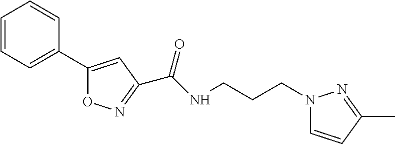 [SMILES: Cc1ccn(CCCNC(=O)c2cc(-c3ccccc3)on2)n1] |
| 301 | 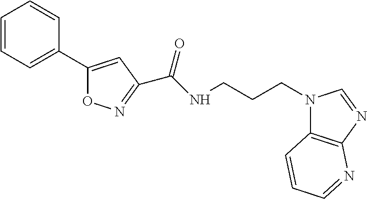 [SMILES: O=C(NCCCn1cnc2ncccc21)c1cc(-c2ccccc2)on1] |
| 302 | 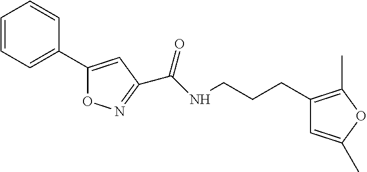 [SMILES: Cc1cc(CCCNC(=O)c2cc(-c3ccccc3)on2)c(C)o1] |
| 303 | 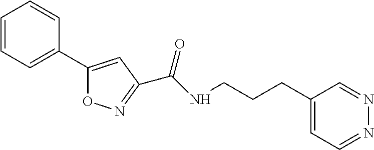 [SMILES: O=C(NCCCc1ccnnc1)c1cc(-c2ccccc2)on1] |

TABLE 22

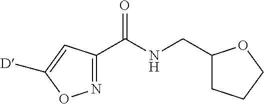

| Compound No. | D′ |
|---|---|
| 304 | 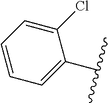 [SMILES: *c1ccccc1Cl] |
| 305 | 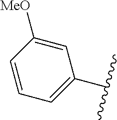 [SMILES: *c1cccc(OC)c1] |
| 306 | 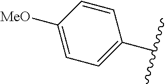 [SMILES: *c1ccc(OC)cc1] |
| 307 | 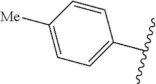 [SMILES: *c1ccc(C)cc1] |
| 308 | 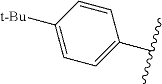 [SMILES: *c1ccc(C(C)(C)C)cc1] |
| 309 | 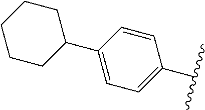 [SMILES: *c1ccc(C2CCCCC2)cc1] |
| 310 | 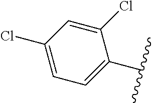 [SMILES: *c1ccc(Cl)cc1Cl] |
| 311 | H |
| 312 | Me |
| 313 | 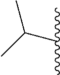 [SMILES: *C(C)C] |
| 314 | 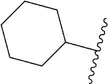 [SMILES: *C1CCCCC1] |
| 315 | 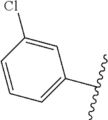 [SMILES: *c1cccc(Cl)c1] |
| 316 | 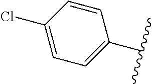 [SMILES: *c1ccc(Cl)cc1] |
| 317 | 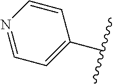 [SMILES: *c1ccncc1] |
| 318 | 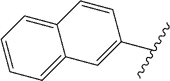 [SMILES: *c1ccc2ccccc2c1] |
| 319 | 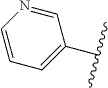 [SMILES: *c1cccnc1] |

TABLE 23

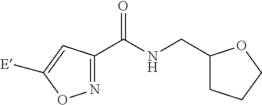

| Compound No. | E′ |
|---|---|
| 320 | 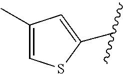 [SMILES: *c1cc(C)cs1] |
| 321 | 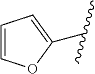 [SMILES: *c1ccco1] |
| 322 | 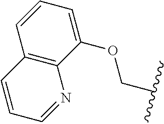 [SMILES: *COc1cccc2cccnc12] |
| 323 | 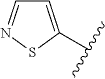 [SMILES: *c1ccns1] |
| 324 | 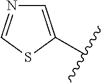 [SMILES: *c1cncs1] |
| 325 | 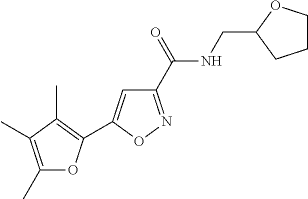 |
| 326 | 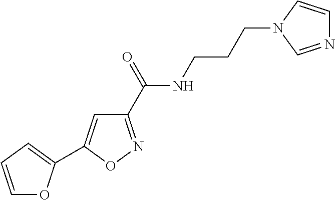 |
| 327 | 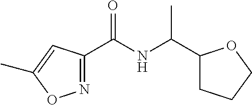 |
| 328 | 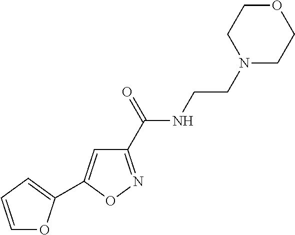 |

TABLE 24

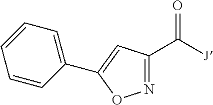

| Compound No. | J′ |
|---|---|
| 329B | 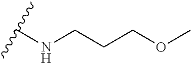 [SMILES: *NCCCOC] |
| 330 | 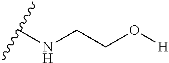 [SMILES: *NCCO] |
| 331 | 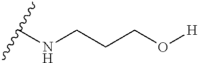 [SMILES: *NCCCO] |
| 332 | 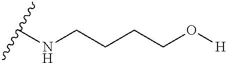 [SMILES: *NCCCCO] |
| 333 | 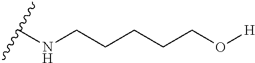 [SMILES: *NCCCCCO] |
| 334 | 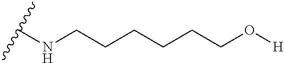 [SMILES: *NCCCCCCO] |
| 335 | 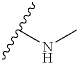 [SMILES: *NC] |
| 336 | 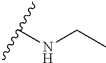 [SMILES: *NCC] |
| 337 | 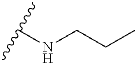 [SMILES: *NCCC] |
| 338 | 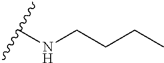 [SMILES: *NCCCC] |
| 339 | 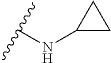 [SMILES: *NC1CC1] |
| 340 | 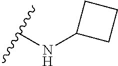 [SMILES: *NC1CCC1] |
| 341 | 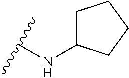 [SMILES: *NC1CCCC1] |
| Compound 342 | 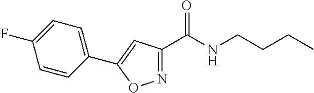 |
| Compound 343 | 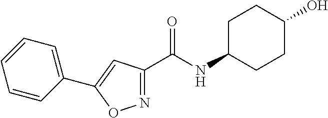 |
| Compound 344 | 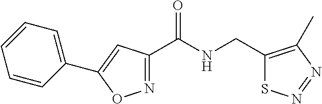 |
| Compound 345 | 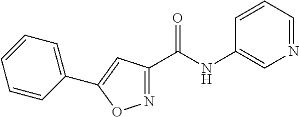 |

TABLE 25

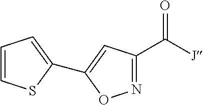

| Compound No. | J″ |
|---|---|
| 346 | 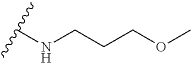 [SMILES: *NCCCOC] |
| 347 | 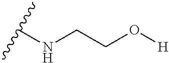 [SMILES: *NCCO] |
| 348 | 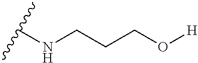 [SMILES: *NCCCO] |
| 349 | 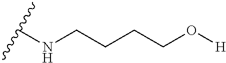 [SMILES: *NCCCCO] |
| 350 | 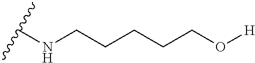 [SMILES: *NCCCCCO] |
| 351 | 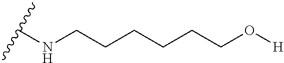 [SMILES: *NCCCCCCO] |
| 352 | 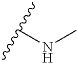 [SMILES: *NC] |
| 353 | 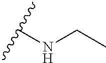 [SMILES: *NCC] |
| 354 | 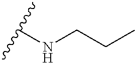 [SMILES: *NCCC] |
| 355 | 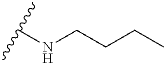 [SMILES: *NCCCC] |
| 356 | 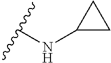 [SMILES: *NC1CC1] |
| 357 | 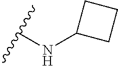 [SMILES: *NC1CCC1] |
| 358 | 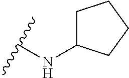 [SMILES: *NC1CCCC1] |

TABLE 26

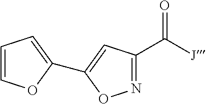

| Compound No. | J′″ |
|---|---|
| 359 | 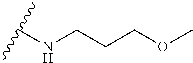 [SMILES: *NCCCOC] |
| 360 | 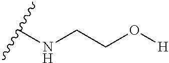 [SMILES: *NCCO] |
| 361 | 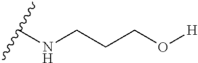 [SMILES: *NCCCO] |
| 362 | 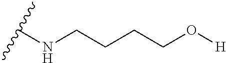 [SMILES: *NCCCCO] |
| 363 | 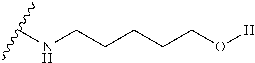 [SMILES: *NCCCCCO] |
| 364 | 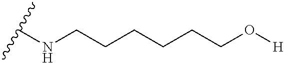 [SMILES: *NCCCCCCO] |
| 365 | 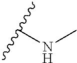 [SMILES: *NC] |
| 366 | 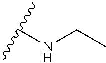 [SMILES: *NCC] |
| 367 | 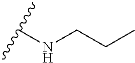 [SMILES: *NCCC] |
| 368 | 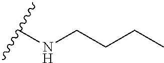 [SMILES: *NCCCC] |
| 369 | 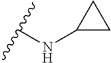 [SMILES: *NC1CC1] |
| 370 | 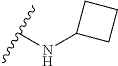 [SMILES: *NC1CCC1] |
| 371 | 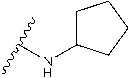 [SMILES: *NC1CCCC1] |
| Compound 372 | 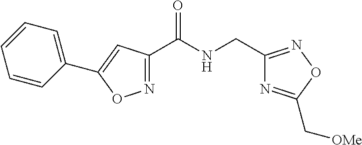 |
| Compound 373 | 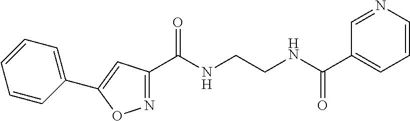 |
| Compound 374 | 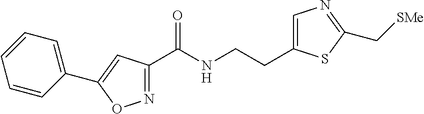 |
| Compound 375 | 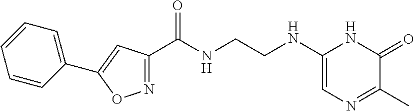 |
| Compound 376 | 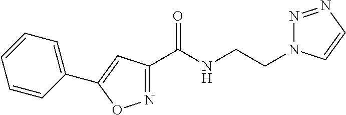 |
| Compound 377 | 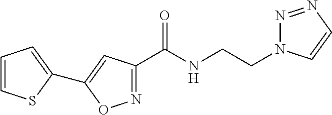 |
| Compound 378 | 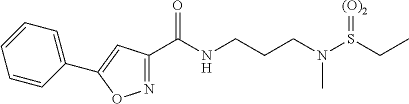 |

55. The method of claim 1, wherein the CFTR activity is enhanced.

56. The method of claim 1, wherein the activity of a mutant CFTR is enhanced.

57. The method of claim 1, wherein ΔF508 CFTR activity is modulated.

58. The method of claim 55, wherein ΔF508 CFTR activity is enhanced.

59. The method of claim 1, wherein the subject is suffering from a disease associated with decreased CFTR activity.

60. The method of claim 59, wherein the disease is cystic fibrosis.

61. The method of claim 59, wherein the subject is a human patient.

62. The method of claim 1, wherein the CFTR activity is suppressed.

63. The method of claim 62, wherein the subject is suffering from a disease that can be ameliorated by suppressing CFTR activity.

64. The method of claim 55, further comprising administering an additional therapeutic agent.

65. The method of claim 64, wherein at least two additional therapeutic agents are administered.

66. The method of claim 64, wherein the CFTR activity is enhanced and at least one additional therapeutic agent is a CFTR corrector or potentiator.

67. The method of claim 66, wherein each CFTR corrector or potentiator is independently selected from the group consisting of VX-770 (Ivacaftor), VX-809 (3-(6-(1-(2,2-difluorobenzo[d][1,3]dioxol-5-yl)cyclopropanecarboxamido)-3-methylpyridin-2-yl)benzoic acid) and VX-983.

68. An enantiomerically pure compound selected from (S)-5-phenyl-N-((tetrahydrofuran-2-yl)methyl)isoxazole-3-carboxamide and (R)-5-phenyl-N-((tetrahydrofuran-2-yl)methyl)isoxazole-3-carboxamide:

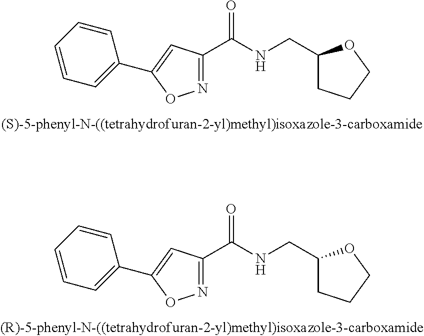 [SMILES: O=C(NC[C@@H]1CCCO1)c1cc(-c2ccccc2)on1 ; O=C(NC[C@H]1CCCO1)c1cc(-c2ccccc2)on1]

69. The compound of claim 68, wherein the compound is (S)-5-phenyl-N-((tetrahydrofuran-2-yl)methyl)isoxazole-3-carboxamide.

70. The compound of claim 68, wherein the compound is (R)-5-phenyl-N-((tetrahydrofuran-2-yl)methyl)isoxazole-3-carboxamide.

71. A compound selected from those shown in the Table below:
TABLE 1A

| Com- | |
|---|---|
| pound | |
| No | |
| 20 | 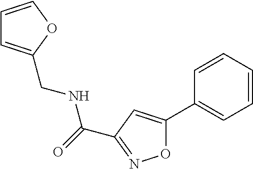 [SMILES: O=C(NCc1ccco1)c1cc(-c2ccccc2)on1] |
| 90 | 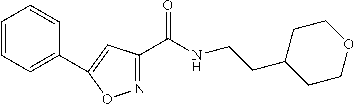 [SMILES: O=C(NCCC1CCOCC1)c1cc(-c2ccccc2)on1] |
| 92 | 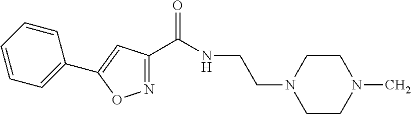 [SMILES: CN1CCN(CCNC(=O)c2cc(-c3ccccc3)on2)CC1] |
| 115 | 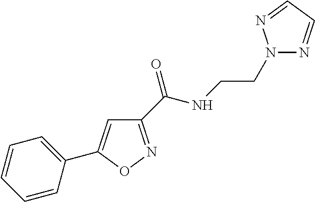 [SMILES: O=C(NCCn1nccn1)c1cc(-c2ccccc2)on1] |
| 135 | 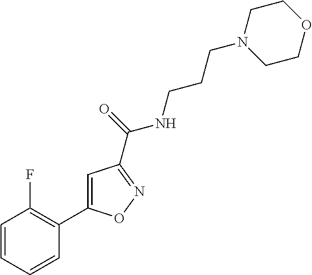 [SMILES: O=C(NCCCN1CCOCC1)c1cc(-c2ccccc2F)on1] |
| 188 | 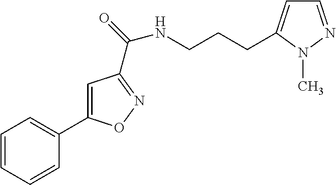 [SMILES: Cn1nccc1CCCNC(=O)c1cc(-c2ccccc2)on1] |
| 194 | 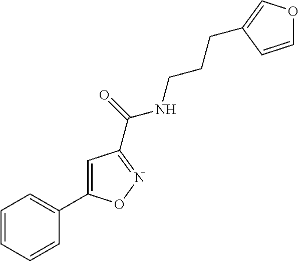 [SMILES: O=C(NCCCc1ccoc1)c1cc(-c2ccccc2)on1] |
| 195 | 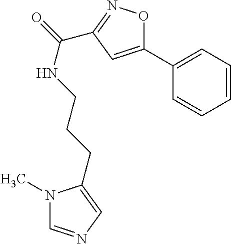 [SMILES: Cn1cncc1CCCNC(=O)c1cc(-c2ccccc2)on1] |
| 197 | 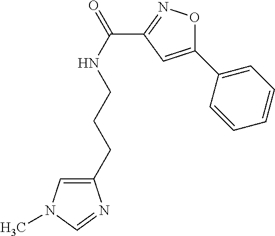 [SMILES: Cn1cnc(CCCNC(=O)c2cc(-c3ccccc3)on2)c1] |
| 198 | 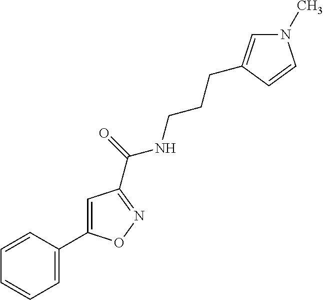 [SMILES: Cn1ccc(CCCNC(=O)c2cc(-c3ccccc3)on2)c1] |
| 226 | 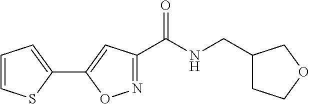 [SMILES: O=C(NCC1CCOC1)c1cc(-c2cccs2)on1] |
| 230 | 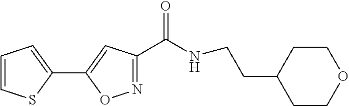 [SMILES: O=C(NCCC1CCOCC1)c1cc(-c2cccs2)on1] |
| 336 | 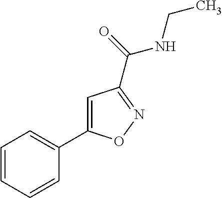 [SMILES: CCNC(=O)c1cc(-c2ccccc2)on1] |
| 349 | 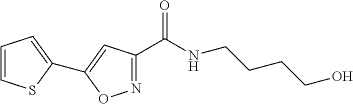 [SMILES: O=C(NCCCCO)c1cc(-c2cccs2)on1] |
| 376 | 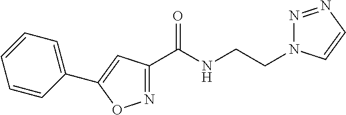 [SMILES: O=C(NCCn1ccnn1)c1cc(-c2ccccc2)on1] |

72. A pharmaceutical composition comprising a compound of claim 1, and a pharmaceutically acceptable carrier.
